(19) United States
(12) Patent Application Publication
(10) Pub. No.: US 2024/0180019 A1
(43) Pub. Date: May 30, 2024

(54) OPTO-ELECTRONIC DEVICE AND ELECTRONIC APPARATUS INCLUDING THE SAME

(71) Applicant: Samsung Display Co., Ltd., Yongin-Si (KR)

(72) Inventors: Seran Kim, Yongin-si (KR); Minsoo Choi, Yongin-si (KR); Hoilim Kim, Yongin-si (KR); Dongsun Yoo, Yongin-si (KR); Seokgyu Yoon, Yongin-si (KR); Jinsoo Jung, Yongin-si (KR); Hyejin Jung, Yongin-si (KR); Youngeun Choi, Yongin-si (KR)

(21) Appl. No.: 18/208,310

(22) Filed: Jun. 12, 2023

(30) Foreign Application Priority Data
Nov. 7, 2022 (KR) 10-2022-0147371

(51) Int. Cl. H10K 85/30; C07D 333/06; C07D 471/06; C07D 493/06; C07D 517/16; C07F 5/02; C09K 11/06; H10K 30/20; H10K 85/60

(52) U.S. Cl. CPC H10K 85/322; C07D 333/06; C07D 471/06; C07D 493/06; C07D 517/16; C07F 5/022; C09K 11/06; H10K 30/20; H10K 85/621; H10K 85/654; H10K 85/655; H10K 85/657; H10K 85/6572; H10K 85/6574; H10K 2101/40

(57) ABSTRACT

Provided is an opto-electronic device including a first electrode, a second electrode facing the first electrode, and a photoactive layer arranged between the first electrode and the second electrode, wherein the photoactive layer includes a first layer and a second layer, the first layer including a first compound, and the second layer including a second compound that does not include a 5-membered ring, the first compound is an electron-donating compound, the second compound is an electron-accepting compound, and the opto-electronic device satisfies Equation 1:

0.1≤(E¹_{LUMO}−E²_{LUMO})/(E¹_{LUMO}−E¹_{HOMO})≤0.5 Equation 1
wherein, in Equation 1, E¹_{LUMO}, E¹_{HOMO}, and E²_{LUMO} are each as defined herein.

### CROSS-REFERENCE TO RELATED APPLICATION

This application is based on and claims priority under 35 U.S.C. § 119 to Korean Patent Application No. 10-2022-0147371 filed on Nov. 7, 2022 in the Korean Intellectual Property Office, the disclosure of which is incorporated by reference herein in its entirety.

### BACKGROUND

### 1. Field

One or more embodiments relate to an opto-electronic device and an electronic apparatus including the same.

### 2. Description of the Related Art

Opto-electronic devices are devices that convert light energy or a light signal into electrical energy or an electrical signal. Examples of opto-electronic devices include photovoltaic cells or solar cells that convert light energy into electrical energy, photodetectors or light sensors that detect light energy and convert the detected light energy into an electrical signal, and the like.

Electronic apparatuses including opto-electronic devices and light-emitting devices have been developed. For example, light emitted from a light-emitting device may be reflected by an object (e.g., a finger of a user) in contact with an electronic apparatus, and then incident on an opto-electronic device. As the opto-electronic device detects incident light energy and converts the detected incident light energy into an electrical signal, it may be recognized that the object is in contact with the electronic apparatus. The opto-electronic device may be used as a fingerprint recognition sensor or the like.

The external quantum efficiency (EQE) of an opto-electronic device may be a measure of the ratio of current generated to light absorbed. The dark current density (J_{dark}) of an opto-electronic device represents current generated by heat or the like, rather than light, and thus may be a measure of noise in the opto-electronic device. There is a demand for opto-electronic devices having improved photoelectric characteristics such as external quantum efficiency and dark current density.

### SUMMARY

One or more embodiments include an opto-electronic device having improved photoelectric characteristics and a high-quality electronic apparatus including the opto-electronic device.

Additional aspects will be set forth in part in the description which follows and, in part, will be apparent from the description, or may be learned by practice of the presented embodiments of the disclosure.

According to one or more embodiments, an opto-electronic device includes a first electrode, a second electrode facing the first electrode, and a photoactive layer arranged between the first electrode and the second electrode, wherein the photoactive layer includes a first layer and a second layer, the first layer including a first compound, and the second layer including a second compound that does not include a 5-membered ring, the first compound is an electron-donating compound, the second compound is an electron-accepting compound, and the opto-electronic device satisfies Equation 1:

0.1≤(E¹_{LUMO}−E²_{LUMO})/(E¹_{LUMO}−E¹_{HOMO})≤0.5 Equation 1

wherein, in Equation 1,
E¹_{LUMO} indicates a lowest unoccupied molecular orbital (LUMO) energy level of the first compound,
E¹_{HOMO} indicates a highest occupied molecular orbital (HOMO) energy level of the first compound, and
E²_{LUMO} indicates a LUMO energy level of the second compound.

According to one or more embodiments, an electronic apparatus includes the opto-electronic device.

According to one or more embodiments, an electronic device includes the electronic apparatus.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of an opto-electronic device according to an embodiment;

FIG. 2 is a schematic view of a light-emitting device included in an electronic apparatus according to an embodiment;

FIG. 3 is a schematic view of an electronic apparatus according to an embodiment;

FIG. 4 is a schematic view of an electronic apparatus according to another embodiment;

FIG. 5 is a schematic perspective view of an electronic device according to an embodiment;

FIG. 6 is a schematic view of the exterior of a vehicle as an electronic device according to another embodiment;

FIGS. 7A to 7C are schematic views of the interior of the vehicle of FIG. 6; and

FIG. 8 is a cross-sectional view of an electronic apparatus according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

An aspect of the disclosure provides an opto-electronic device including: a first electrode; a second electrode facing the first electrode; and a photoactive layer arranged between the first electrode and the second electrode, wherein the photoactive layer includes a first layer and a second layer, the first layer including a first compound, and the second layer including a second compound that does not include a 5-membered ring, the first compound is an electron-donating compound, the second compound is an electron-accepting compound, and the opto-electronic device satisfies Equation 1. Equation 1 will be described below. A “photoactive” layer, as used herein, is responsive to light or another form of electromagnetic radiation.

In an embodiment, the first layer may be arranged between the first electrode and the second layer. For example, the first layer may be adjacent to the first electrode, and the second layer may be adjacent to the second electrode.

In an embodiment, the first layer may be in contact with the second layer.

In an embodiment, the opto-electronic device may further include a hole transport region arranged between the first electrode and the photoactive layer and an electron transport region arranged between the photoactive layer and the second electrode. The hole transport region may include a hole injection layer, a hole transport layer, an emission auxiliary layer, an electron-blocking layer, or any combination thereof. The electron transport region may include a buffer layer, a hole-blocking layer, an electron control layer, an electron transport layer, an electron injection layer, or any combination thereof.

In an embodiment, the first layer may be in contact with the hole transport region.

In an embodiment, the second layer may be in contact with the electron transport region.

In an embodiment, the photoactive layer may be formed by vacuum deposition.

Another aspect of the disclosure provides an electronic apparatus including the opto-electronic device.

In an embodiment, the electronic apparatus may further include a light-emitting device adjacent to the opto-electronic device. The light-emitting device may not overlap the opto-electronic device.

For example, light emitted by the light-emitting device may be extracted to the outside of the electronic apparatus. The light may be reflected by an external object to be incident into the electronic apparatus. The opto-electronic device may absorb the incident light. That is, the opto-electronic device may be used as a sensor that recognizes an object outside the electronic apparatus.

In an embodiment, the light-emitting device may include a first electrode, a second electrode facing the first electrode, and an interlayer arranged between the first electrode and the second electrode and including an emission layer. In an embodiment, the first electrode of the light-emitting device may be a portion of the first electrode of the opto-electronic device. In one or more embodiments, the first electrode of the light-emitting device may be apart from the first electrode of the opto-electronic device, but may include the same material as the first electrode of the opto-electronic device. In an embodiment, the second electrode of the light-emitting device may be a portion of the second electrode of the opto-electronic device. In one or more embodiments, the second electrode of the light-emitting device may be apart from the second electrode of the opto-electronic device, but may include the same material as the second electrode of the opto-electronic device. The emission layer may include a dopant and a host, and may emit light.

The term “interlayer” as used herein refers to a single layer and/or all of a plurality of layers arranged between the first electrode and the second electrode of the light-emitting device.

In an embodiment, the interlayer may further include a hole transport region arranged between the first electrode and the emission layer and an electron transport region arranged between the emission layer and the second electrode. The hole transport region may include a hole injection layer, a hole transport layer, an emission auxiliary layer, an electron-blocking layer, or any combination thereof. The electron transport region may include a buffer layer, a hole-blocking layer, an electron control layer, an electron transport layer, an electron injection layer, or any combination thereof.

For example, the first electrode, the hole transport region, the electron transport region, and the second electrode included in the opto-electronic device may be substantially identical to or different from the first electrode, the hole transport region, the electron transport region, and the second electrode included in the light-emitting device, respectively. That is, a portion of the opto-electronic device may extend to constitute a portion of the light-emitting device.

In an embodiment, the electronic apparatus may further include: a thin-film transistor electrically connected to the first electrode; and a color filter, a color conversion layer, a touch screen layer, a polarizing layer, or any combination thereof.

Another aspect of the disclosure provides an electronic device including the electronic apparatus, wherein the electronic device is one of a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, signage, and a billboard.

Hereinafter, Equation 1 satisfied by the opto-electronic device will be described:

0.1≤(E¹_{LUMO}−E²_{LUMO})/(E¹_{LUMO}−E¹_{HOMO})≤0.5 Equation 1

wherein, in Equation 1,
E¹_{LUMO} indicates a lowest unoccupied molecular orbital (LUMO) energy level of the first compound,
E¹_{HOMO} indicates a highest occupied molecular orbital (HOMO) energy level of the first compound, and
E²_{LUMO} indicates a LUMO energy level of the second compound.

In an embodiment, the first compound may be different from the second compound.

In an embodiment, E¹_{LUMO} may be greater than E²_{LUMO}, and E¹_{LUMO} may be greater than E¹_{HOMO}.

In an embodiment, the opto-electronic device may satisfy Equation 1-1:

0.12≤(E¹_{LUMO}−E²_{LUMO})/(E¹_{LUMO}−E¹_{HOMO})≤0.45. Equation 1-1

For example, a value of [(E¹_{LUMO}−E²_{LUMO})/(E¹_{LUMO}−E¹_{HOMO})] may be in a range of about 0.14 to about 0.44. In an embodiment, the value of [(E¹_{LUMO}−E²_{LUMO})/(E¹_{LUMO}−E¹_{HOMO})] may be in a range of about 0.17 to about 0.43. In one or more embodiments, the value of [(E¹_{LUMO}−E²_{LUMO})/(E¹_{LUMO}−E¹_{HOMO})] may be in a range of about 0.29 to about 0.4.

In an embodiment, the opto-electronic device may satisfy Equation 2:

0.25 eV≤E¹_{LUMO}−E²_{LUMO}. Equation 2

For example, a value of [E¹_{LUMO}−E²_{LUMO}] may be in a range of about 0.3 eV to about 2 eV, for example, about 0.45 eV to about 1.5 eV.

In an embodiment, the opto-electronic device may satisfy Equation 3:

E²_{HOMO}≤E¹_{HOMO} Equation 3

wherein, in Equation 3,
E²_{HOMO} indicates a HOMO energy level of the second compound.

In an embodiment, the opto-electronic device may satisfy Equation 4:

3 eV≤E²_{LUMO}−E²_{HOMO}≤4 eV. Equation 4

For example, a value of [E²_{LUMO}−E²_{HOMO}] may be in a range of about 3.4 eV to about 3.7 eV.

In an embodiment, a maximum absorption wavelength of the first compound may be in a range of about 500 nm to about 600 nm. For example, the first compound may absorb green light.

In an embodiment, the first compound may not include fullerene. For example, the first compound may not include fullerene 60, and may not include fullerene 70.

In an embodiment, the first compound may not include a 4-membered ring. For example, the first compound may not include a ring formed by bonding of four atoms selected from C, N, O, S, and the like.

In an embodiment, the first compound may be selected from Compounds D1 to D4:

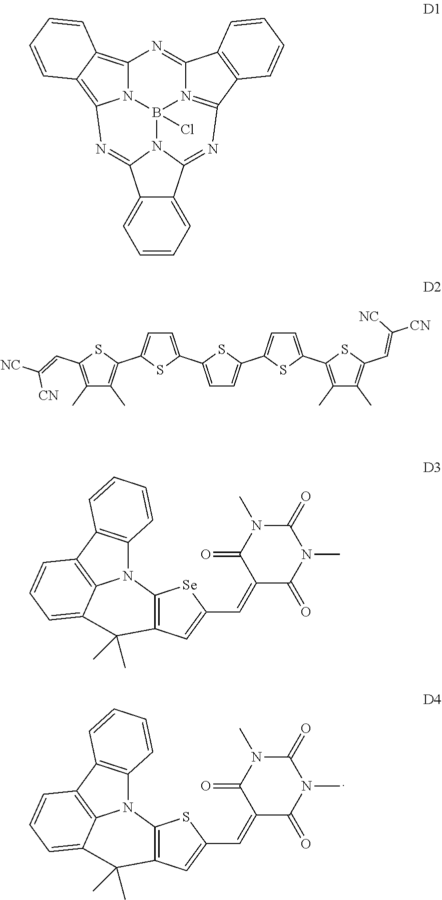

In an embodiment, the second compound may be represented by Formula 1:

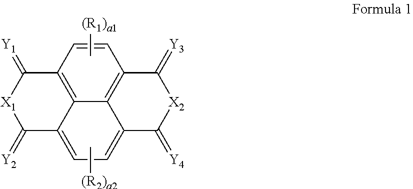

wherein, in Formula 1,
X₁ may be O or N(Ar₁),
X₂ may be O or N(Ar₂),
Ar₁ and Ar₂ may each independently be hydrogen, deuterium, a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Y₁ may be O or C(R₁₁)(R₁₂),
Y₂ may be O or C(R₂₁)(R₂₂),
Y₃ may be O or C(R₃₁)(R₃₂),
Y₄ may be O or C(R₄₁)(R₄₂),
a1 and a2 may each independently be 0, 1, or 2,
R₁, R₂, R₁₁, R₁₂, R₂₁, R₂₂, R₃₁, R₃₂, R₄₁, and R₄₂ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, X₁ may be O, and X₂ may be O.

In an embodiment, when X₁ and X₂ are each O, neither R₁ nor R₂ may be —F, —Cl, —Br, —I, or a cyano group. For example, when X₁ and X₂ are each O, R₁ and R₂ may each independently be hydrogen or deuterium.

In an embodiment, X₁ may be N(Ar₁), X₂ may be N(Ar₂), and Ar₁ and Ar₂ may each independently be a C₁-C₆₀ alkyl group unsubstituted or substituted with at least one R₁₀ₐ or a C₆-C₆₀ aryl group unsubstituted or substituted with at least one R₁₀ₐ.

For example, Ar₁ and Ar₂ may each independently be a C₁-C₄ alkyl group unsubstituted or substituted with at least one R₁₀ₐ or a phenyl group unsubstituted or substituted with at least one R₁₀ₐ.

In an embodiment, Y₁ to Y₄ may each be O.

In an embodiment, the second compound may not include S.

In an embodiment, the second compound may be one of Compounds A1 to A9:

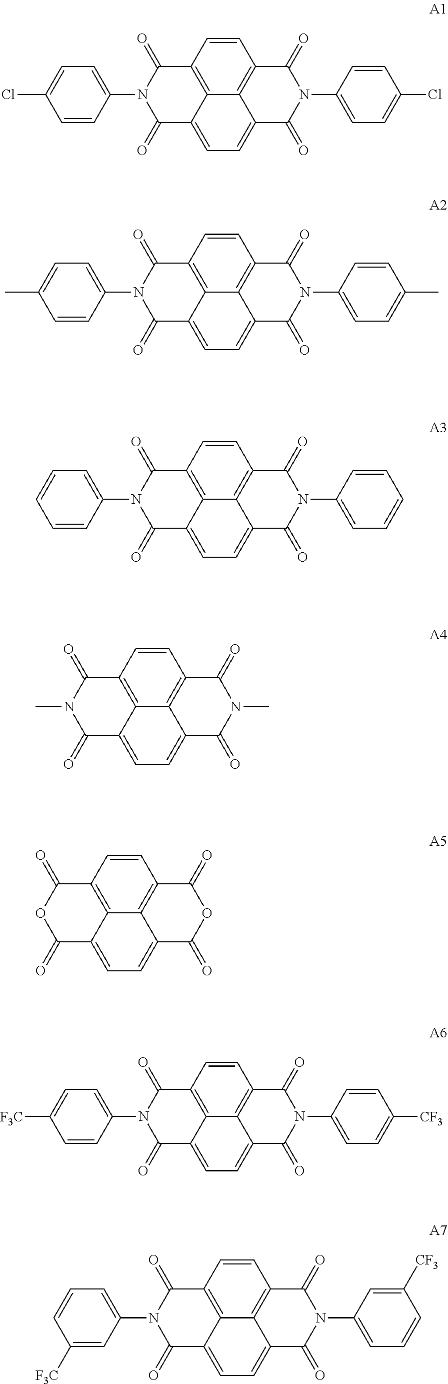

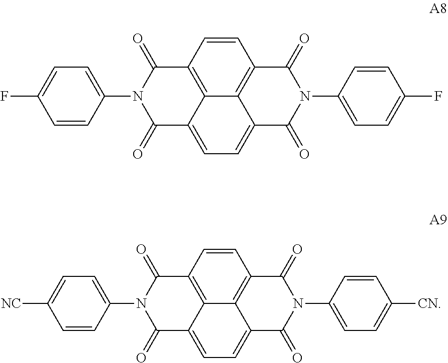 [SMILES: O=C1c2ccc3c4c(ccc(c24)C(=O)N1c1ccc(F)cc1)C(=O)N(c1ccc(F)cc1)C3=O ; N#Cc1ccc(N2C(=O)c3ccc4c5c(ccc(c35)C2=O)C(=O)N(c2ccc(C#N)cc2)C4=O)cc1]

The first compound has an appropriate LUMO energy level and an appropriate HOMO energy level so as to have a maximum absorption wavelength in a range of about 500 nm to about 600 nm. The second compound does not include a 5-membered ring, has an appropriate LUMO energy level so as to have excellent charge-transfer characteristics with the first compound, and has an appropriate LUMO energy level so as to increase excitons generated by the first compound. That is, in an opto-electronic device including the first compound and the second compound, a value of [(E¹_{LUMO}−E²_{LUMO})/(E¹_{LUMO}−E¹_{HOMO})] satisfies a range of about 0.1 to about 0.5. Thus, as the optimal charge balance is controlled, charge-transfer characteristics and exciton-forming characteristics of the opto-electronic device may be improved. Accordingly, the opto-electronic device may have excellent photoelectric characteristics.

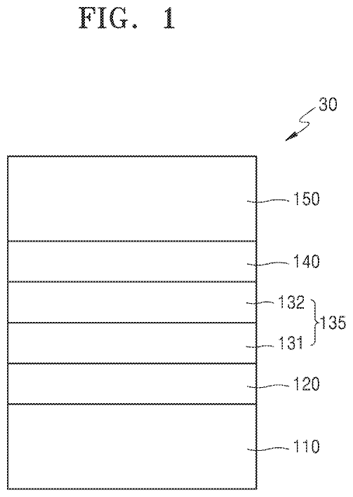

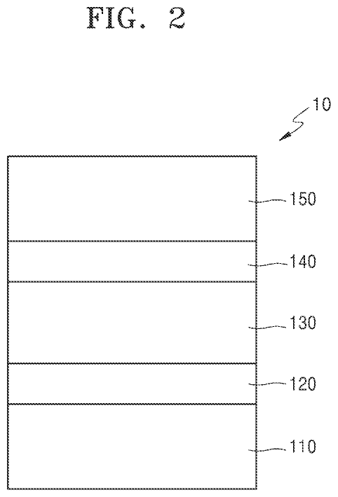

### Description of FIGS. 1 and 2

FIG. 1 is a schematic view of an opto-electronic device 30 according to an embodiment. The opto-electronic device 30 may include a first electrode 110, a hole transport region 120, a photoactive layer 135, an electron transport region 140, and a second electrode 150.

FIG. 2 is a schematic view of a light-emitting device 10. The light-emitting device 10 may include a first electrode 110, a hole transport region 120, an emission layer 130, an electron transport region 140, and a second electrode 150.

In an embodiment, the first electrode 110, the hole transport region 120, the electron transport region 140, and the second electrode 150 of the opto-electronic device 30 may have a substantially single body with the first electrode 110, the hole transport region 120, the electron transport region 140, and the second electrode 150 of the light-emitting device 10, respectively. In one or more embodiments, the first electrode 110, the hole transport region 120, the electron transport region 140, and the second electrode 150 of the opto-electronic device 30 may be apart from the first electrode 110, the hole transport region 120, the electron transport region 140, and the second electrode 150 of the light-emitting device 10, respectively, but may include substantially the same material and be formed substantially at the same time as the first electrode 110, the hole transport region 120, the electron transport region 140, and the second electrode 150 of the light-emitting device 10, respectively.

Hereinafter, the structures of the opto-electronic device 30 and the light-emitting device 10 according to embodiments and methods of manufacturing the opto-electronic device 30 and the light-emitting device 10 will be described with reference to FIGS. 1 and 2.

[First Electrode 110]

In FIG. 1, a substrate may be additionally arranged under the first electrode 110 or on the second electrode 150. As the substrate, a glass substrate or a plastic substrate may be used. In one or more embodiments, the substrate may be a flexible substrate, and may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

[Hole Transport Region 120]

The hole transport region 120 may have i) a single-layered structure consisting of a single layer consisting of a single material, ii) a single-layered structure consisting of a single layer including a plurality of different materials, or iii) a multi-layered structure including a plurality of layers including different materials.

The hole transport region 120 may include a hole injection layer, a hole transport layer, an emission auxiliary layer, an electron-blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the layers of each structure being stacked sequentially from the first electrode 110.

The hole transport region 120 may include a compound represented by Formula 201, a compound represented by Formula 202, or any combination thereof:

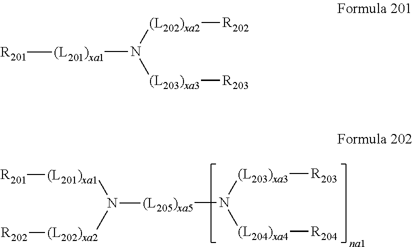

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S*′, *—N(Q₂₀₁)*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16, etc.),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

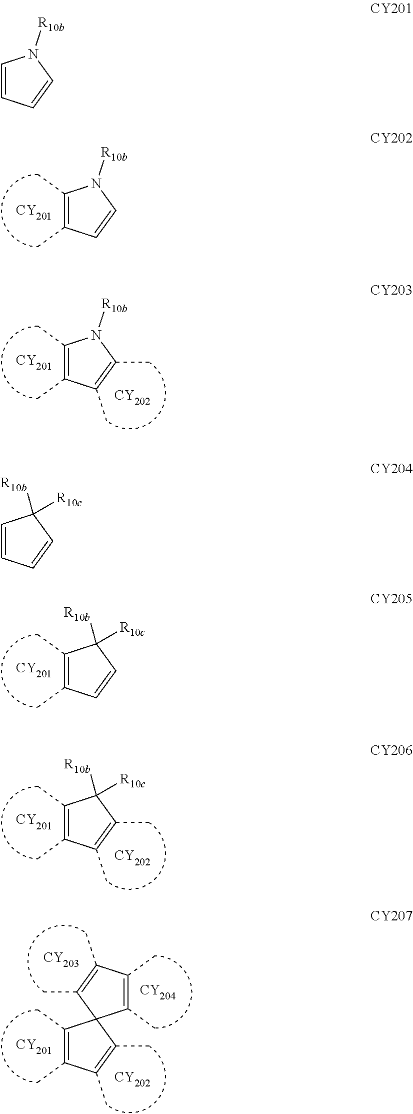

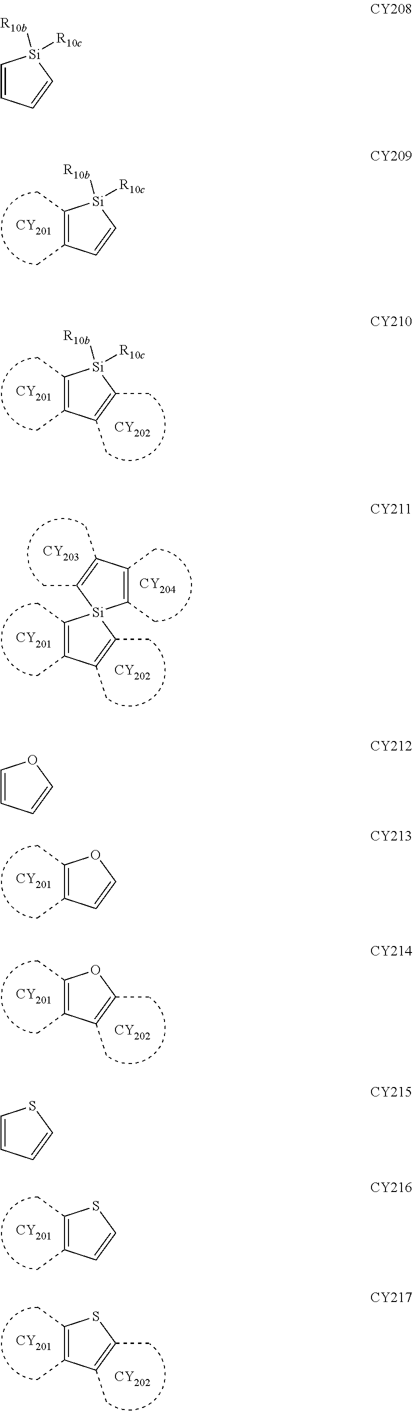

wherein, in Formulae CY201 to CY217, R_{10b} and R_{10c} may each be as defined herein in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as defined herein.

In an embodiment, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of groups represented by Formulae CY201 to CY203.

In one or more embodiments, Formula 201 may include at least one of groups represented by Formulae CY201 to CY203 and at least one of groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include a group represented by one of Formulae CY201 to CY203.

In one or more embodiments, each of Formulae 201 and 202 may not include a group represented by one of Formulae CY201 to CY203, and may include at least one of groups represented by Formulae CY204 to CY217.

In one or more embodiments, each of Formulae 201 and 202 may not include a group represented by one of Formulae CY201 to CY217.

For example, the hole transport region 120 may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

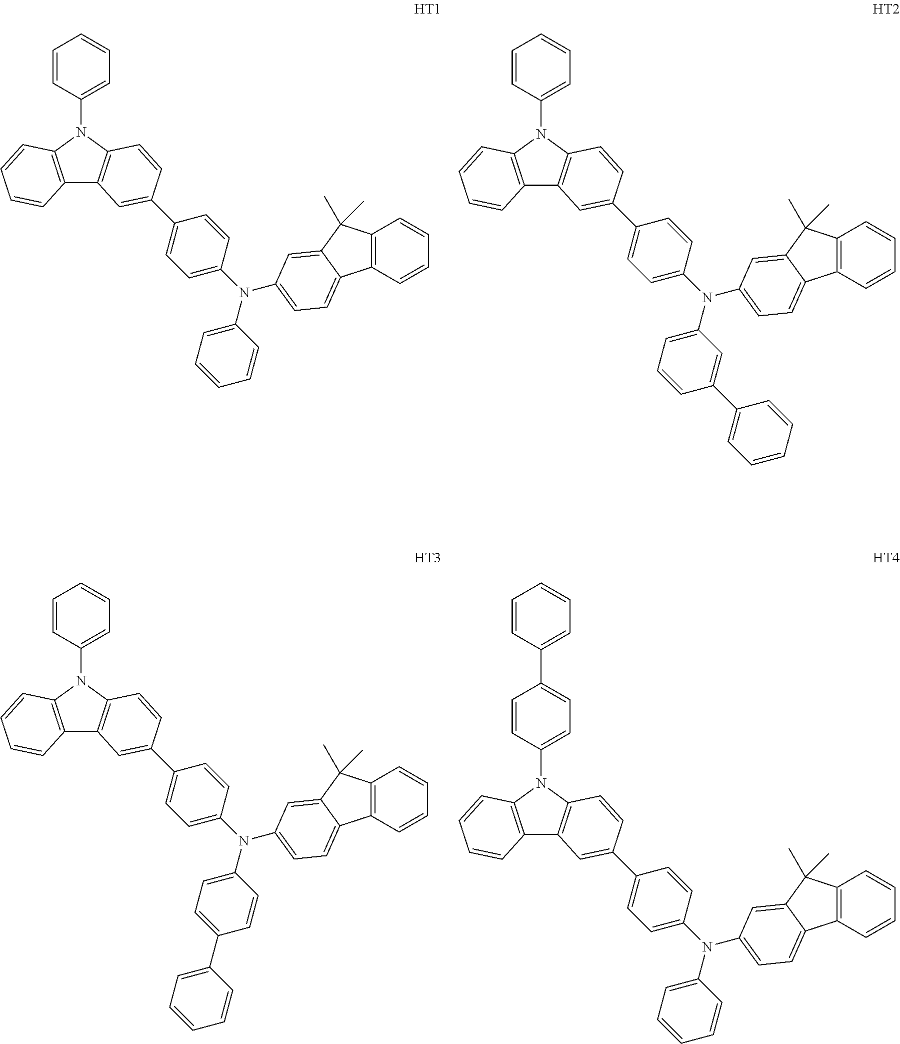

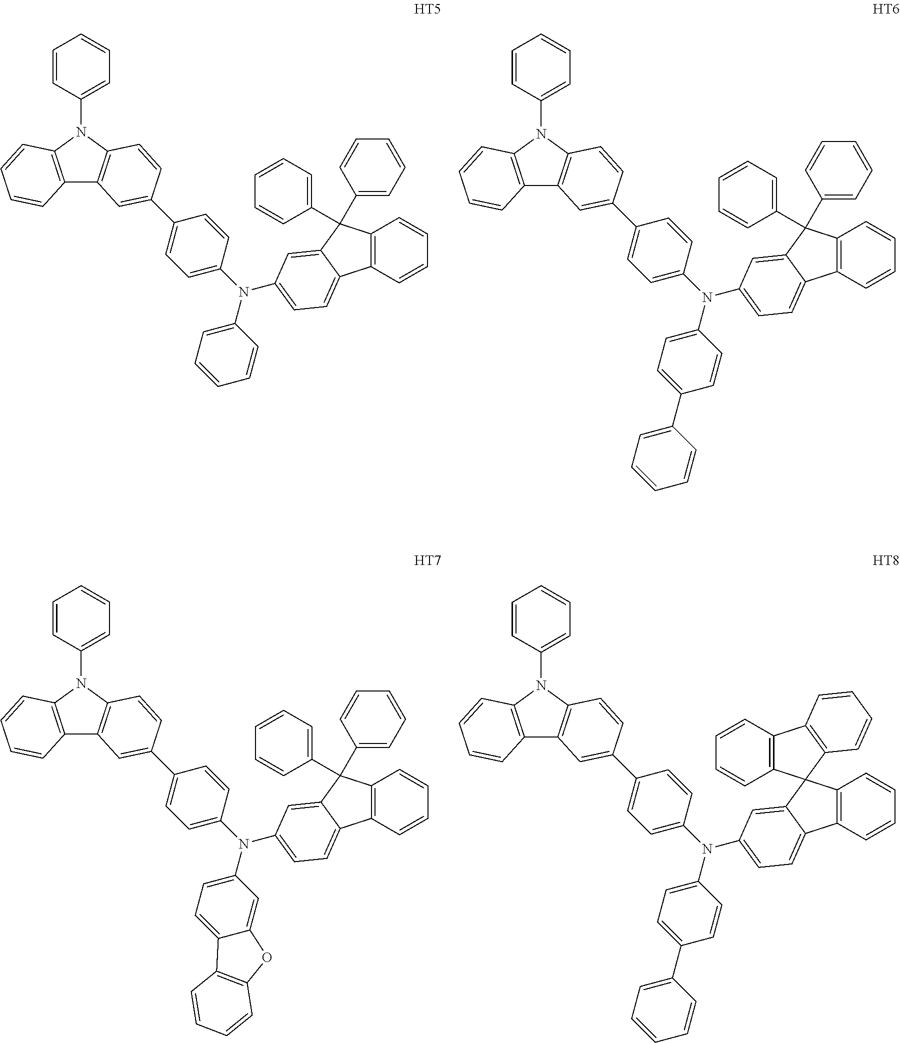 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

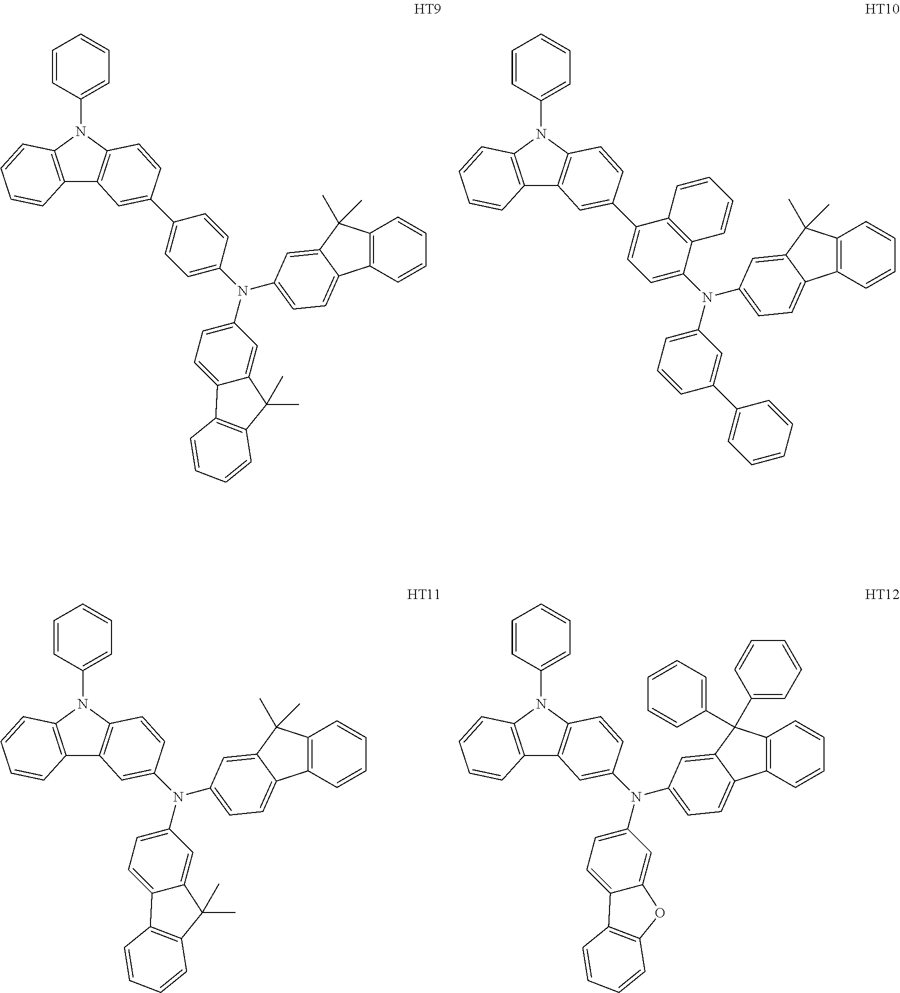 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

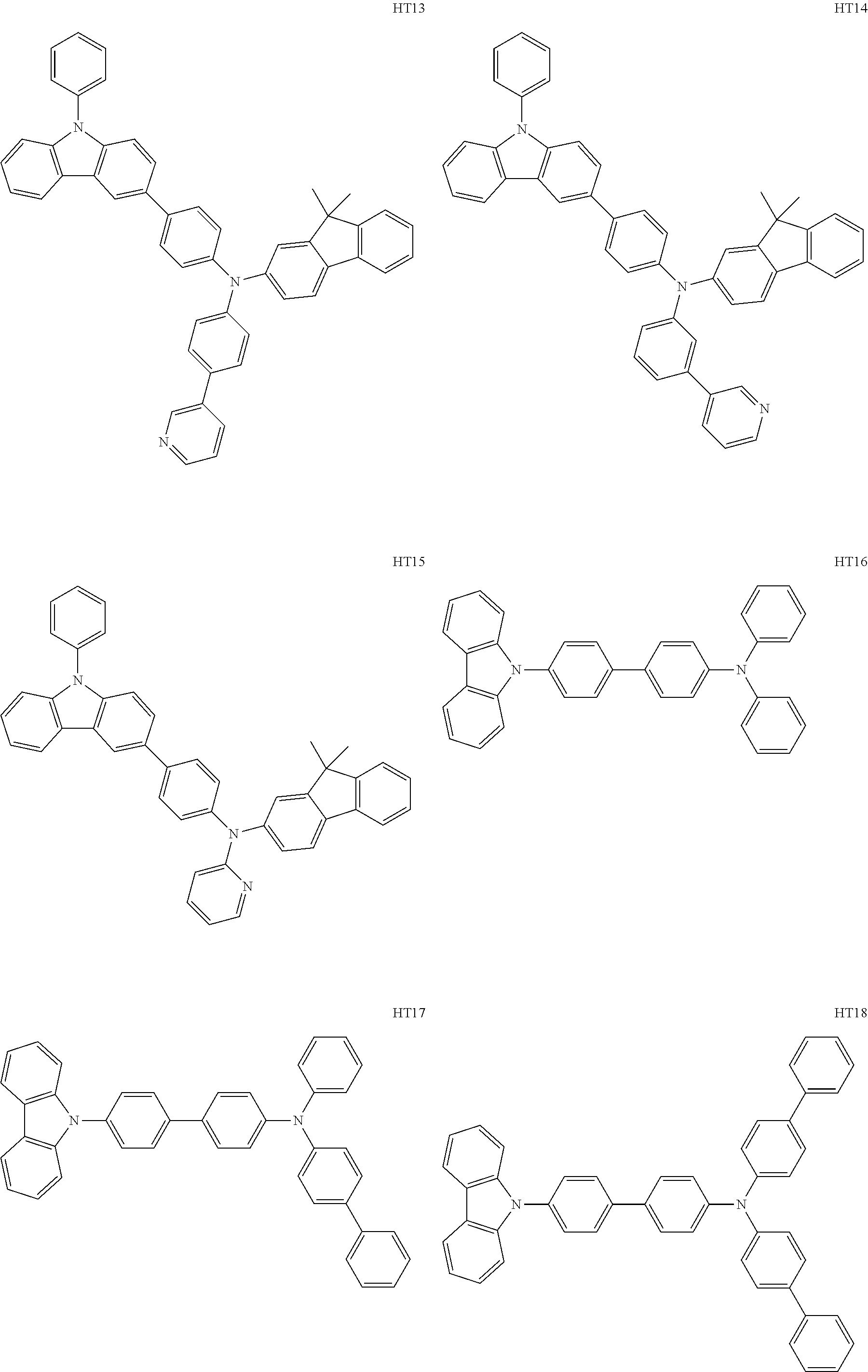

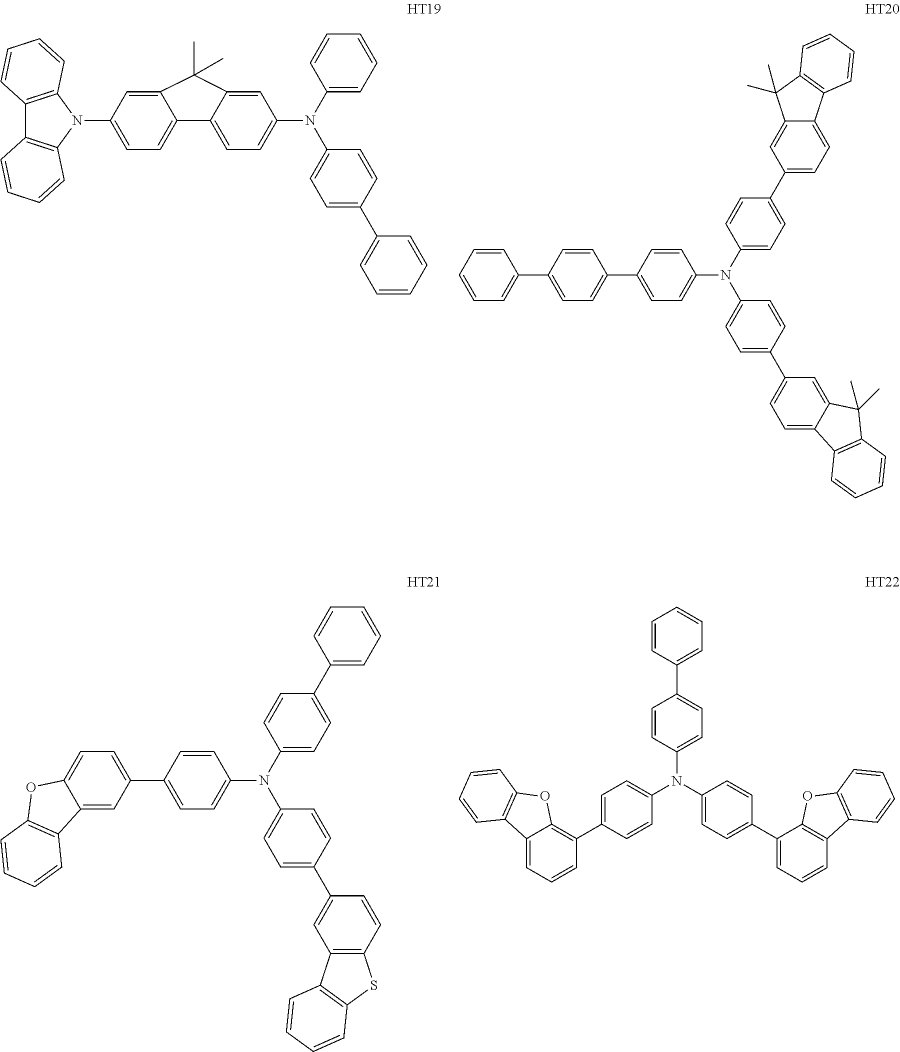

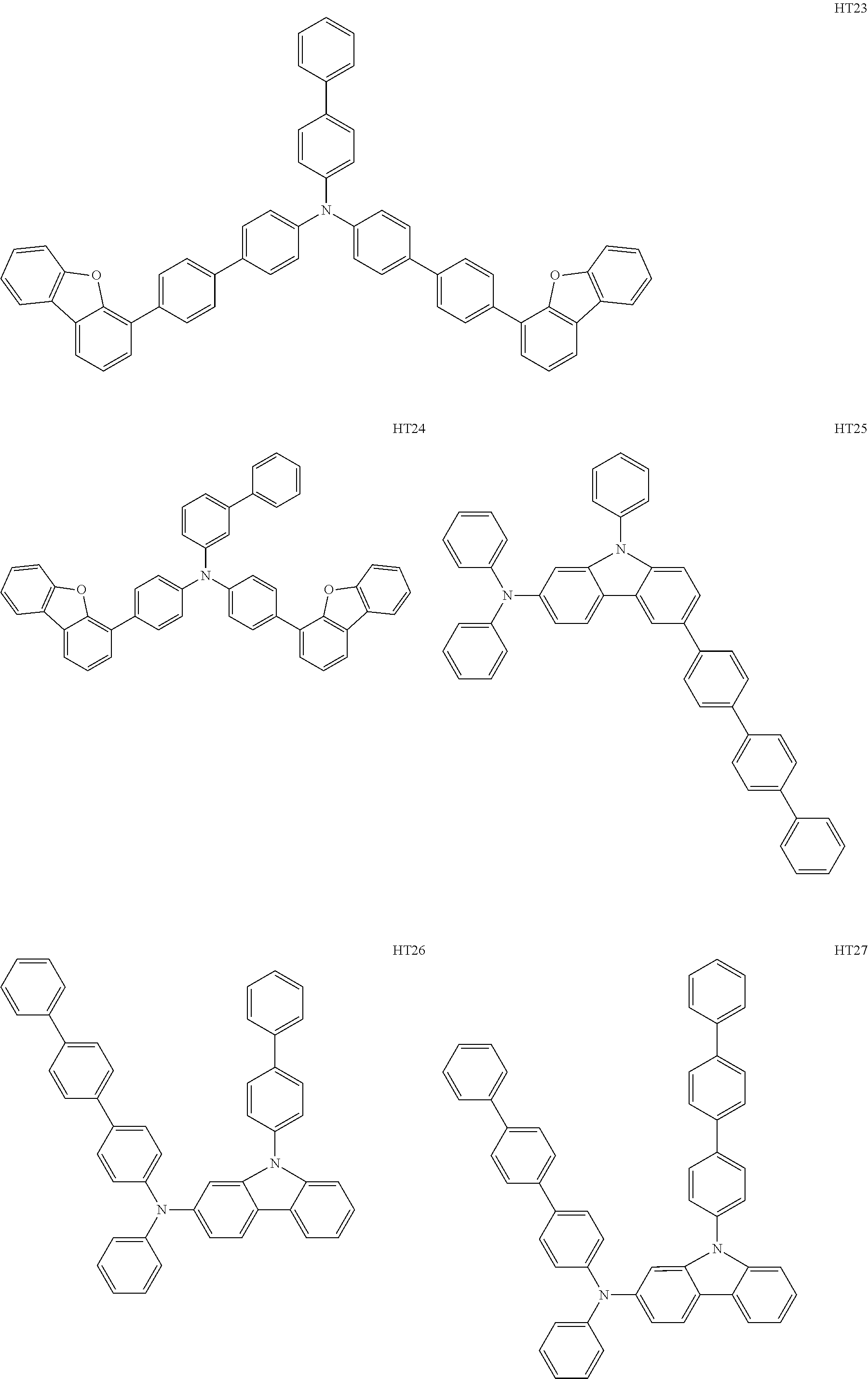 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(N(c4ccccc4)c4ccc5c6ccccc6n(-c6ccc(-c7ccc(-c8ccccc8)cc7)cc6)c5c4)cc3)cc2)cc1]

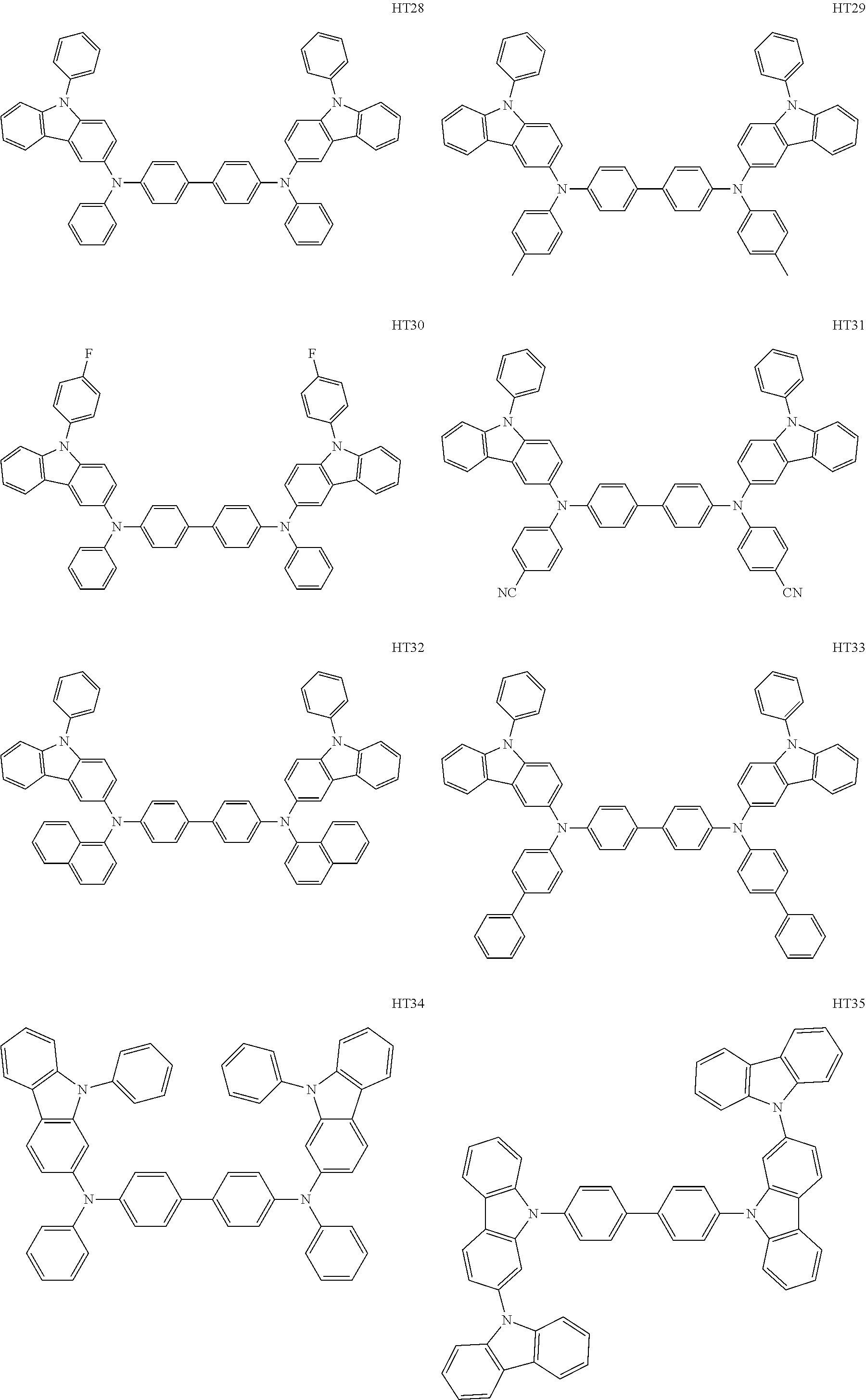

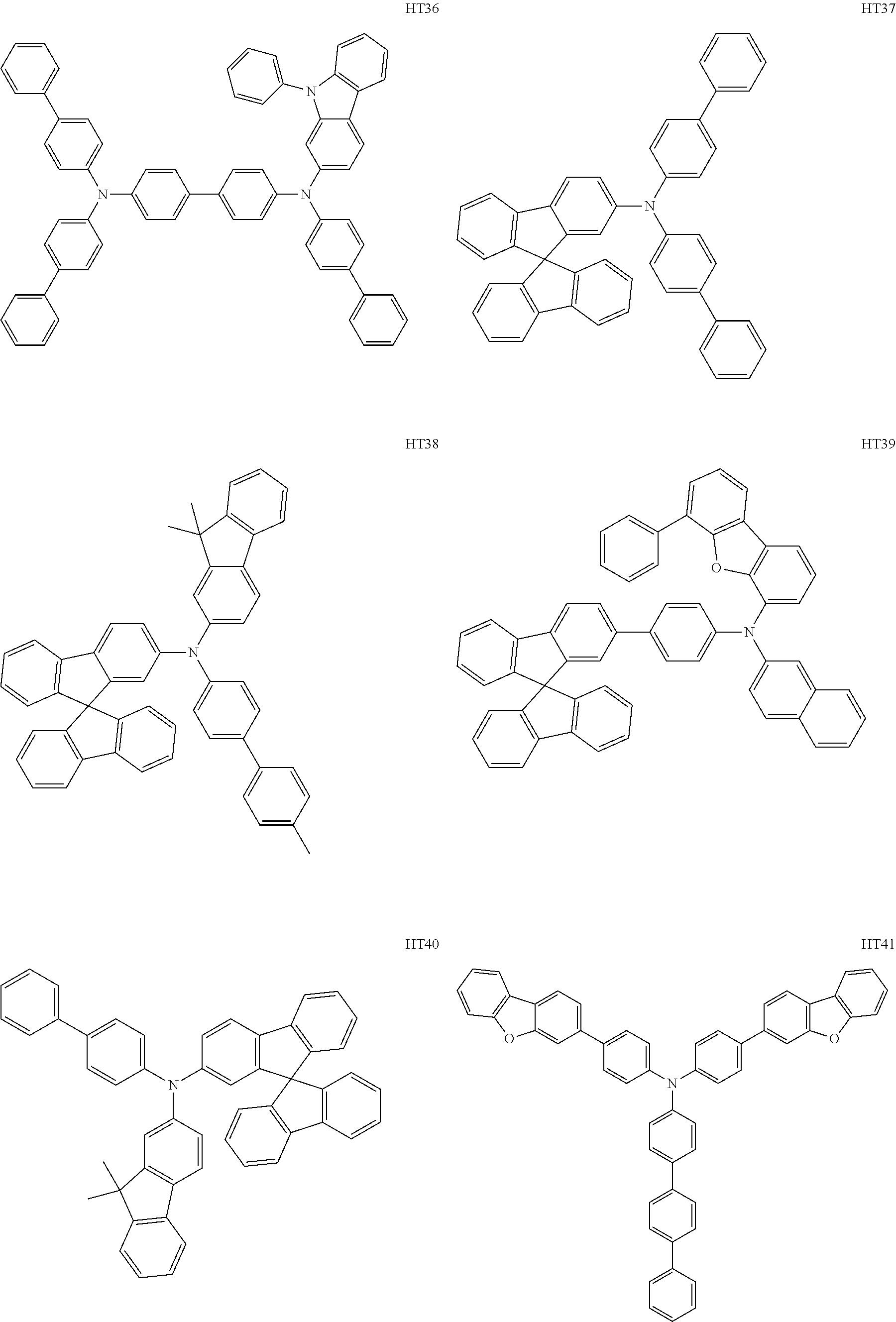

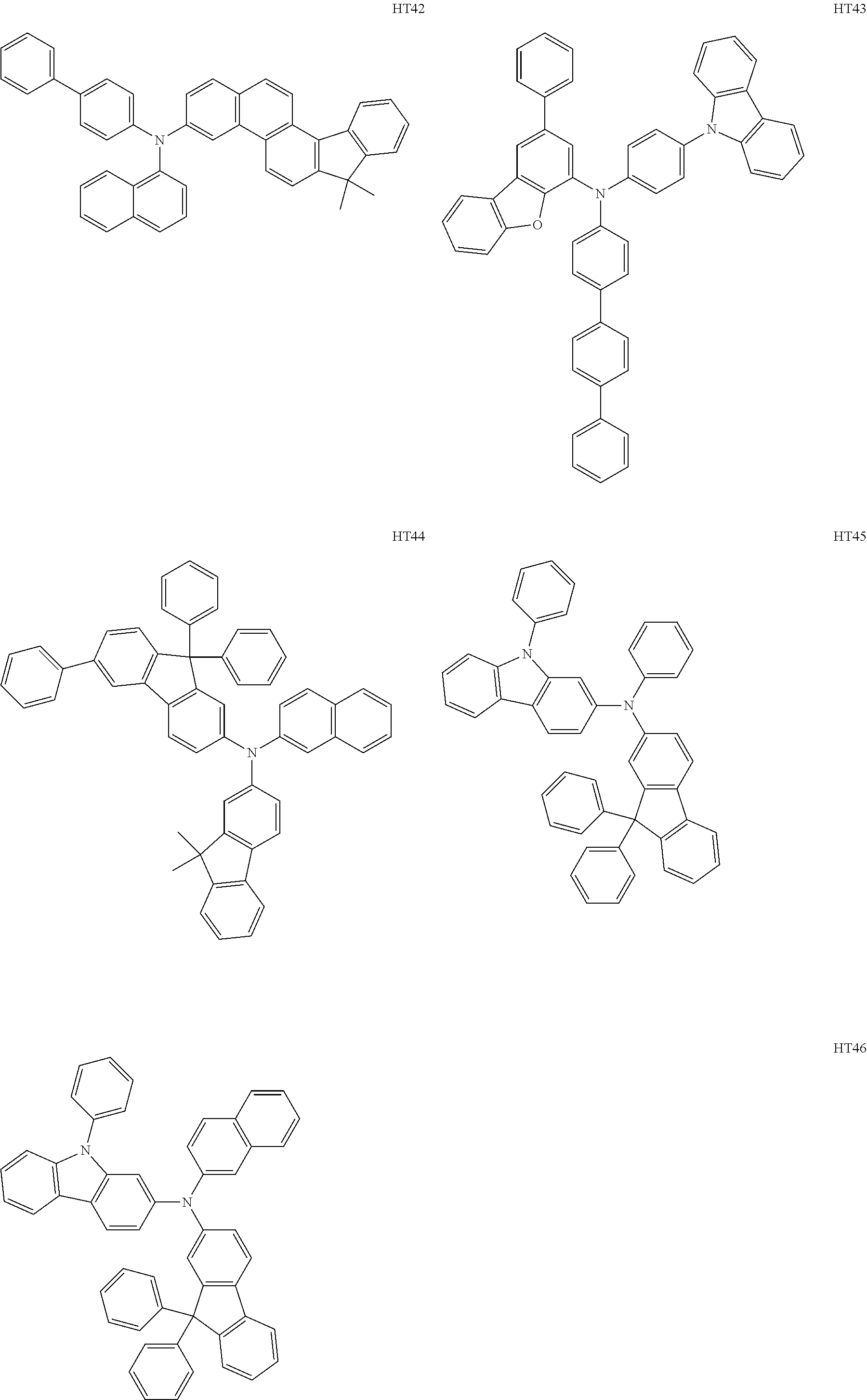

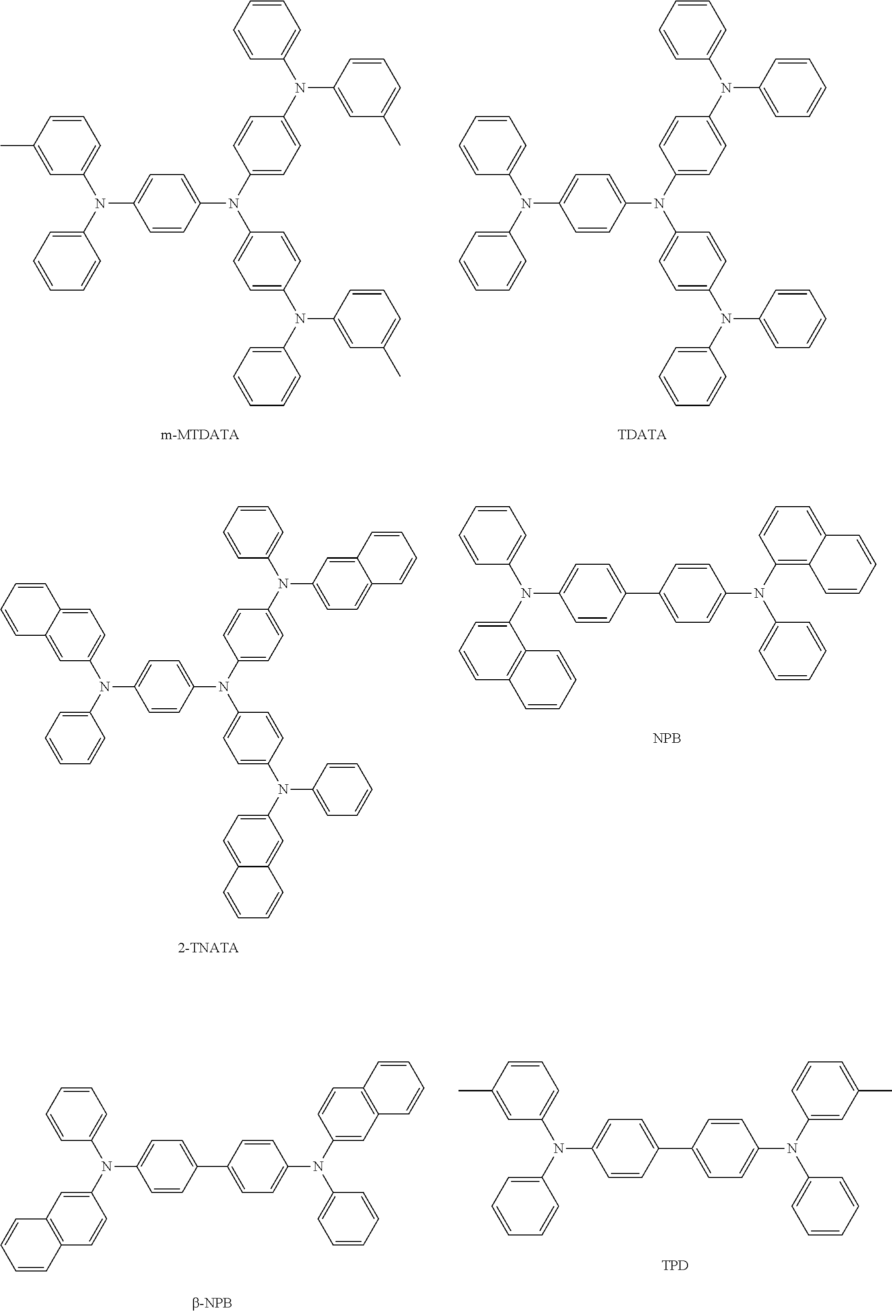

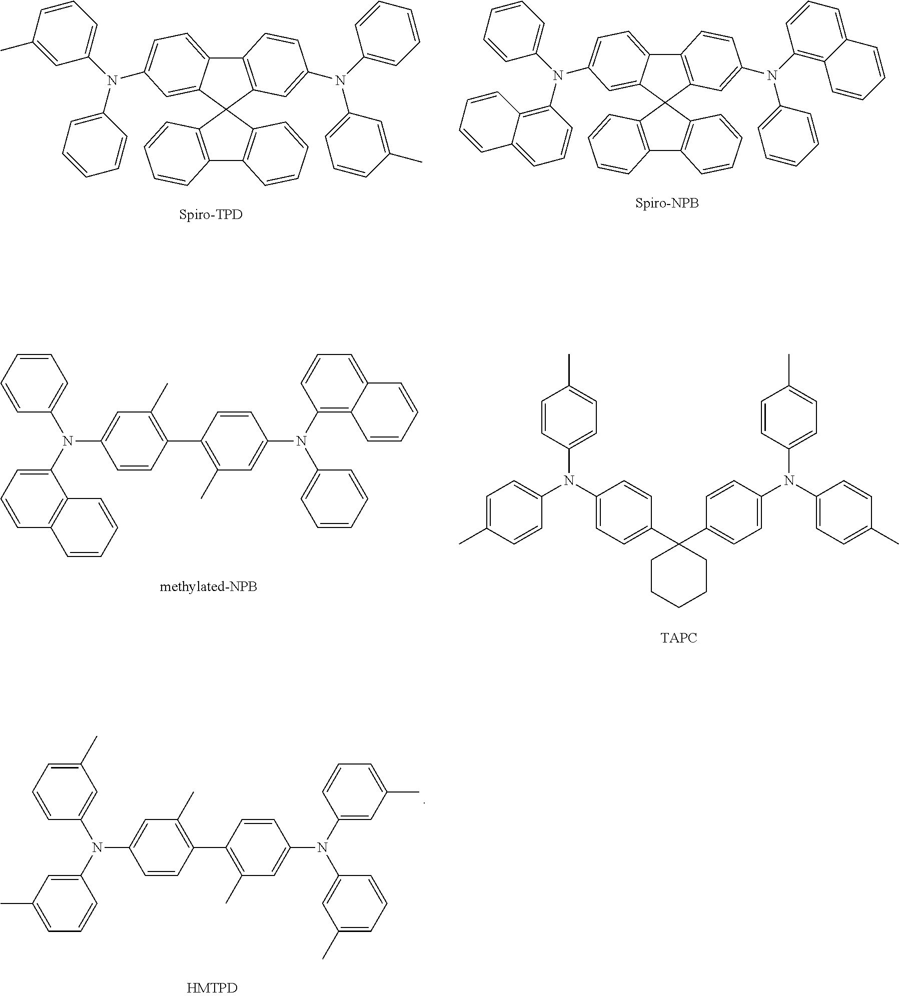

A thickness of the hole transport region 120 may be in a range of about 50 Å to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within these ranges, satisfactory hole-transporting characteristics may be obtained without a substantial increase in driving voltage.

The emission auxiliary layer may increase light-emission efficiency by compensating for an optical resonance distance according to the wavelength of light emitted by the emission layer 130, and the electron-blocking layer may block the leakage of electrons from the emission layer 130 to the hole transport region 120. Materials that may be included in the hole transport region 120 may be included in the emission auxiliary layer and the electron-blocking layer.

[p-Dopant]

The hole transport region 120 may further include, in addition to the materials as described above, a charge-generation material for improving conductive properties. The charge-generation material may be uniformly or non-uniformly dispersed in the hole transport region 120 (e.g., in the form of a single layer consisting of a charge-generation material).

The charge-generation material may be, for example, a p-dopant.

For example, a LUMO energy level of the p-dopant may be −3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

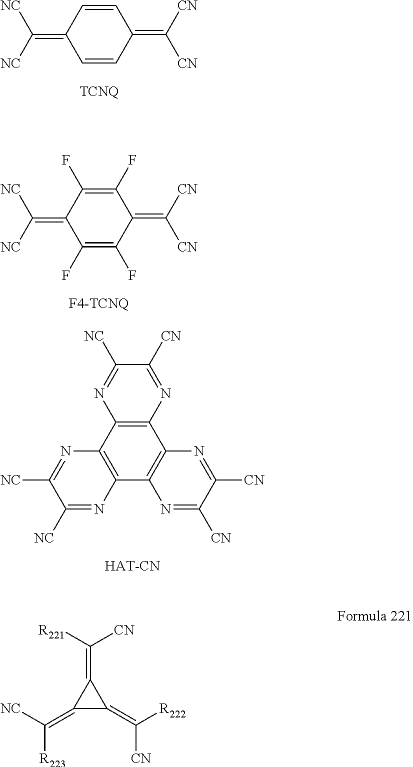

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of the metalloid may include silicon (Si), antimony (Sb), tellurium (Te), and the like.

Examples of the non-metal may include oxygen (O), halogen (e.g., F, Cl, Br, I, etc.), and the like.

Examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, or any combination thereof.

Examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (e.g., MoO, Mo₂O₃, MoO₂, MoO₃, Mo₂O₅, etc.), a rhenium oxide (e.g., ReO₃, etc.), and the like.

Examples of the metal halide may include an alkali metal halide, an alkaline earth metal halide, a transition metal halide, a post-transition metal halide, a lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include a titanium halide (e.g., TiF₄, TiCl₄, TiBr₄, TiI₄, etc.), a zirconium halide (e.g., ZrF₄ₗ, ZrC₄, ZrBr₄, ZrI₄, etc.), a hafnium halide (e.g., HfF₄, HfCl₄, HfBr₄, HfI₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, TaI₃, etc.), a chromium halide (e.g., CrF₃, CrCl₃, CrBr₃, CrI₃, etc.), a molybdenum halide (e.g., MoF₃, MoCl₃, MoBr₃, MoI₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, Tcl₂, etc.), a rhenium halide (e.g., ReF₂, ReCl₂, ReBr₂, ReI₂, etc.), an iron halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, COCl₂, CoBr₂, CoI₂, etc.), a rhodium halide (e.g., RhF₂, RhCl₂, RhBr₂, RhI₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCI₂, PdBr₂, PdI₂, etc.), a platinum halide (e.g., PtF₂, PtCl₂, PtBr₂, PtI₂, etc.), a copper halide (e.g., CuF, CuCl, CuBr, CuI, etc.), a silver halide (e.g., AgF, AgCl, AgBr, Agl, etc.), a gold halide (e.g., AuF, AuCl, AuBr, Aul, etc.), and the like.

Examples of the post-transition metal halide may include a zinc halide (e.g., ZnF₂, ZnCl₂, ZnBr₂, ZnI₂, etc.), an indium halide (e.g., InI₃, etc.), a tin halide (e.g., SnI₂, etc.), and the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of the metalloid halide may include an antimony halide (e.g., SbCl₅, etc.) and the like.

Examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), a lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and the like.

[Emission Layer 130]

The light-emitting device 10 may include the emission layer 130 arranged on the hole transport region 120.

The emission layer 130 may further include, in addition to various organic materials, a metal-containing compound, such as an organometallic compound, an inorganic material, such as a quantum dot, and the like.

In an embodiment, the emission layer 130 may include i) two or more emitting units sequentially stacked between the first electrode 110 and the second electrode 150, and ii) a charge generation layer arranged between the two or more emitting units. When the emission layer 130 includes the emitting units and the charge generation layer as described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a subpixel. In an embodiment, the emission layer 130 may have a stacked structure of two or more layers of a red emission layer, a green emission layer, and a blue emission layer, in which the two or more layers contact each other or are separated from each other to emit white light. In one or more embodiments, the emission layer 130 may include two or more materials of a red light-emitting material, a green light-emitting material, and a blue light-emitting material, in which the two or more materials are mixed with each other in a single layer to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In an embodiment, the emission layer 130 may include a quantum dot.

In an embodiment, the emission layer 130 may include a delayed fluorescence material. The delayed fluorescence material may serve as a host or a dopant in the emission layer 130.

A thickness of the emission layer 130 may be in a range of about 100 Å to about 1,000 Å, for example, about 200 Å to about 600 Å. When the thickness of the emission layer 130 is within these ranges, excellent luminescence characteristics may be obtained without a substantial increase in driving voltage.

[Host]

The host may include a compound represented by Formula 301:

[Ar₃₀₁]_{xb11}-[(L₃₀₁)_{xb1}-R₃₀₁]_{xb21} Formula 301

wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be as defined herein in connection with Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

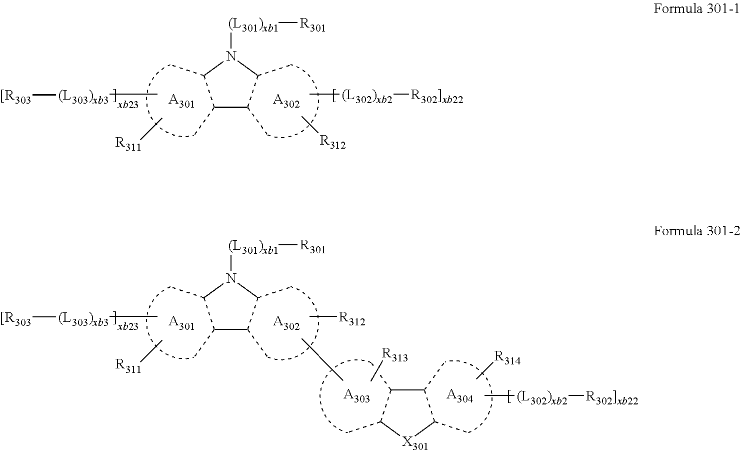

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be as defined herein,
L₃₀₂ to L₃₀₄ may each independently be as defined herein in connection with L₃₀₁,
xb2 to xb4 may each independently be as defined herein in connection with xb1, and
R₃₀₂ to R₃₀₅ and R₃₁₁ to R₃₁₄ may each be as defined herein in connection with R₃₀₁.

In one or more embodiments, the host may include an alkali earth metal complex, a post-transition metal complex, or any combination thereof. For example, the host may include a Be complex (e.g., Compound H55), a Mg complex, a Zn complex, or any combination thereof.

In one or more embodiments, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), or any combination thereof:

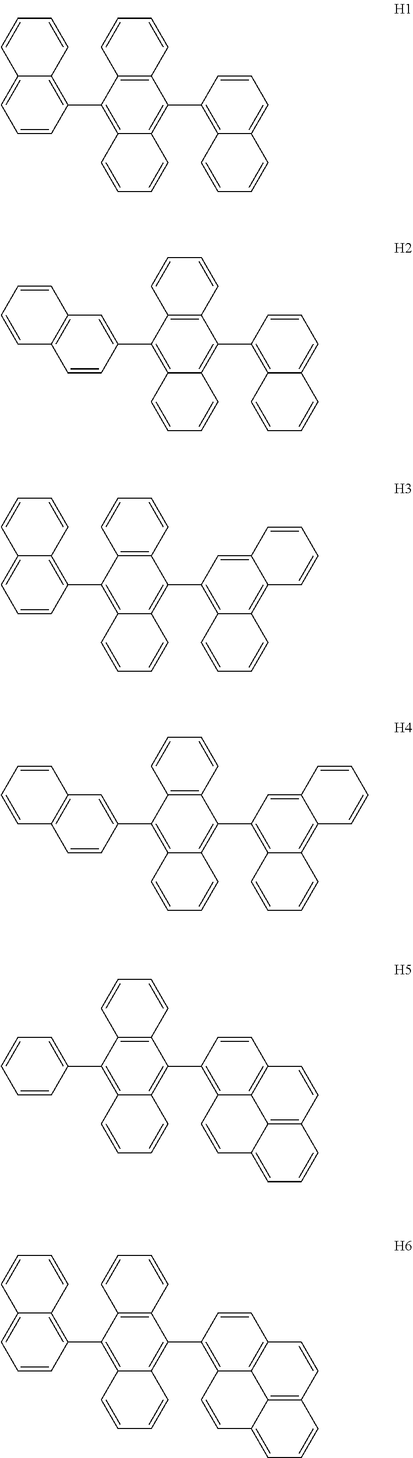

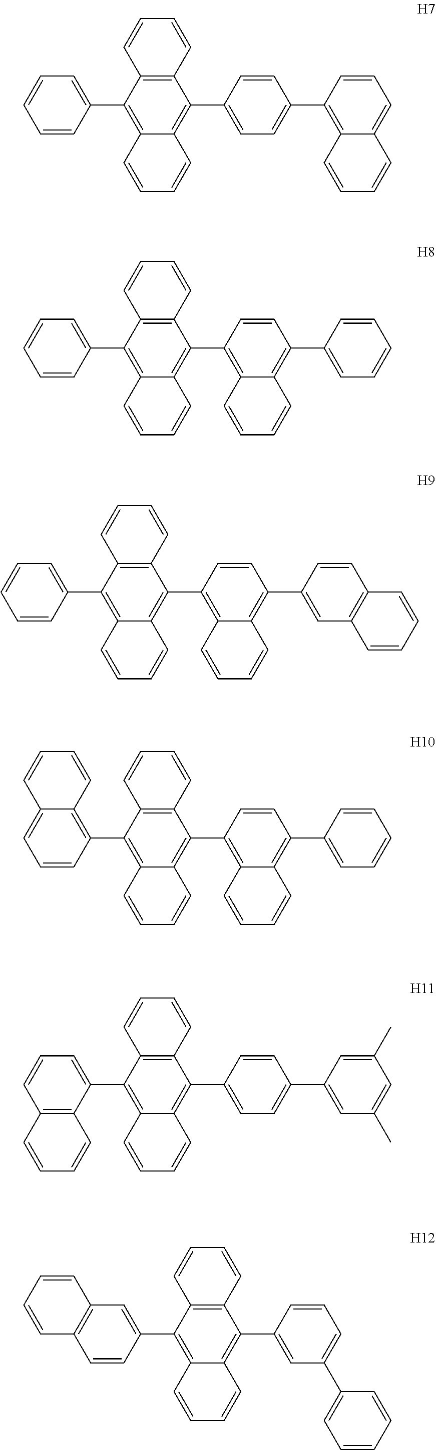

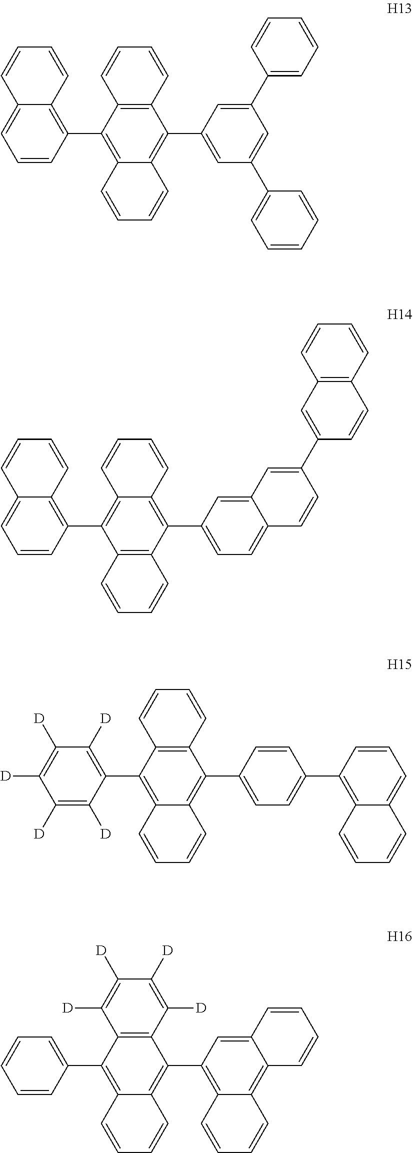

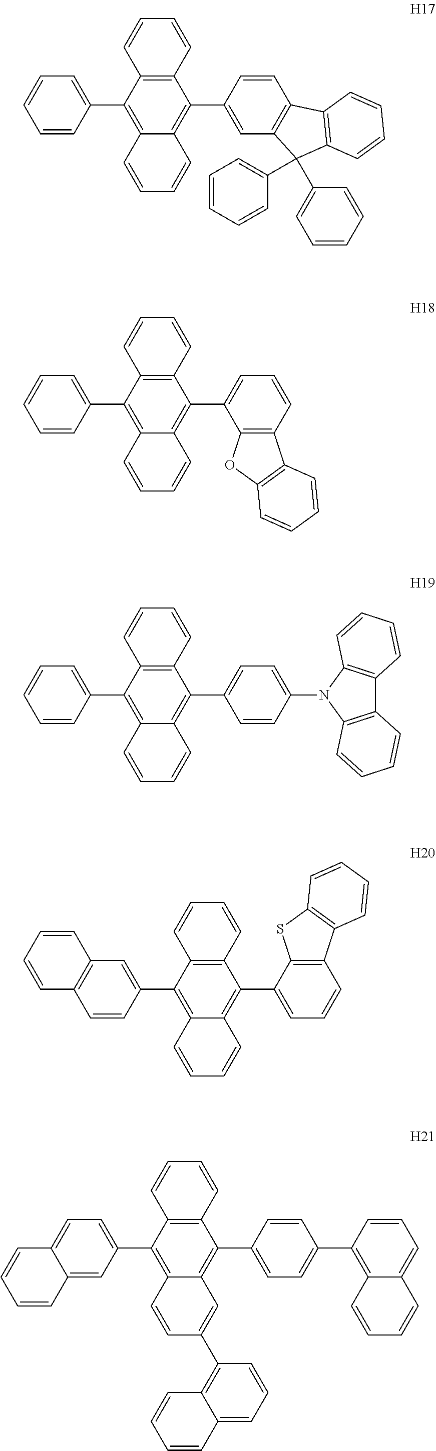

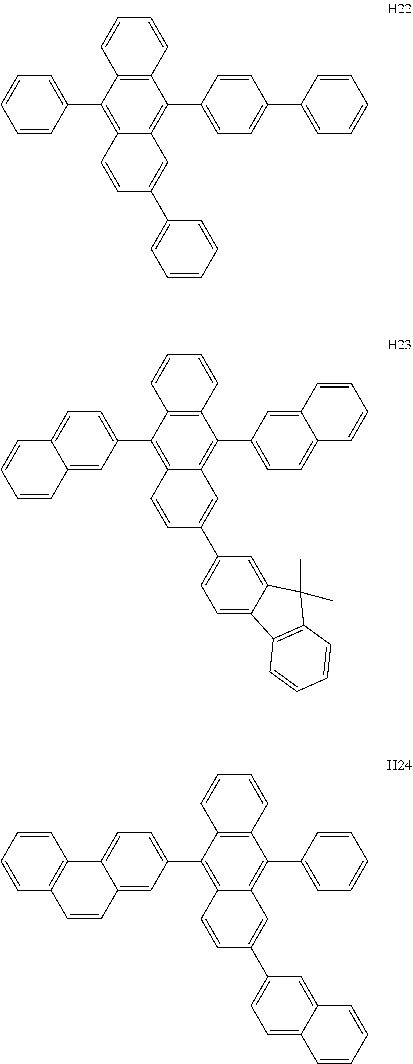

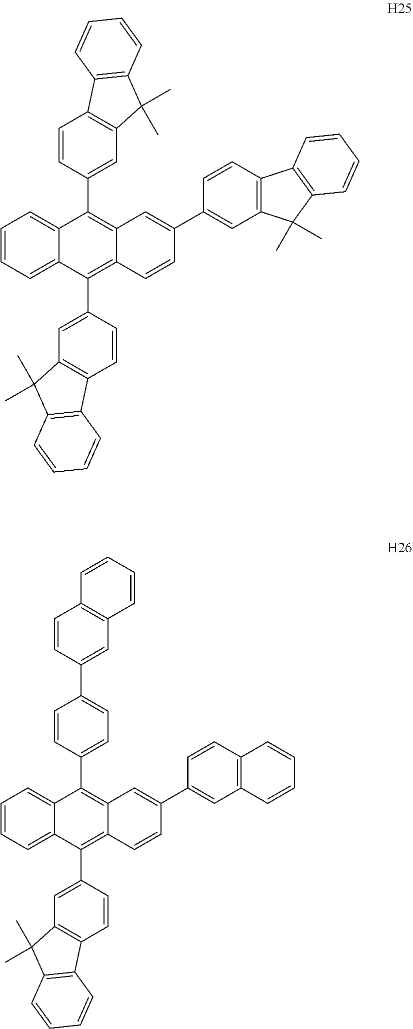

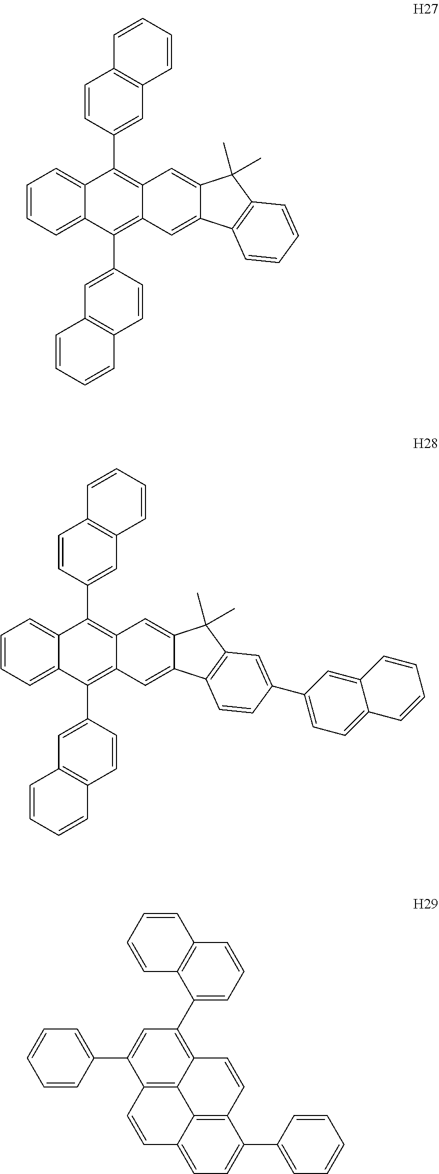

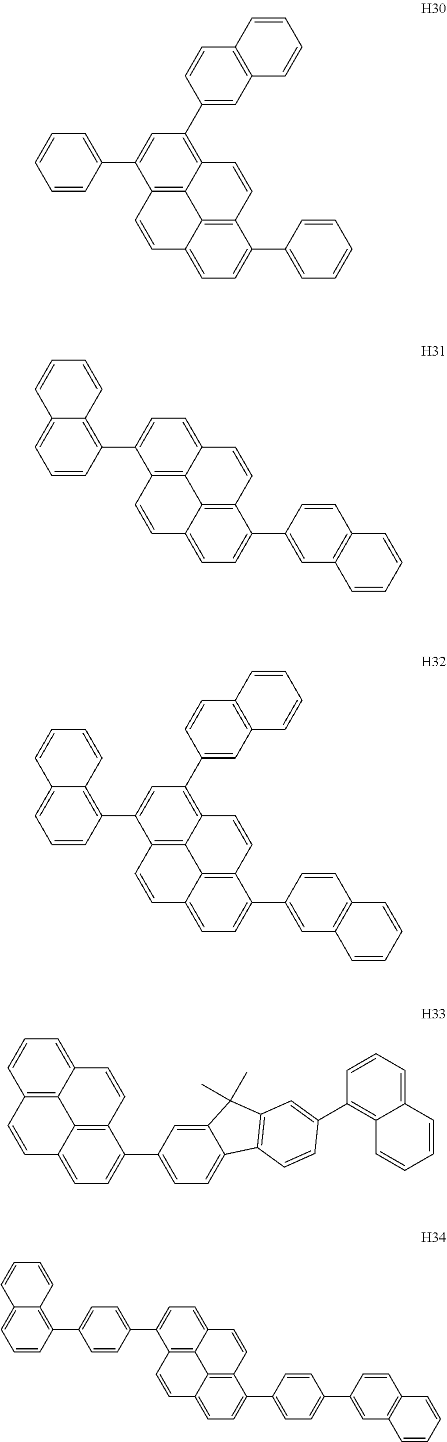

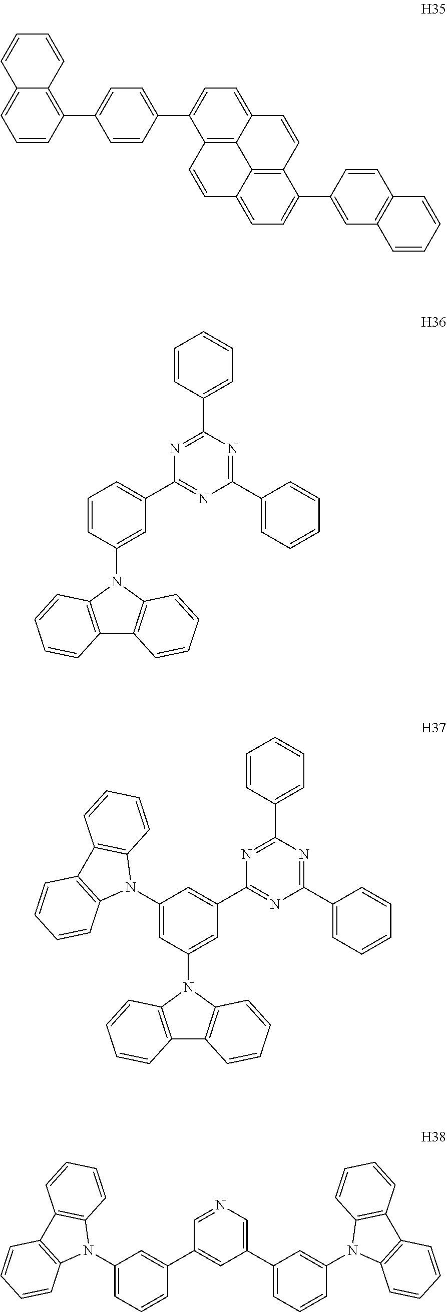

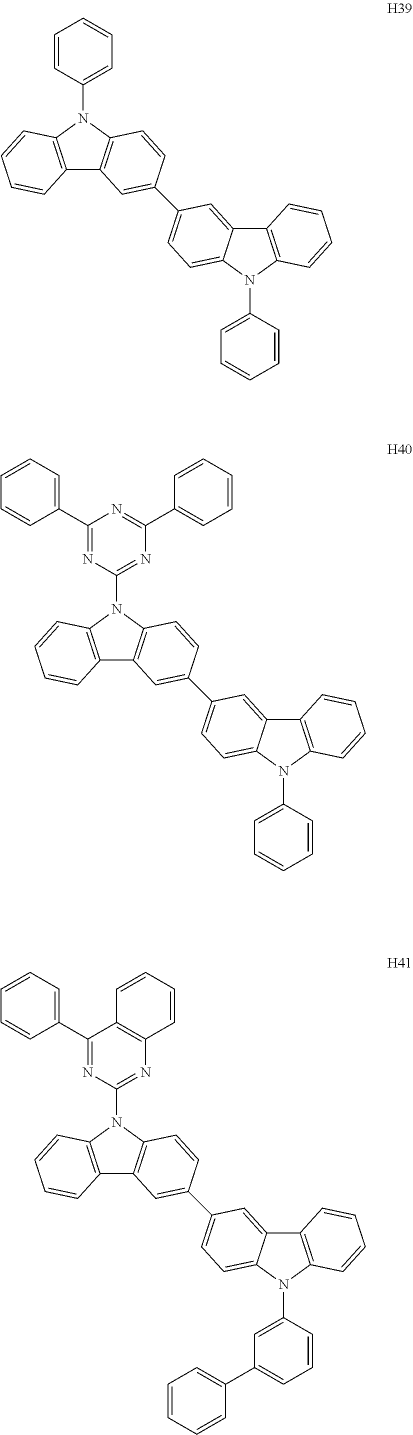 [SMILES: c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

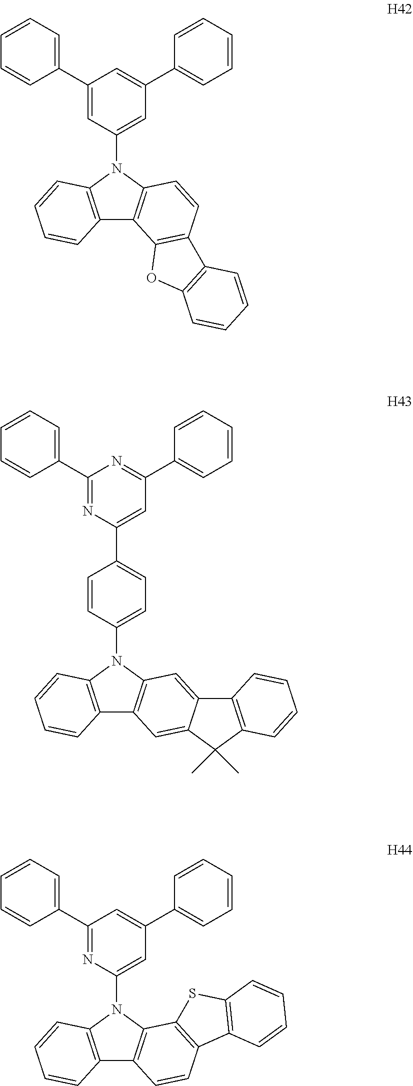 [SMILES: c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1]

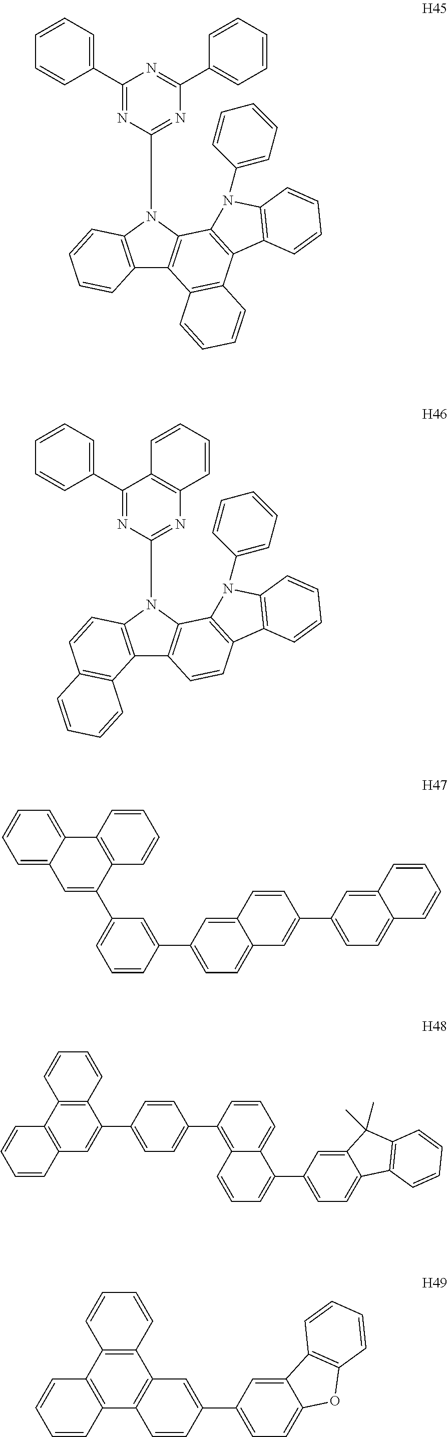

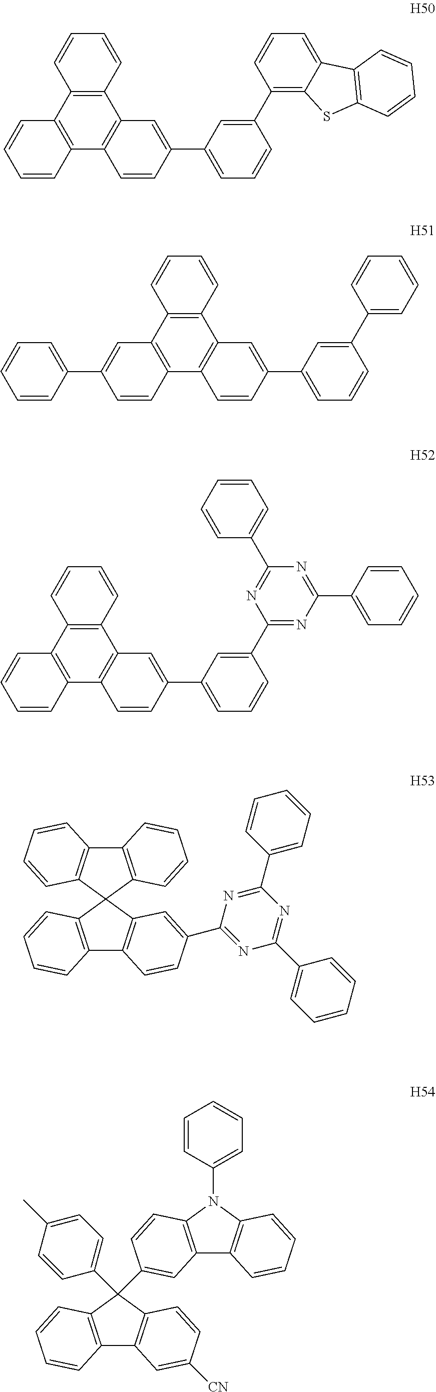

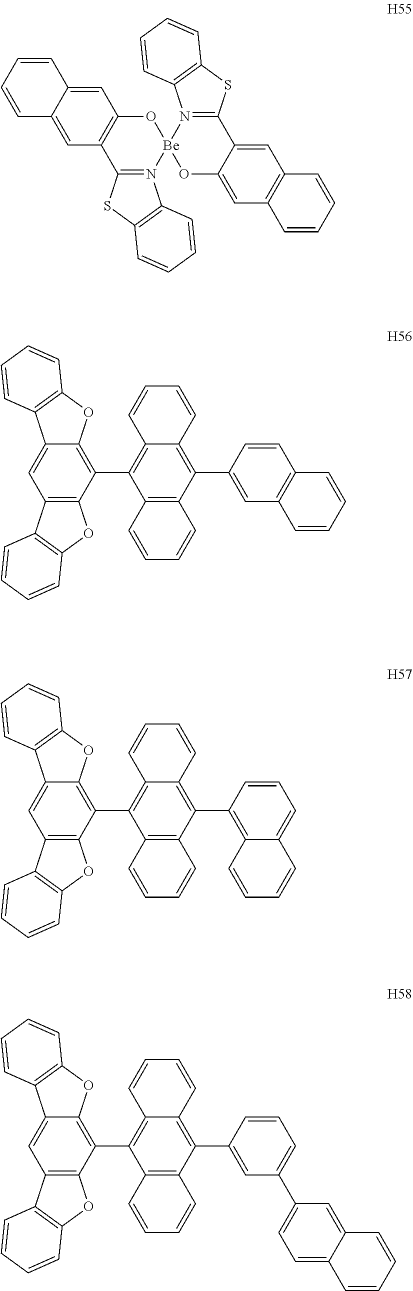

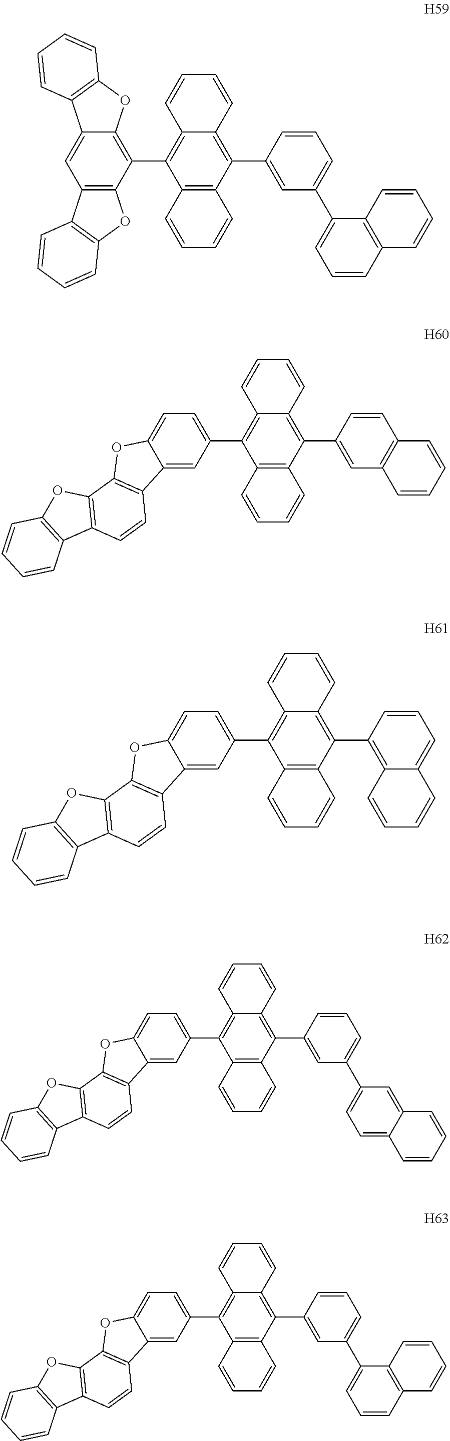 [SMILES: c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1]

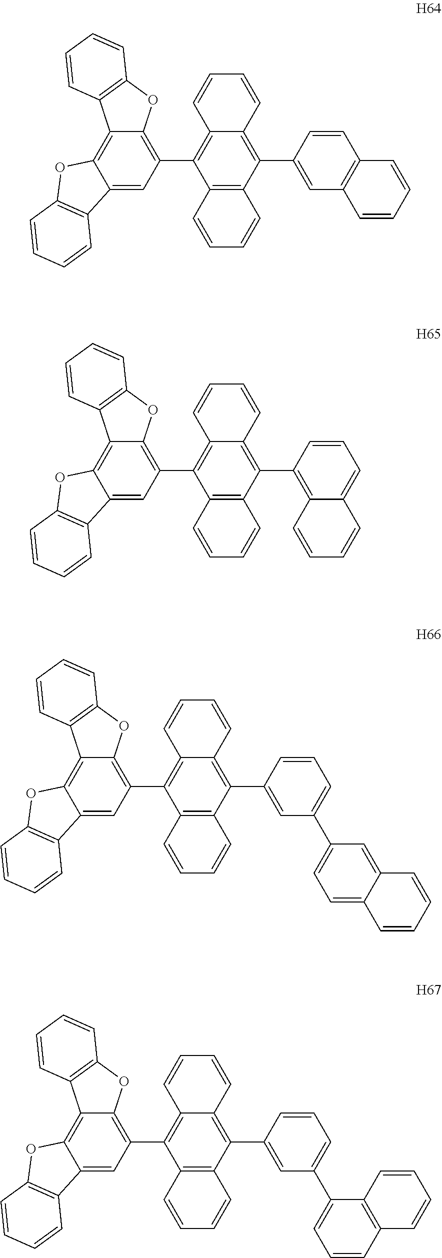 [SMILES: c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1]

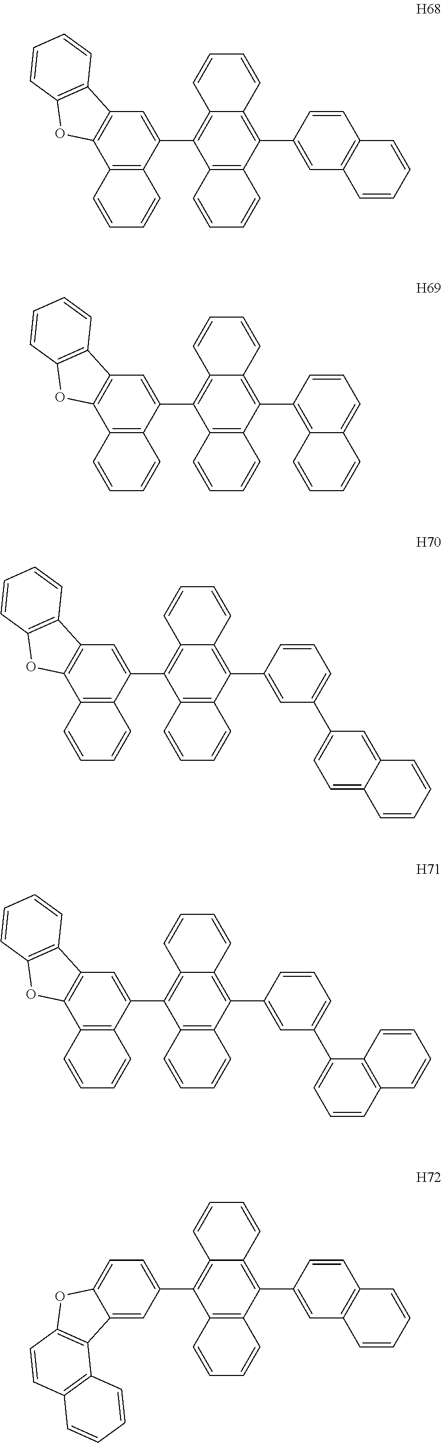 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

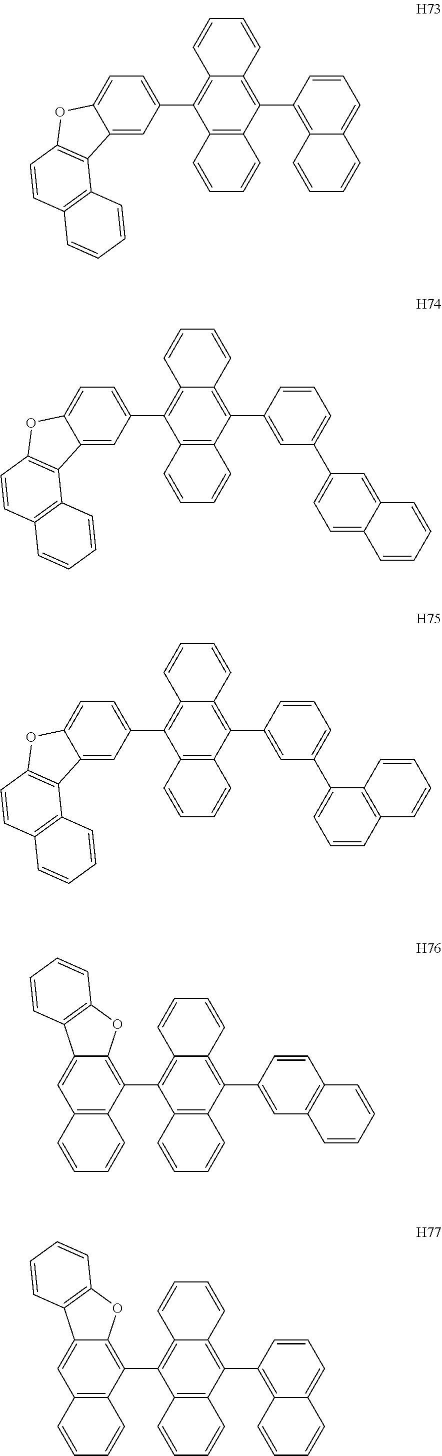 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1]

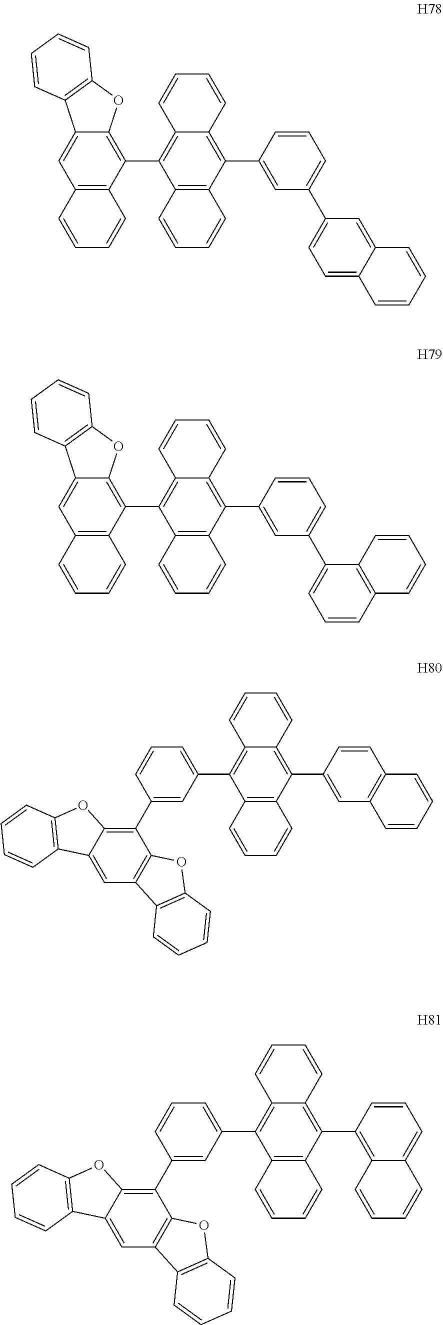 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

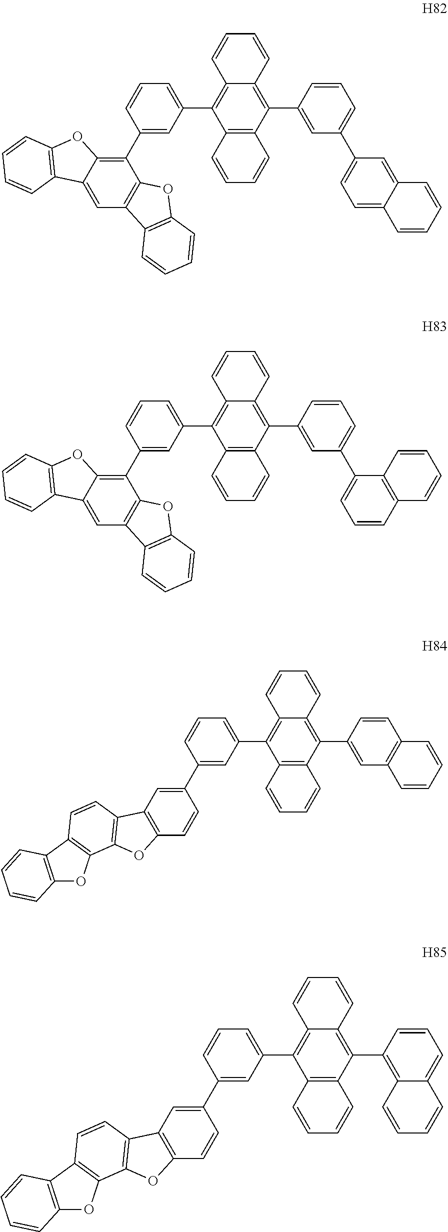 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1]

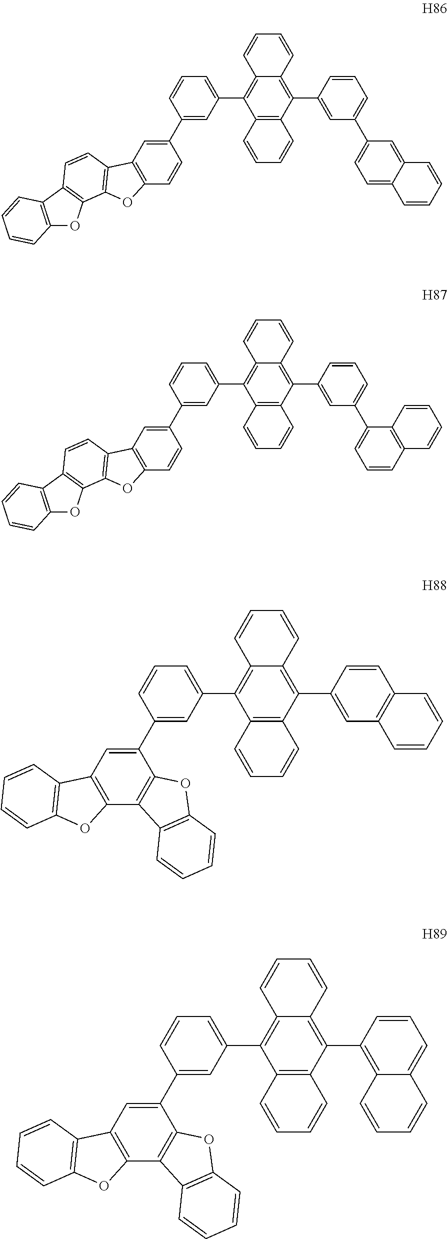 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

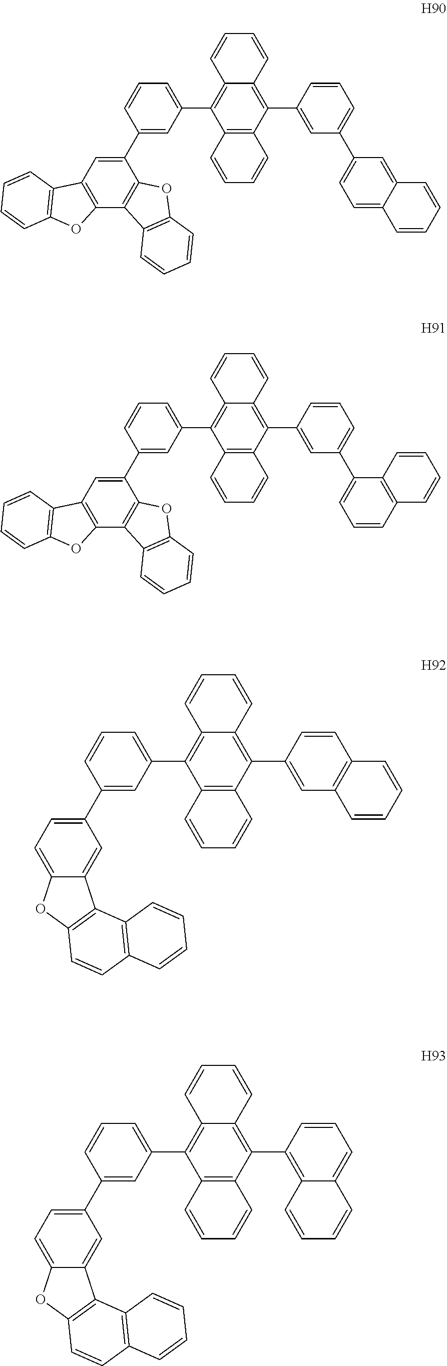 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

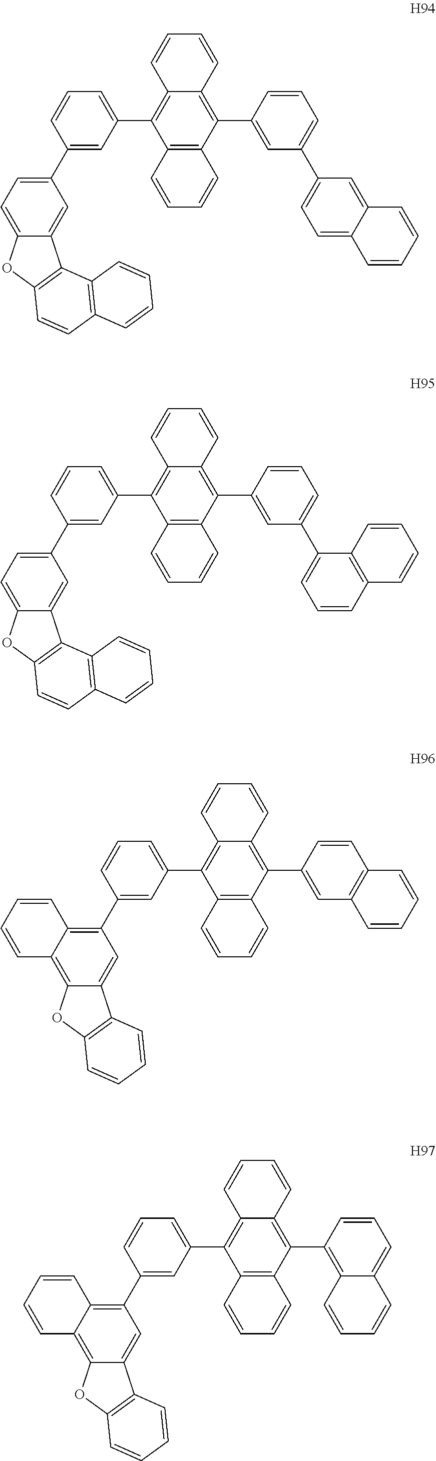 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

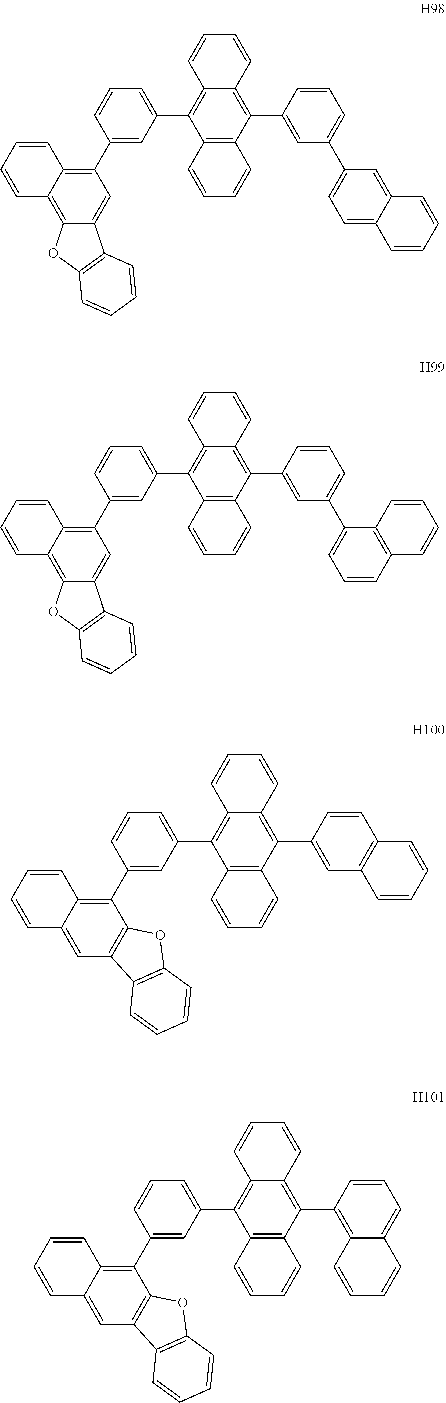 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

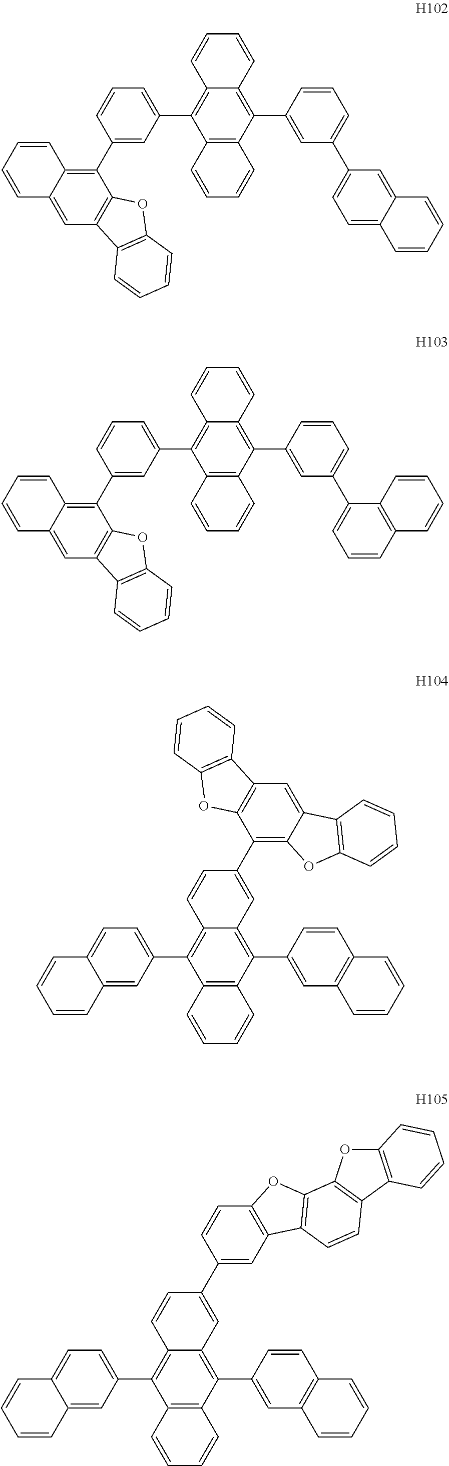 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

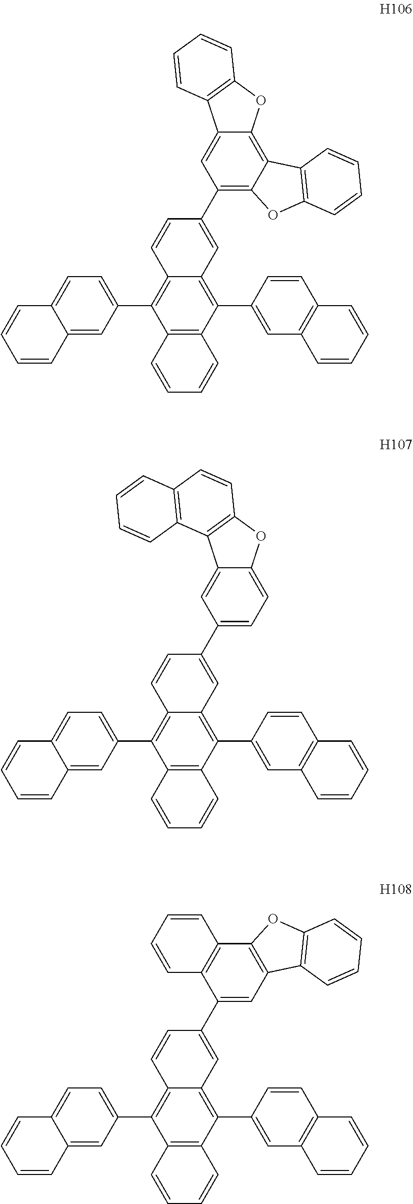 [SMILES: c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

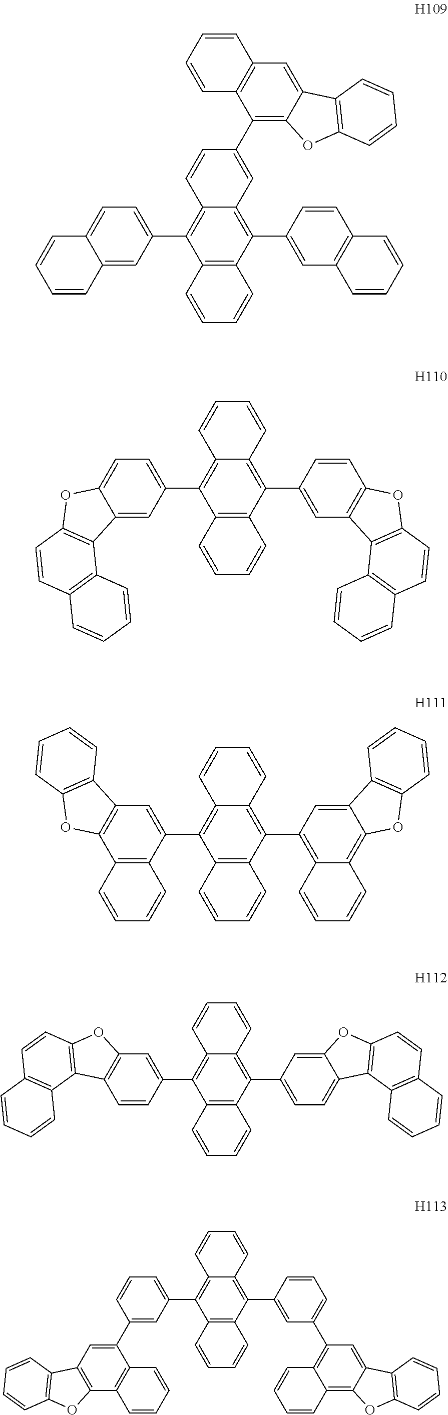 [SMILES: c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1]

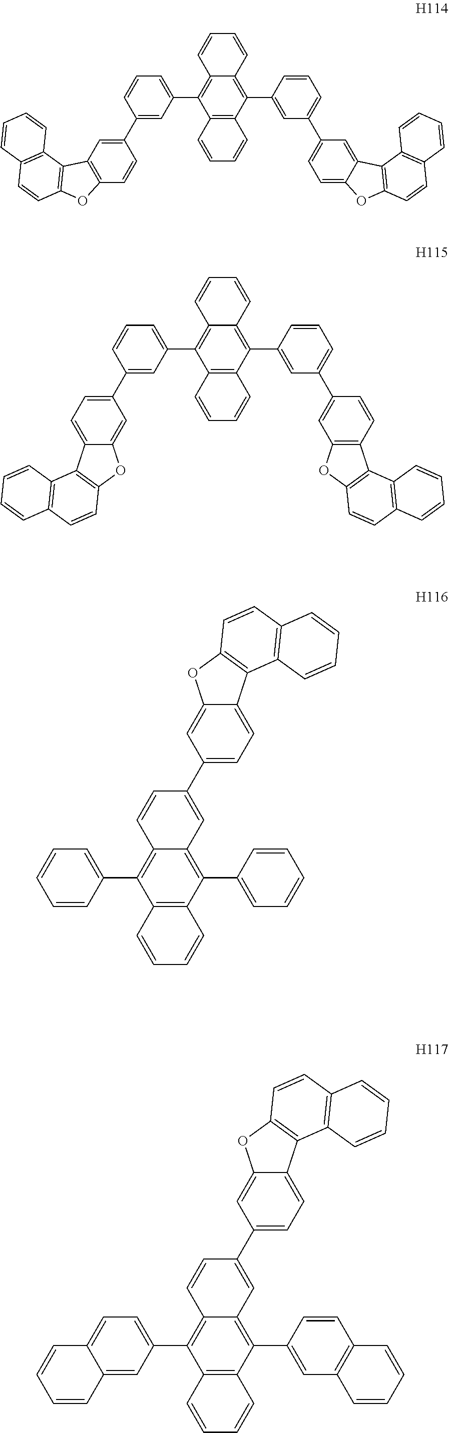 [SMILES: c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1]

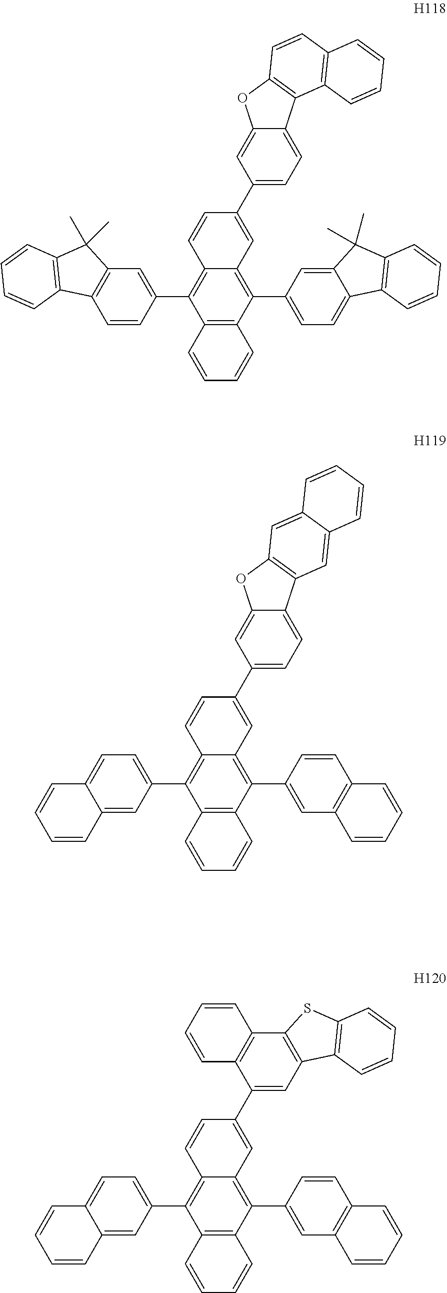 [SMILES: CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1]

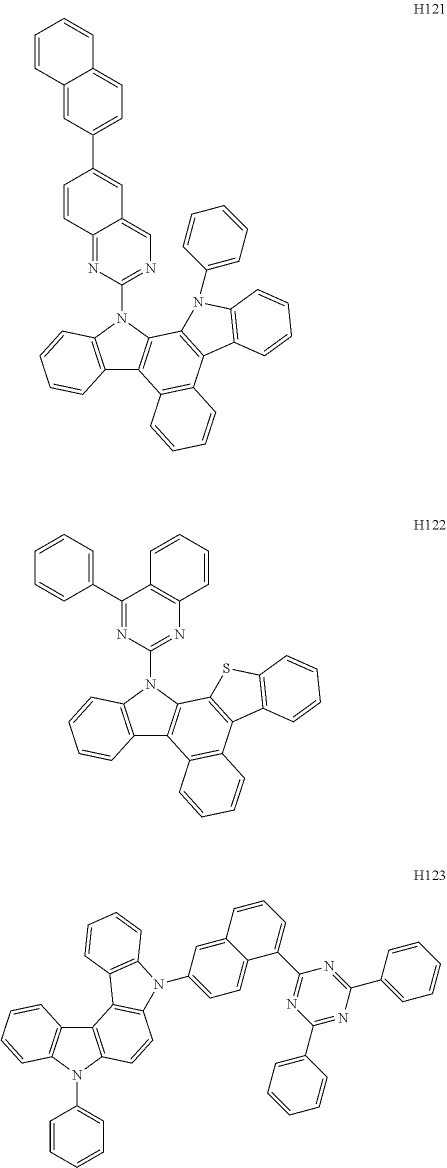 [SMILES: c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1]

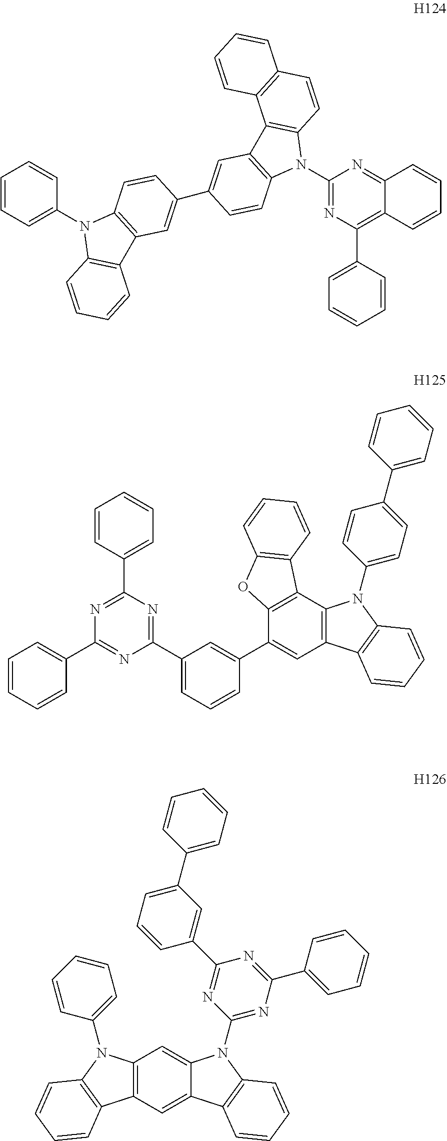 [SMILES: c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1]

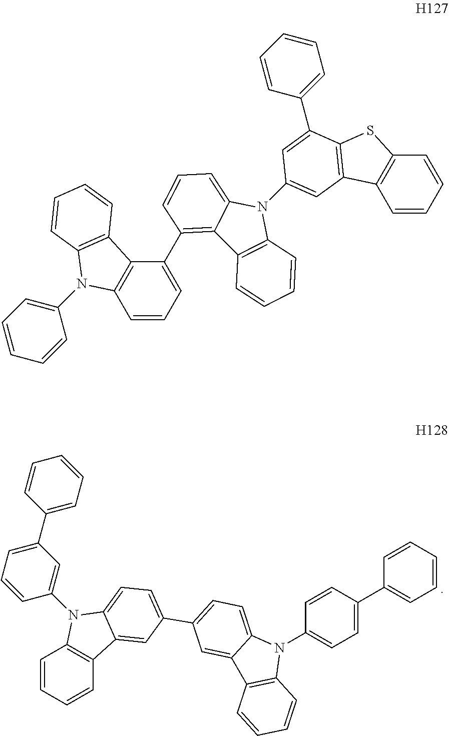 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

[Phosphorescent Dopant]

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

M(L₄₀₁)_{xc1}(L₄₀₂)_{xc2} Formula 401

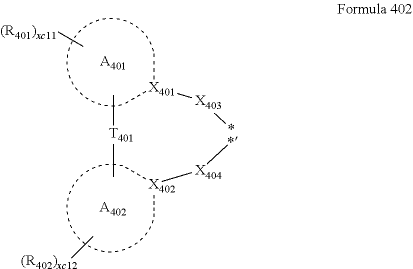

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁(s) may be identical to or different from each other,
L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)*′, *—N(Q₄₁₁)-*′, *—C(Q₄₁₁)(Q₄₁₂)-*′, *—C(Q₄₁₁)=C(Q₄₁₂)*′, *—C(Q₄₁₁)=*′, or *═C═*′,
X₄₀₃ and X₄₀₄ may each independently be a chemical bond (e.g., a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be as defined herein in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be as defined herein in connection with Q₁,
xc11 and xc12 may each independently be an integer from 0 to 10, and
* and *′ in Formula 402 may each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In an embodiment, when xc1 in Formula 401 is 2 or more, two ring A₄₀₁(s) in two or more of L₄₀₁(s) may optionally be linked to each other via T₄₀₂, which is a linking group, and two ring A₄₀₂(s) may optionally be linked to each other via T₄₀₃, which is a linking group (see Compounds PD1 to PD4 and PD7). T₄₀₂ and T₄₀₃ may each be as defined herein in connection with T₄₀₁.

L₄₀₂ in Formula 401 may be an organic ligand. For example, L₄₀₂ may include a halogen group, a diketone group (e.g., an acetylacetonate group), a carboxylic acid group (e.g., a picolinate group), —C(═O), an isonitrile group, a —CN group, a phosphorus group (e.g., a phosphine group, a phosphite group, etc.), or any combination thereof.

The phosphorescent dopant may include, for example, one of Compounds PD1 to PD39 or any combination thereof:

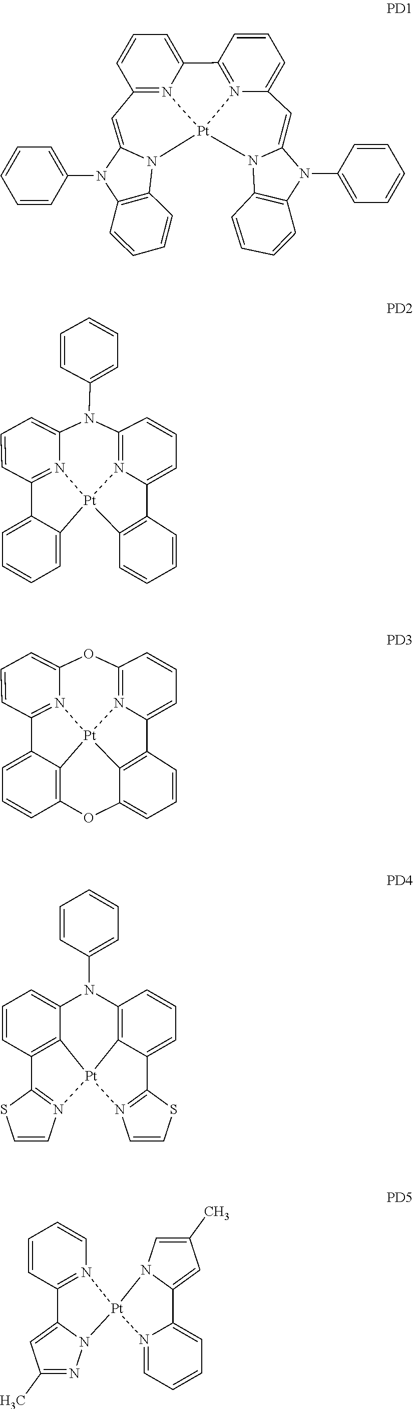

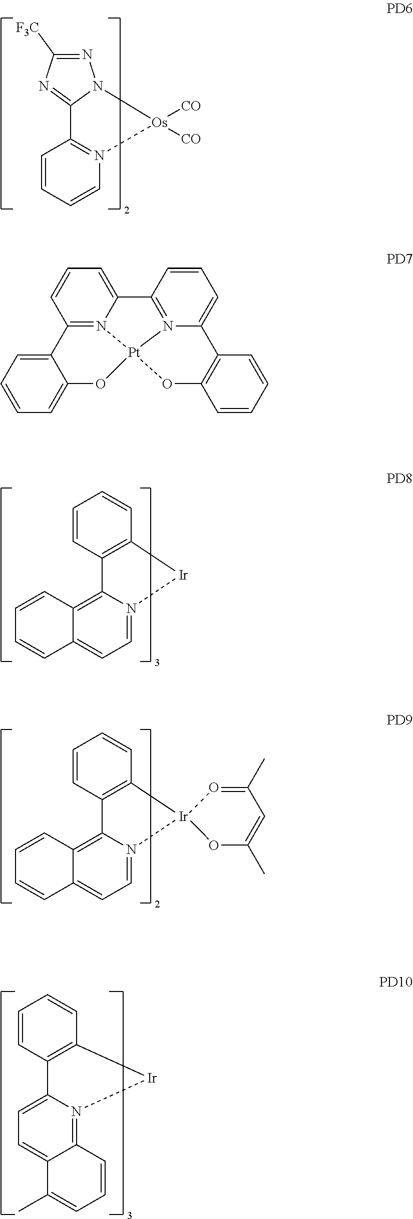

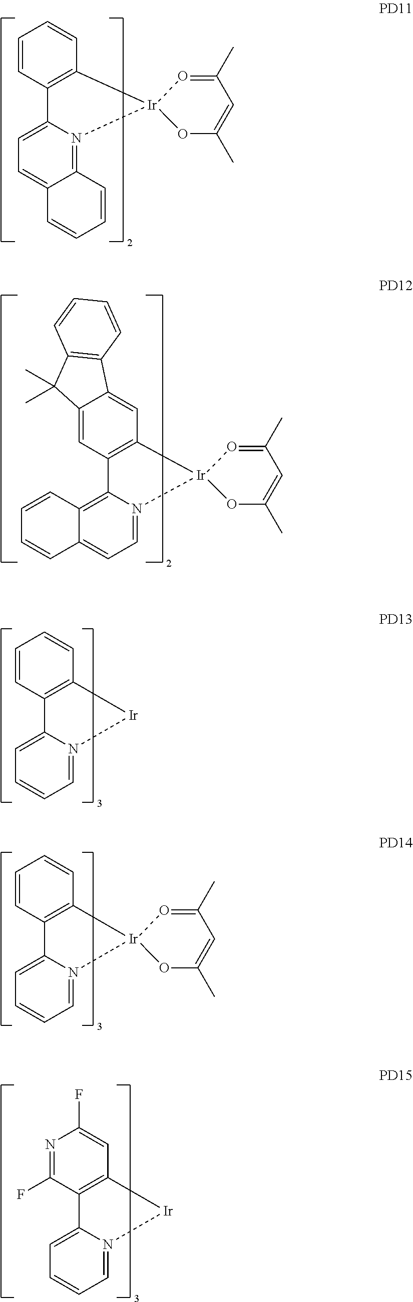

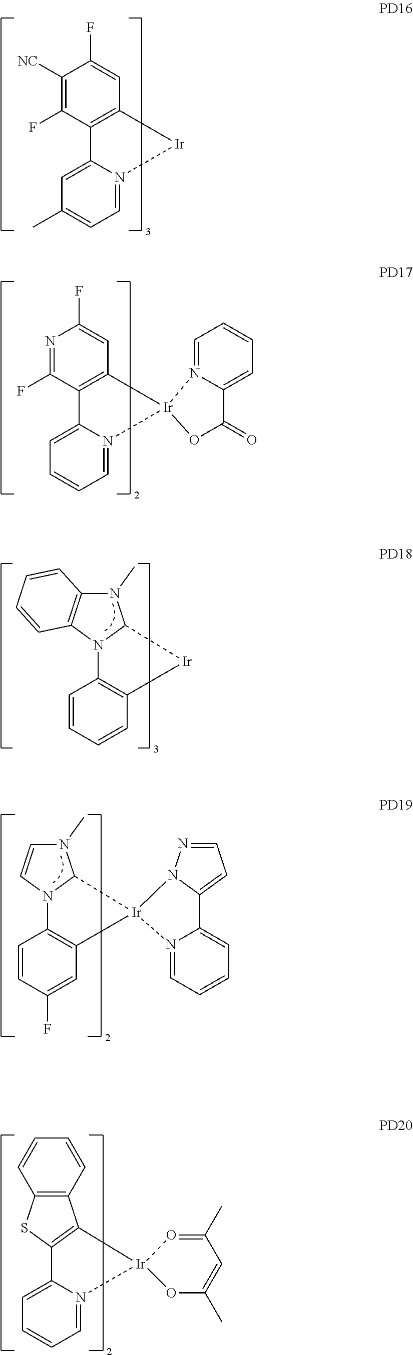

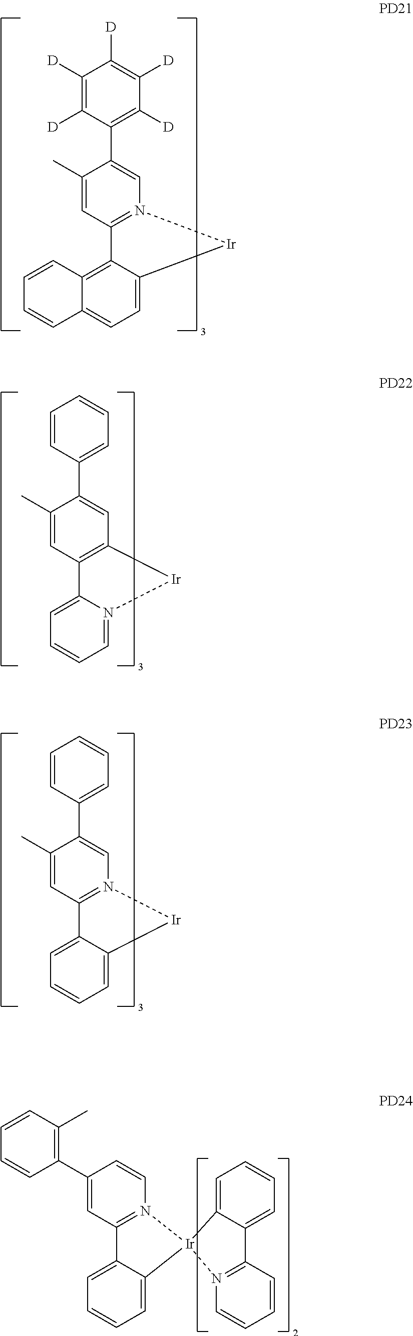

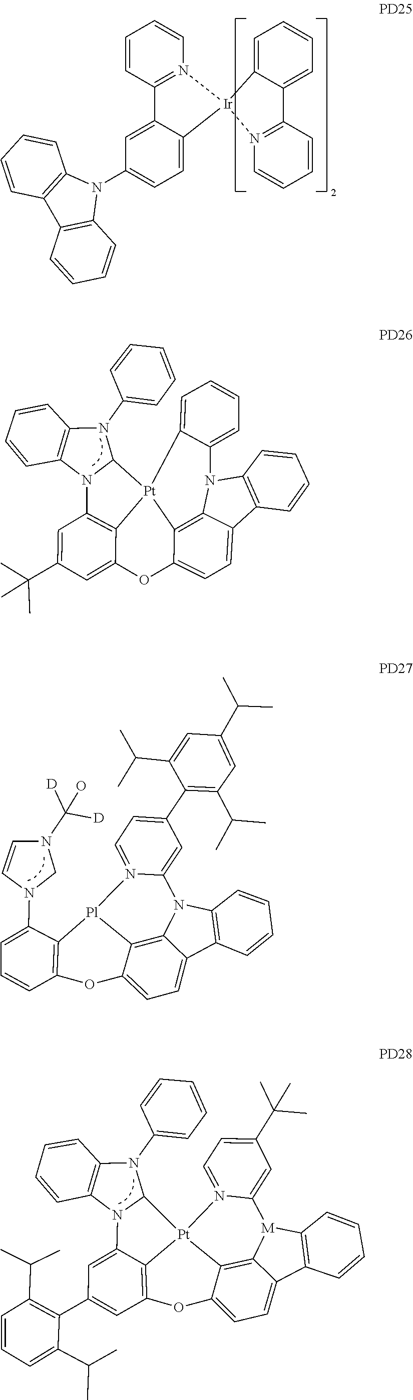

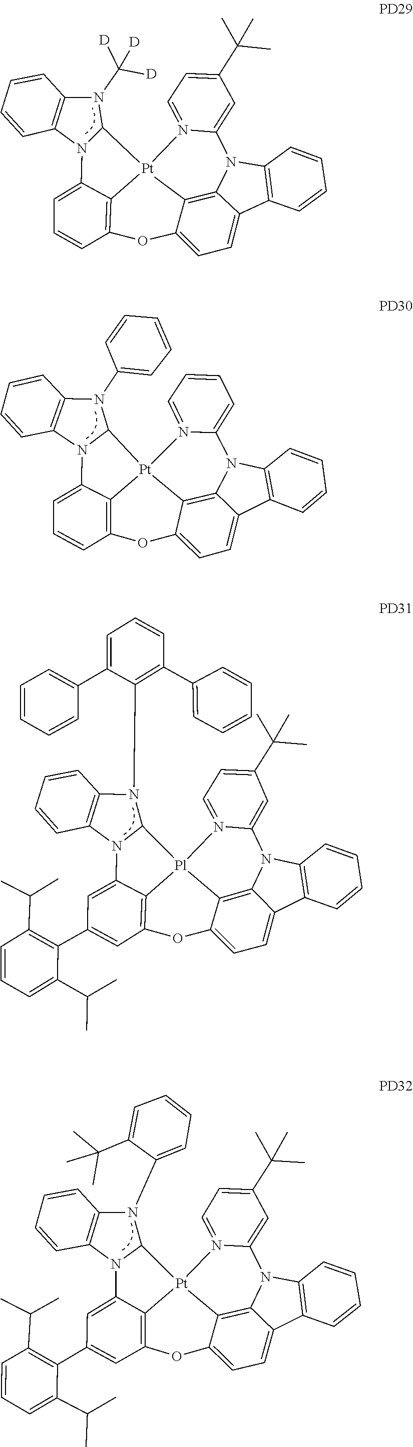

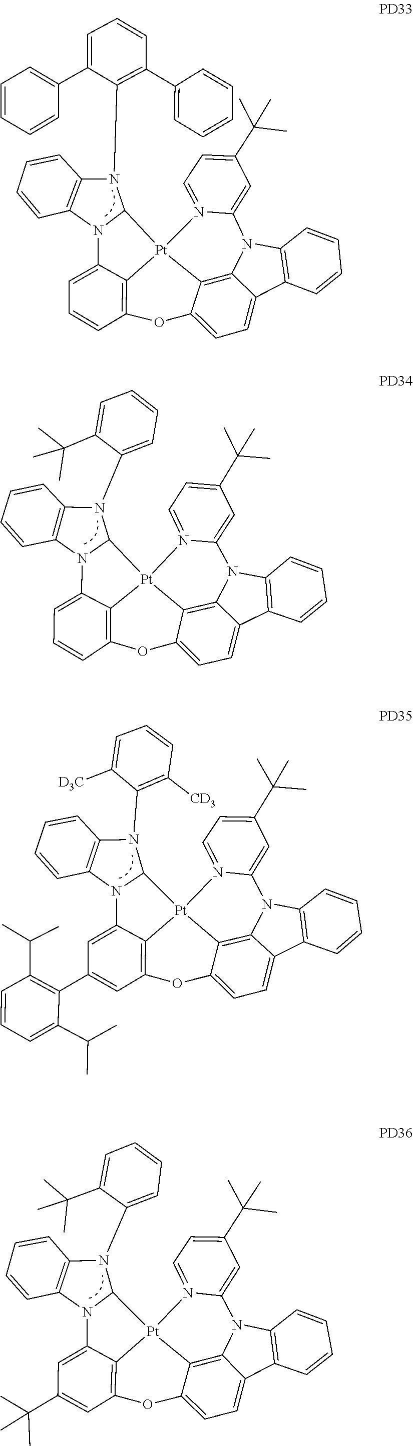

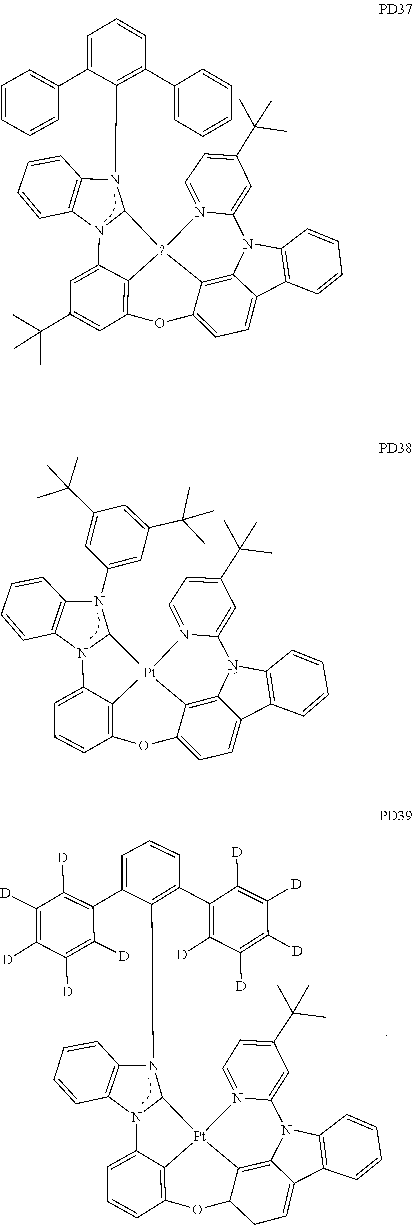

[Fluorescent Dopant]

The fluorescent dopant may include an amine group-containing compound, a styryl group-containing compound, or any combination thereof.

For example, the fluorescent dopant may include a compound represented by Formula 501:

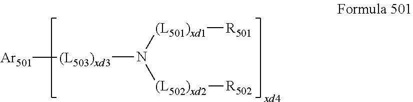

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (e.g., an anthracene group, a chrysene group, a pyrene group, etc.) in which three or more monocyclic groups are condensed with each other.

In one or more embodiments, xd4 in Formula 501 may be 2.

For example, the fluorescent dopant may include one of Compounds FD1 to FD37, DPVBi, DPAVBi, or any combination thereof:

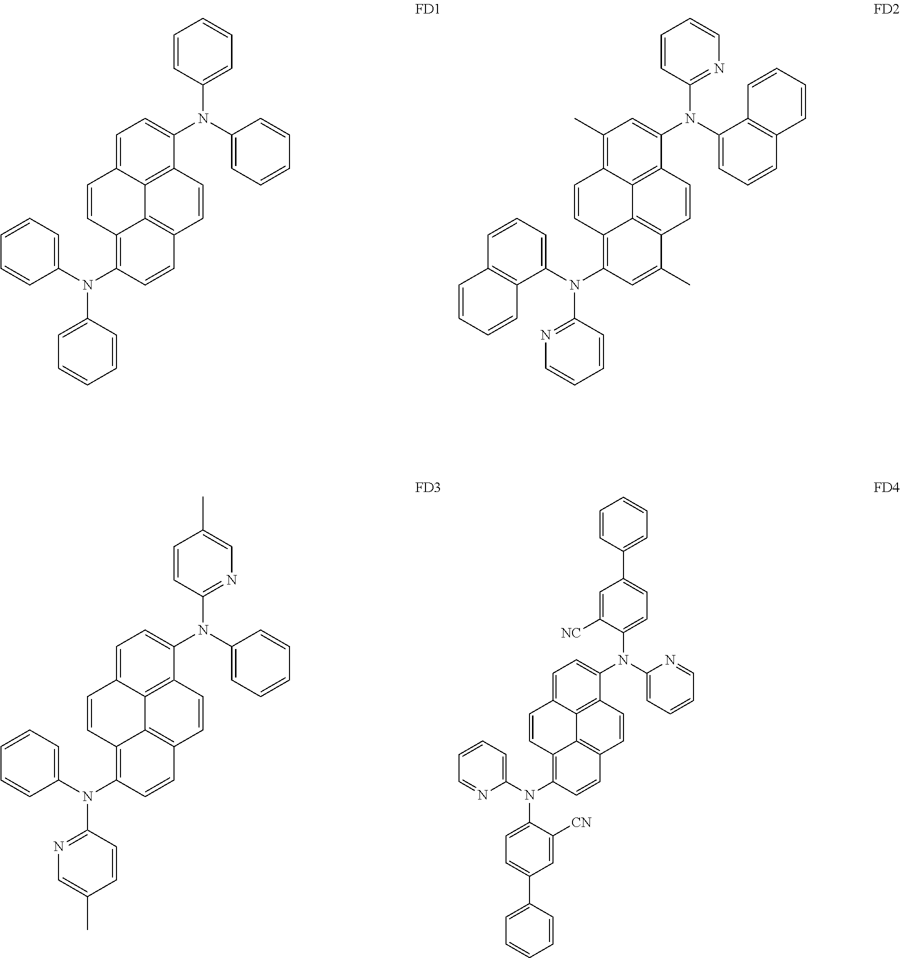 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1 ; N#Cc1cc(-c2ccccc2)ccc1N(c1ccccn1)c1ccc2ccc3c(N(c4ccccn4)c4ccc(-c5ccccc5)cc4C#N)ccc4ccc1c2c43]

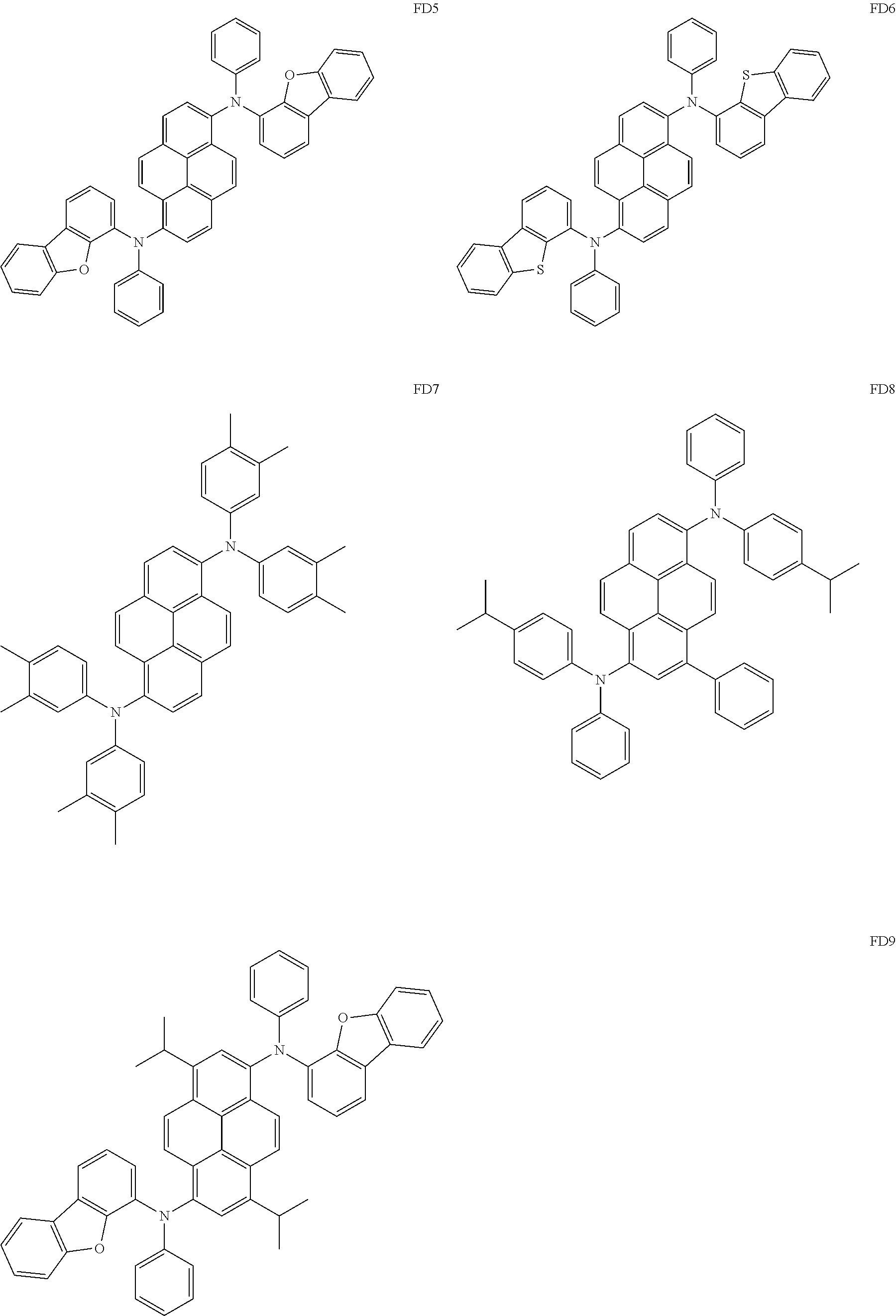

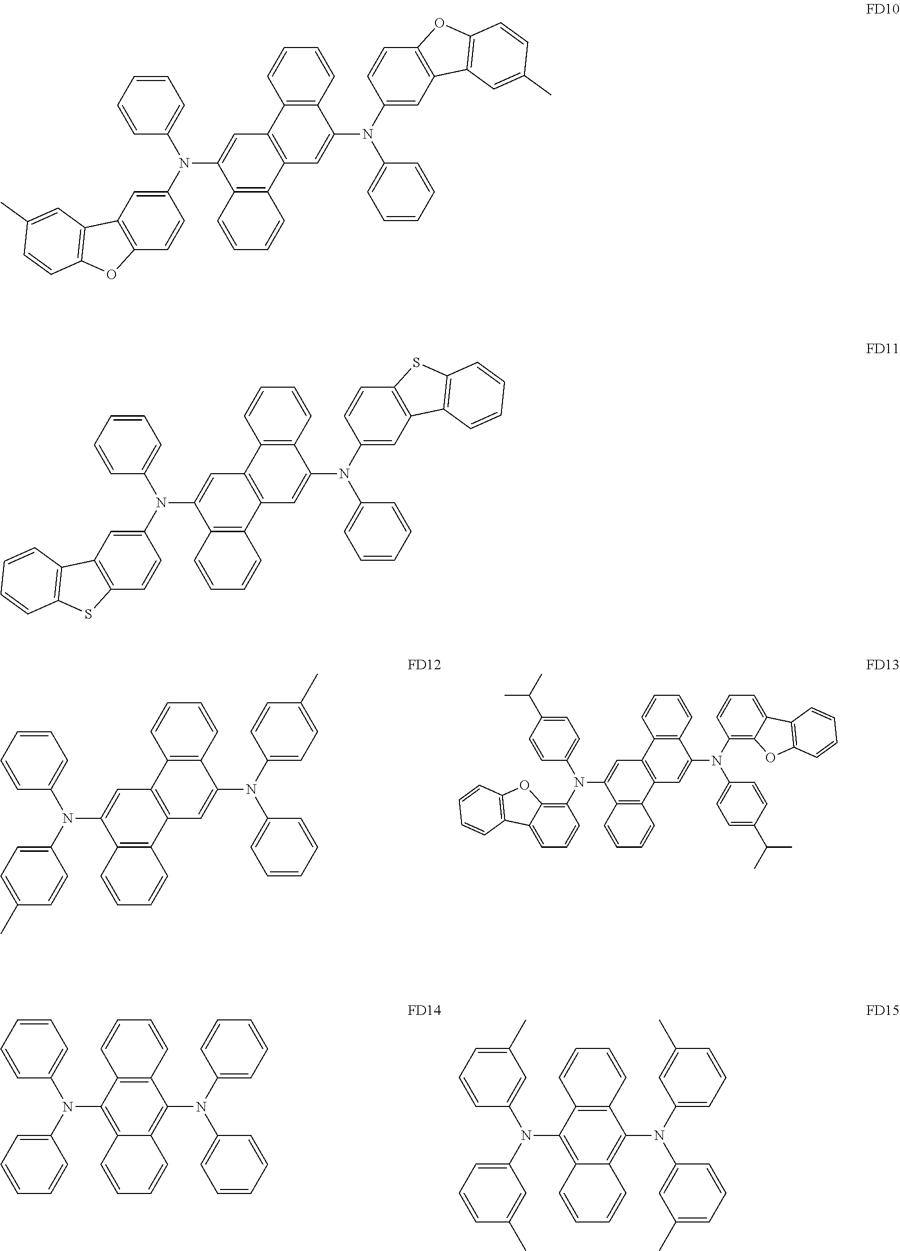

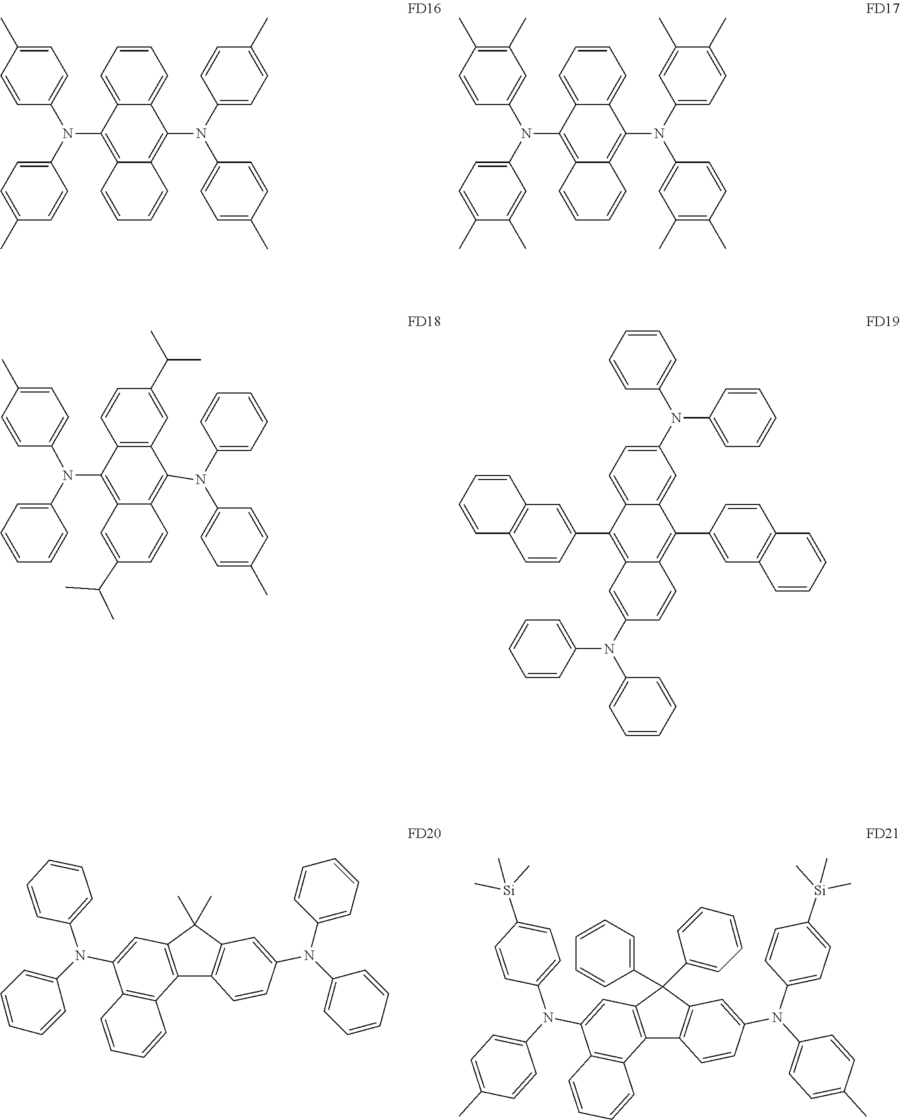

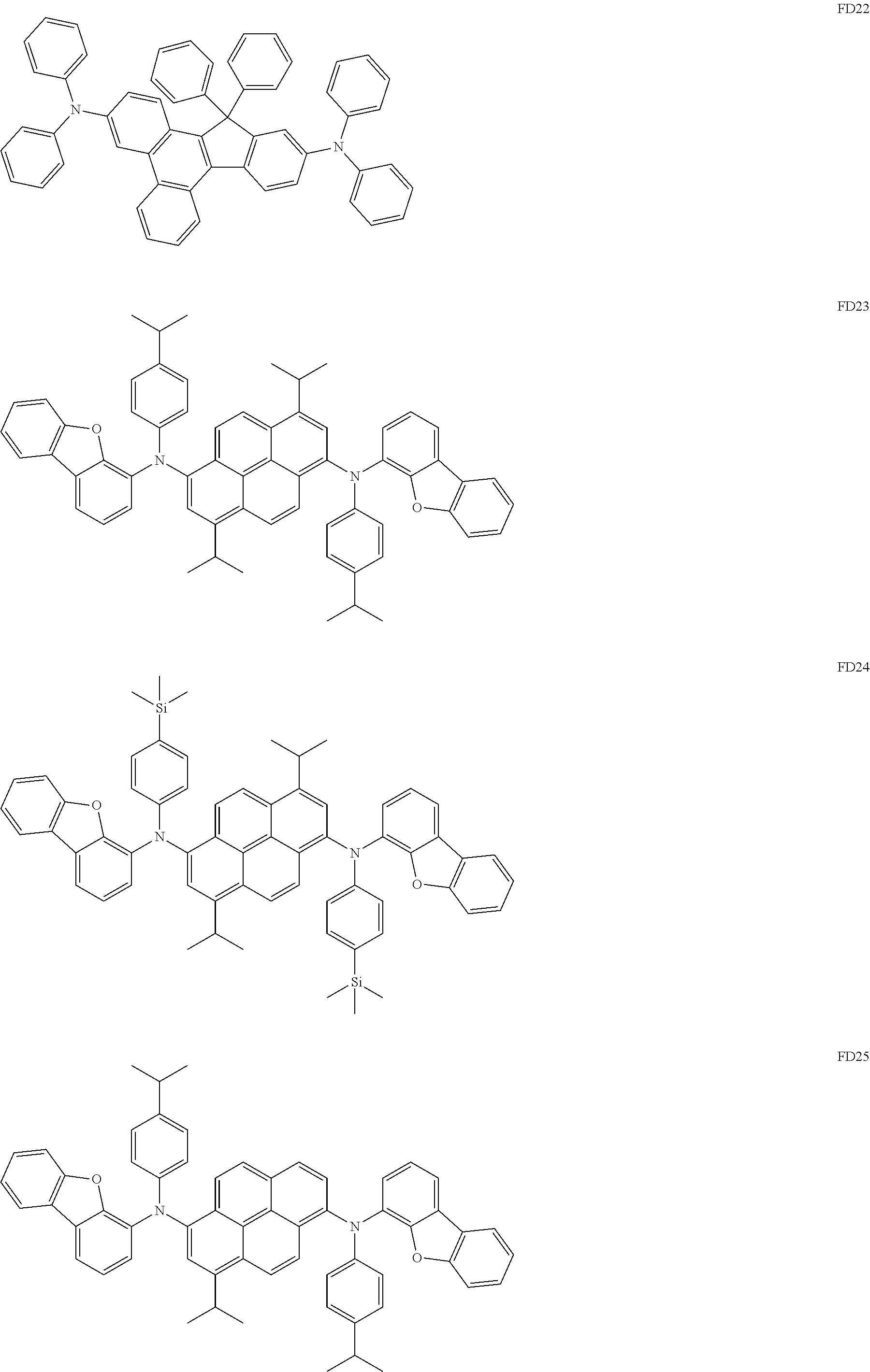 [SMILES: c1ccc(N(c2ccccc2)c2ccc3c(c2)C(c2ccccc2)(c2ccccc2)c2c-3c3ccccc3c3cc(N(c4ccccc4)c4ccccc4)ccc23)cc1 ; CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)cc(C(C)C)c5ccc2c3c54)c2cccc3c2oc2ccccc23)cc1]

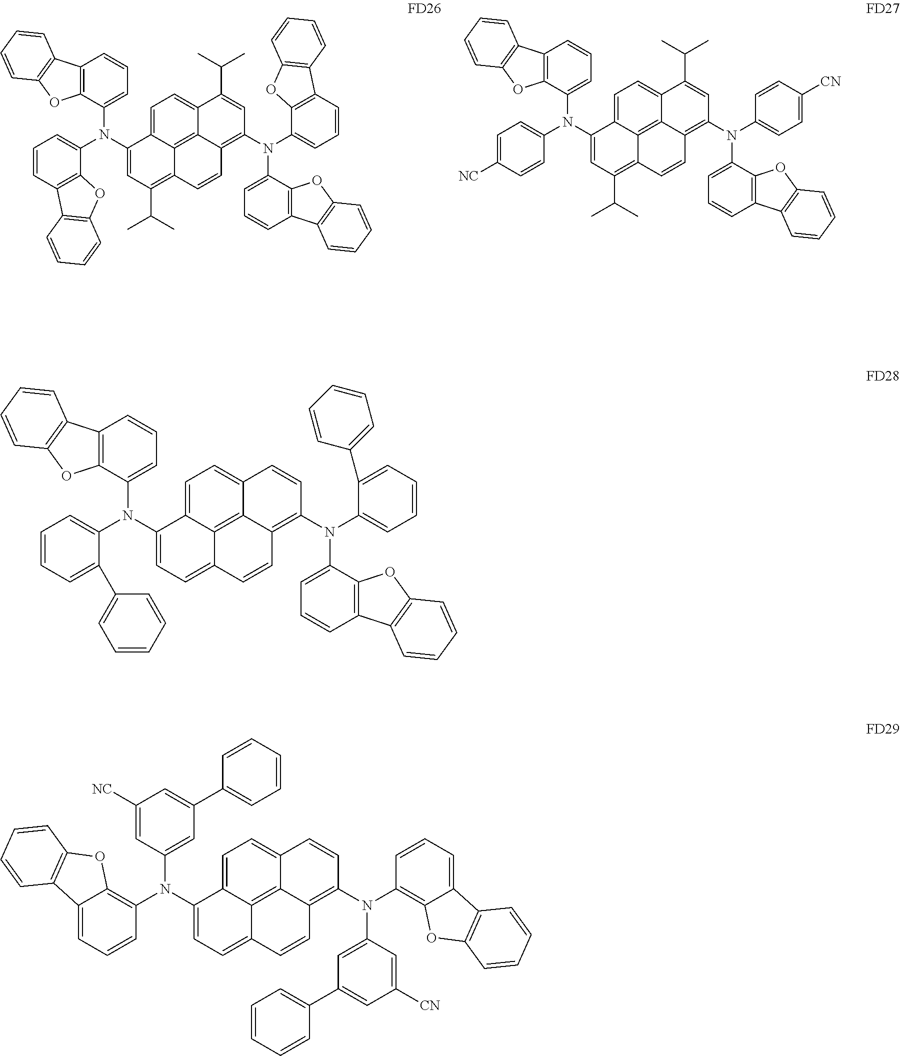 [SMILES: CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34 ; CC(C)c1cc(N(c2ccc(C#N)cc2)c2cccc3c2oc2ccccc23)c2ccc3c(C(C)C)cc(N(c4ccc(C#N)cc4)c4cccc5c4oc4ccccc45)c4ccc1c2c34 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; N#Cc1cc(-c2ccccc2)cc(N(c2ccc3ccc4c(N(c5cc(C#N)cc(-c6ccccc6)c5)c5cccc6c5oc5ccccc56)ccc5ccc2c3c54)c2cccc3c2oc2ccccc23)c1]

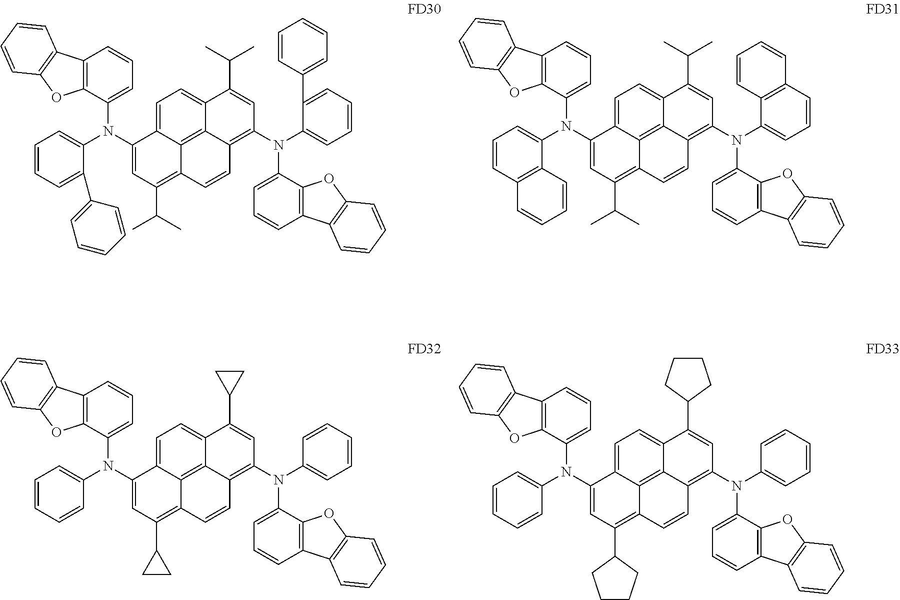 [SMILES: CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34 ; CC(C)c1cc(N(c2cccc3ccccc23)c2cccc3c2oc2ccccc23)c2ccc3c(C(C)C)cc(N(c4cccc5ccccc45)c4cccc5c4oc4ccccc45)c4ccc1c2c34 ; c1ccc(N(c2cc(C3CC3)c3ccc4c(N(c5ccccc5)c5cccc6c5oc5ccccc56)cc(C5CC5)c5ccc2c3c54)c2cccc3c2oc2ccccc23)cc1 ; c1ccc(N(c2cc(C3CCCC3)c3ccc4c(N(c5ccccc5)c5cccc6c5oc5ccccc56)cc(C5CCCC5)c5ccc2c3c54)c2cccc3c2oc2ccccc23)cc1]

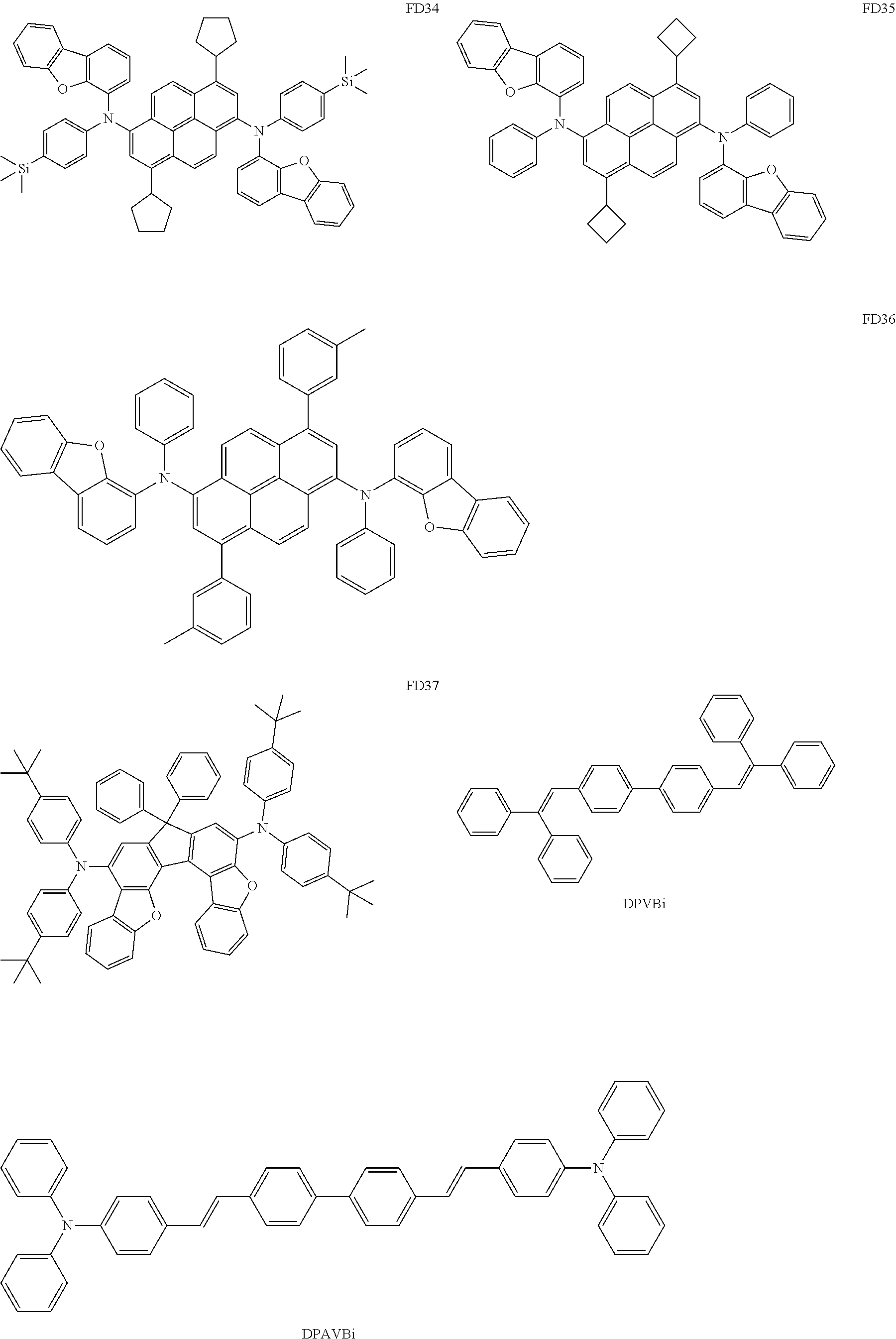

[Delayed Fluorescence Material]

The emission layer 130 may include a delayed fluorescence material.

The delayed fluorescence material as used herein may be selected from compounds capable of emitting delayed fluorescent light based on a delayed fluorescence emission mechanism.

The delayed fluorescence material included in the emission layer 130 may serve as a host or a dopant depending on the type of other materials included in the emission layer 130.

In an embodiment, a difference between a triplet energy level (eV) of the delayed fluorescence material and a singlet energy level (eV) of the delayed fluorescence material may be 0 eV or more and 0.5 eV or less. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (e.g., a π electron-rich C₃-C₆₀ cyclic group, such as a carbazole group, etc.) and at least one electron acceptor (e.g., a sulfoxide group, a cyano group, a π electron-deficient nitrogen-containing C₁-C₆₀ cyclic group, etc.), and ii) a material including a C₈-C₆₀ polycyclic group in which two or more cyclic groups are condensed with each other while sharing boron (B).

Examples of the delayed fluorescence material may include at least one of Compounds DF1 to DF14:

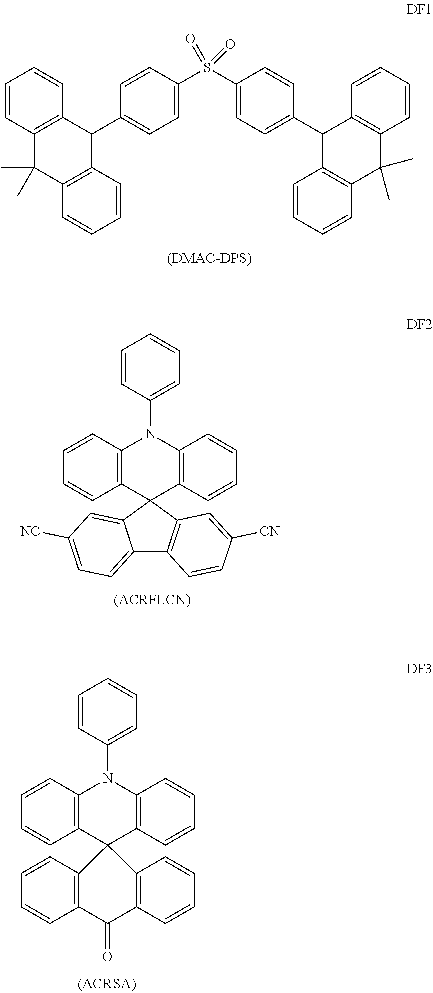

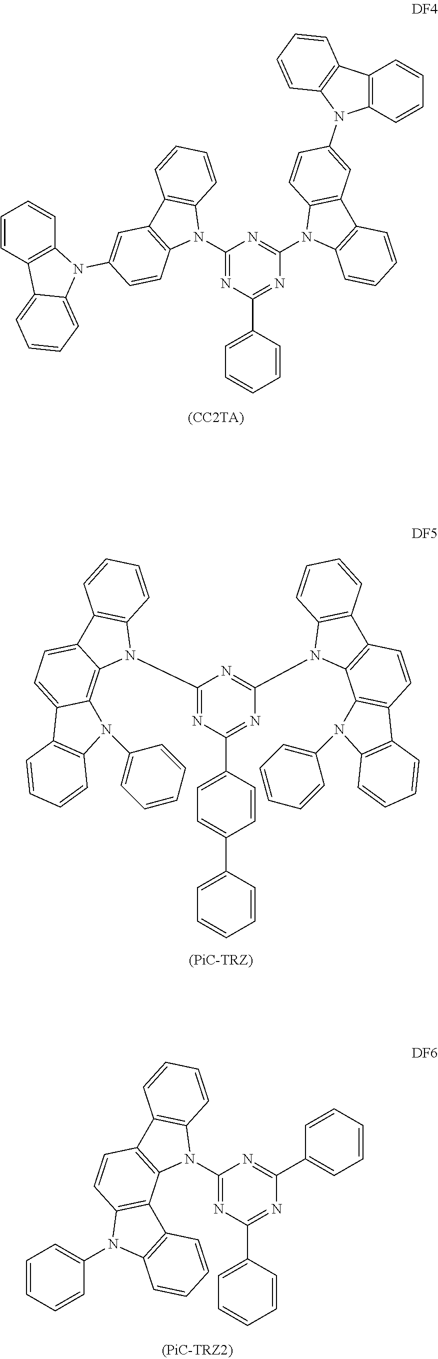 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

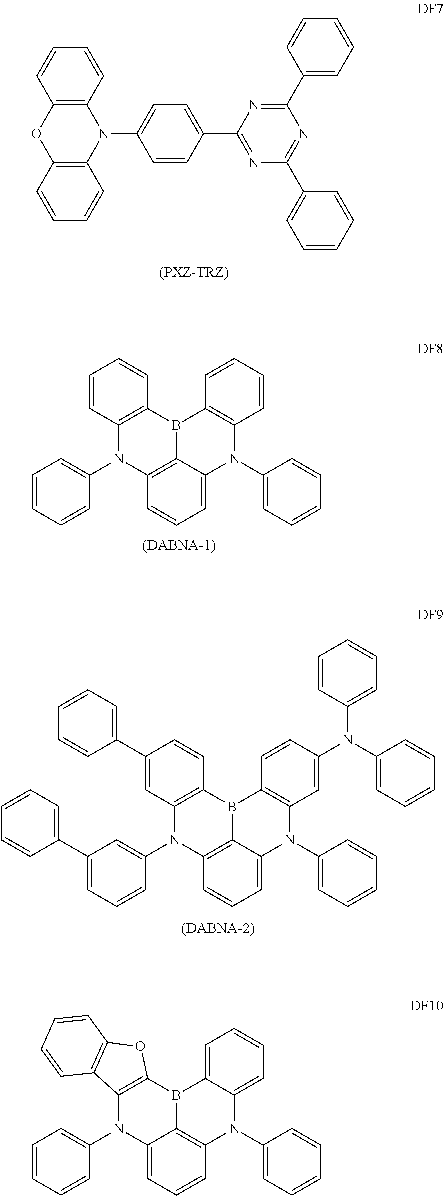

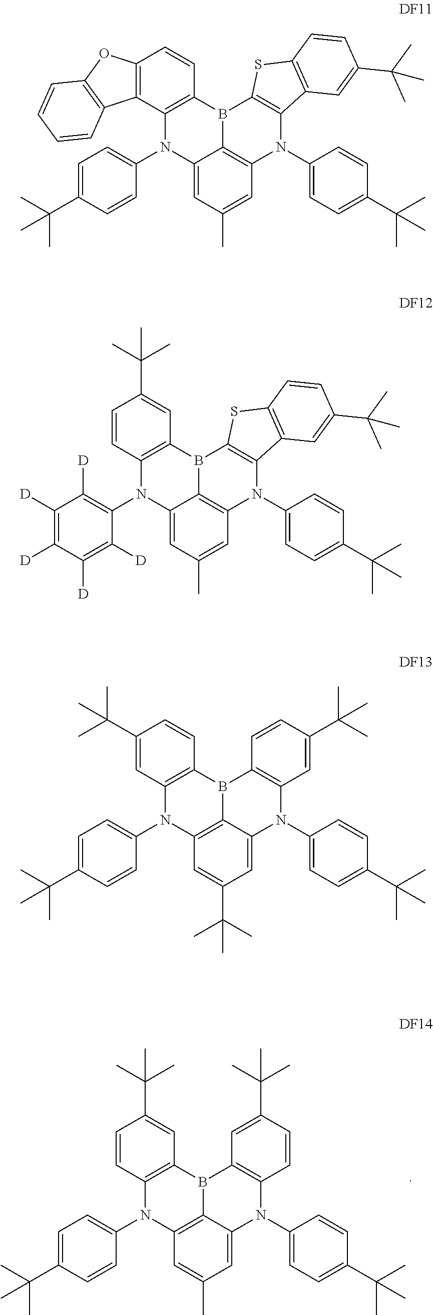

[Quantum Dot]

The emission layer 130 may include a quantum dot.

The term “quantum dot” as used herein refers to a crystal of a semiconductor compound, and may include any material capable of emitting light of various emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. In an embodiment, the Group III-V semiconductor compound may further include a Group II element. Examples of the Group III-V semiconductor compound further including a Group II element may include InZnP, InGaZnP, InAlZnP, and the like.

Examples of the Group III-VI semiconductor compound may include: a binary compound, such as GaS, GaSe, Ga₂Se₃, GaTe, InS, InSe, In₂S₃, In₂Se₃, or InTe; a ternary compound, such as InGaS₃ or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, or AgAlO₂; a quaternary compound, such as AgInGaS or AgInGaS₂; or any combination thereof.

Examples of the Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

The Group IV element or compound may include: a single element, such as Si or Ge; a binary compound, such as SiC or SiGe; or any combination thereof.

Each element included in a multi-element compound, such as the binary compound, the ternary compound, and the quaternary compound, may be present at a uniform concentration or non-uniform concentration in a particle.

In an embodiment, the quantum dot may have a single structure, in which the concentration of each element in the quantum dot is uniform, or a core-shell dual structure. For example, a material included in the core and a material included in the shell may be different from each other.

The shell of the quantum dot may serve as a protective layer that prevents chemical degeneration of the core to maintain semiconductor characteristics, and/or as a charging layer that imparts electrophoretic characteristics to the quantum dot. The shell may be a single layer or a multi-layer. An interface between the core and the shell may have a concentration gradient in which the concentration of an element existing in the shell decreases toward the center of the core.

Examples of the shell of the quantum dot may include an oxide of metal, metalloid, or non-metal, a semiconductor compound, or any combination thereof. Examples of the oxide of metal, metalloid, or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity or color reproducibility may be improved. In addition, since light emitted through the quantum dot is emitted in all directions, an optical viewing angle may be improved.

In addition, the quantum dot may be in the form of a spherical particle, a pyramidal particle, a multi-arm particle, a cubic nanoparticle, a nanotube particle, a nanowire particle, a nanofiber particle, or a nanoplate particle.

Since the energy band gap may be adjusted by controlling the size of the quantum dot, light having various wavelength bands may be obtained from an emission layer including the quantum dot. Accordingly, by using quantum dots of different sizes, a light-emitting device that emits light of various wavelengths may be implemented. In detail, the size of the quantum dot may be selected to emit red, green and/or blue light. In addition, the size of the quantum dot may be configured to emit white light by combining light of various colors.

[Photoactive Layer 135]

The opto-electronic device 30 may include the photoactive layer 135 arranged on the hole transport region 120. The photoactive layer 135 may include a first layer 131 adjacent to the hole transport region 120 and a second layer 132 adjacent to the electron transport region 140.

The emission layer 130 may emit light to the outside of the electronic apparatus. The light may be incident on an external object. For example, the object may be a finger of a user of the electronic apparatus. Light reflected by the object may be incident on the electronic apparatus.

The photoactive layer 135 may absorb the light incident on the electronic apparatus to form excitons. The excitons may generate holes and electrons. That is, the photoactive layer 135 may absorb light to generate an electrical signal. In detail, the first compound included in the photoactive layer 135 may serve as a donor for supplying electrons, and the second compound included in the photoactive layer 135 may serve as an acceptor for receiving electrons. That is, the opto-electronic device 30 may detect light energy and convert the detected light energy into an electrical signal. Accordingly, the opto-electronic device 30 may recognize an object that has come into contact with (or approached) the electronic apparatus. Accordingly, the opto-electronic device 30 including the photoactive layer 135 may serve as an optical sensor (e.g., a fingerprint recognition sensor). In some embodiments, the opto-electronic device 30 may be used in combination with the light-emitting device 10, for example as a touchscreen display device.

[Electron Transport Region 140]

The electron transport region 140 may have i) a single-layered structure consisting of a single layer consisting of a single material, ii) a single-layered structure consisting of a single layer including a plurality of different materials, or iii) a multi-layered structure including a plurality of layers including different materials.

The electron transport region 140 may include a buffer layer, a hole-blocking layer, an electron control layer, an electron transport layer, an electron injection layer, or any combination thereof.

For example, the electron transport region 140 may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the layers of each structure being sequentially stacked from the emission layer 130.

The electron transport region 140 (e.g., the buffer layer, the hole-blocking layer, the electron control layer, or the electron transport layer in the electron transport region 140) may include a metal-free compound including at least one π electron-deficient nitrogen-containing C₁-C₆₀ cyclic group.

For example, the electron transport region 140 may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be as defined herein in connection with Q₁,
xe21 may be 1, 2, 3, 4, or 5, and

at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be an anthracene group unsubstituted or substituted with at least one R₁₀ₐ.

In one or more embodiments, the electron transport region 140 may include a compound represented by Formula 601-1:

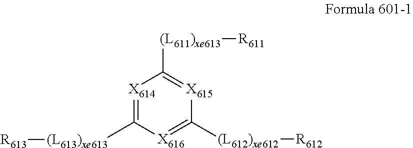

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each be as defined herein in connection with L₆₀₁,
xe611 to xe613 may each be as defined herein in connection with xe1,
R₆₁₁ to R₆₁₃ may each be as defined herein in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region 140 may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

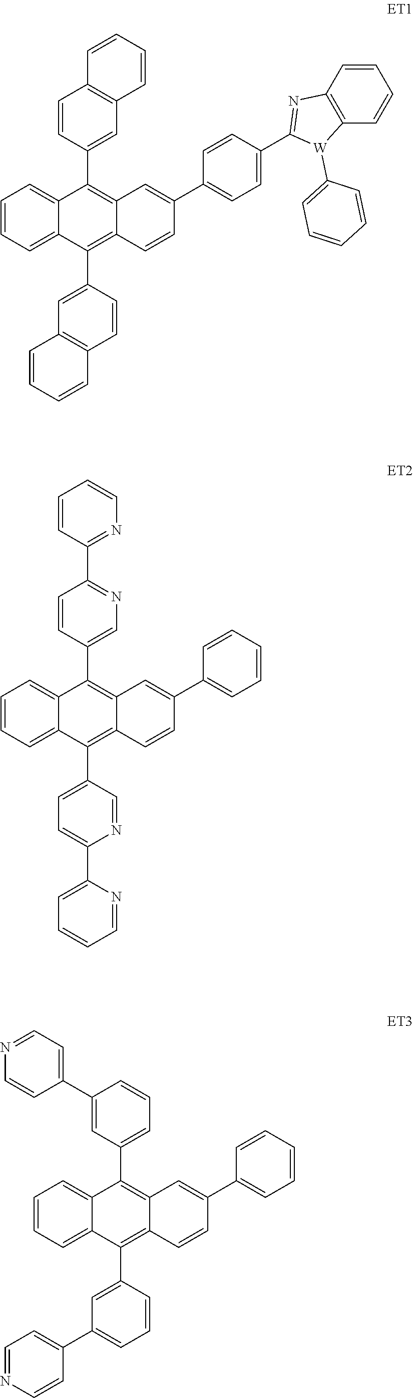

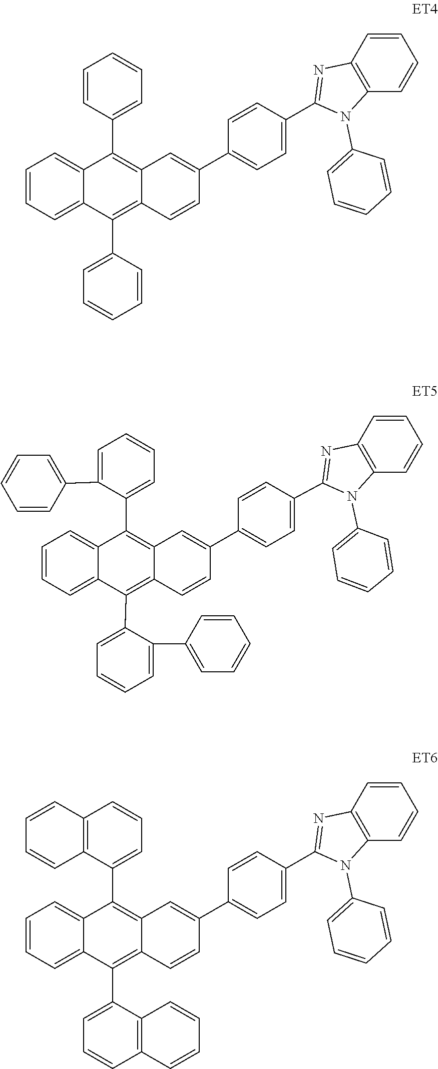 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

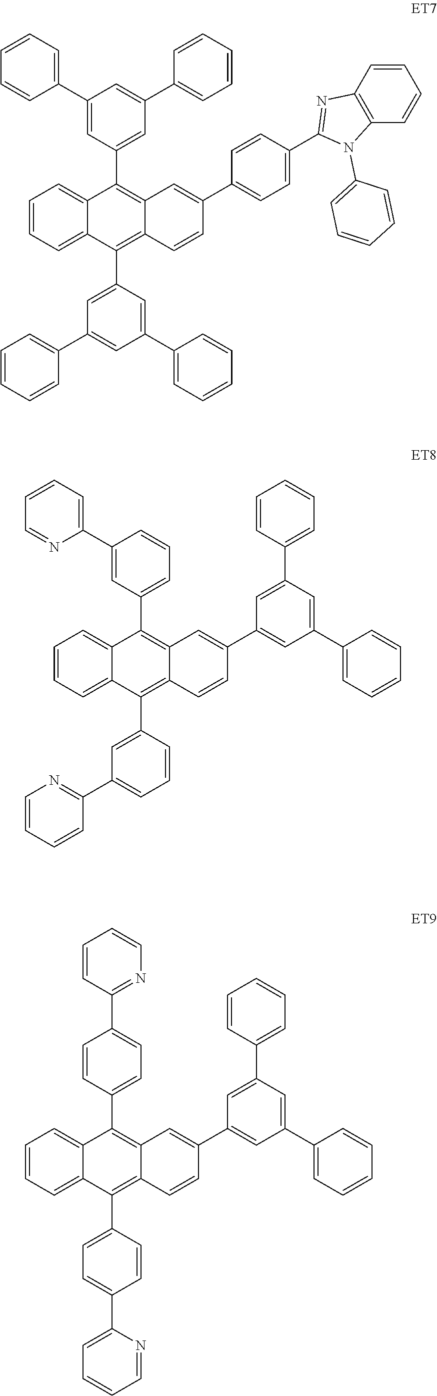 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

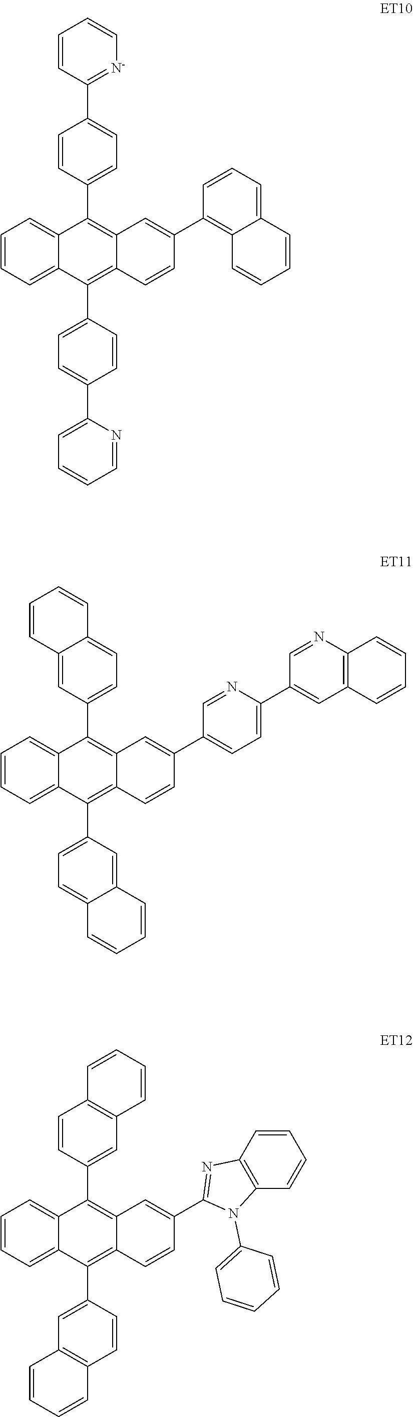

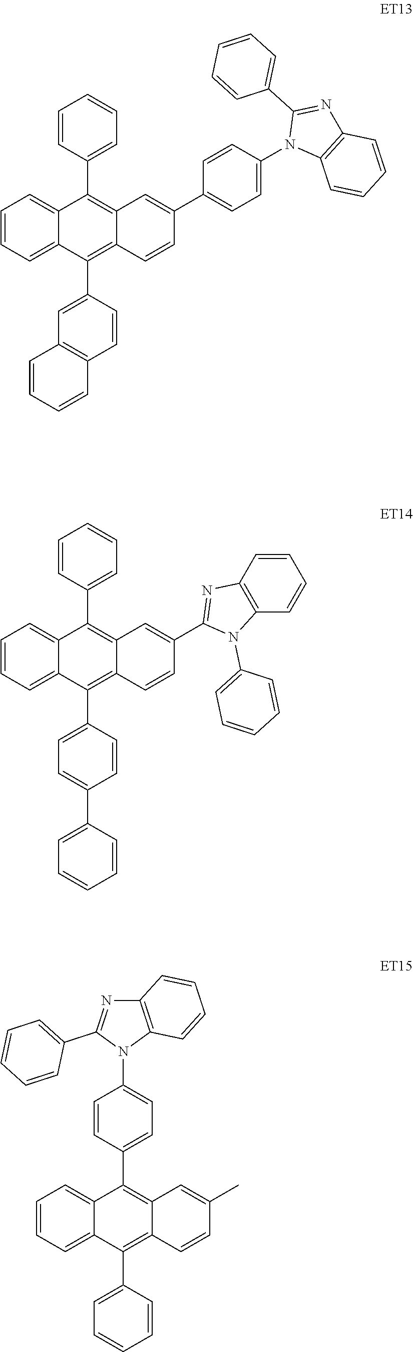 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

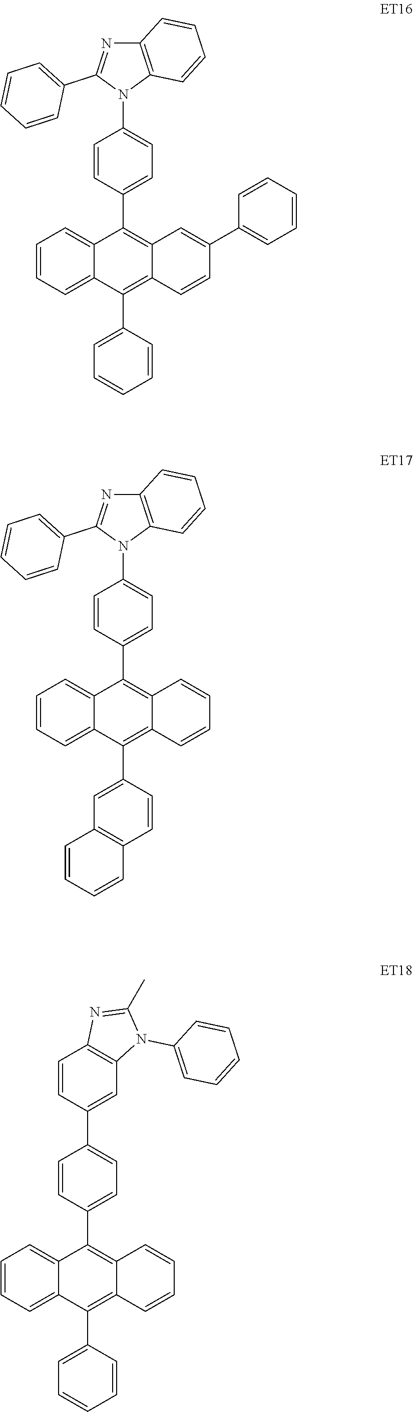 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

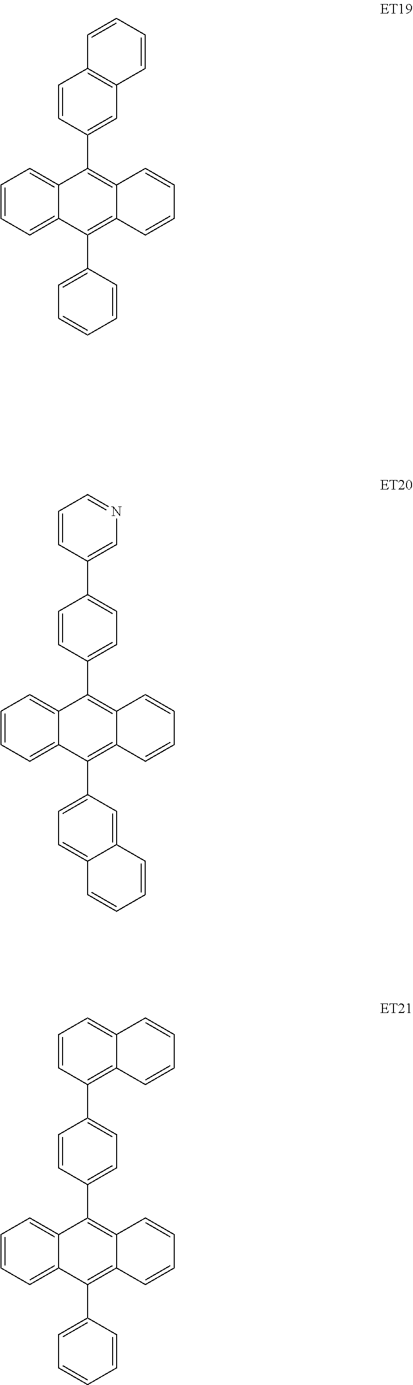

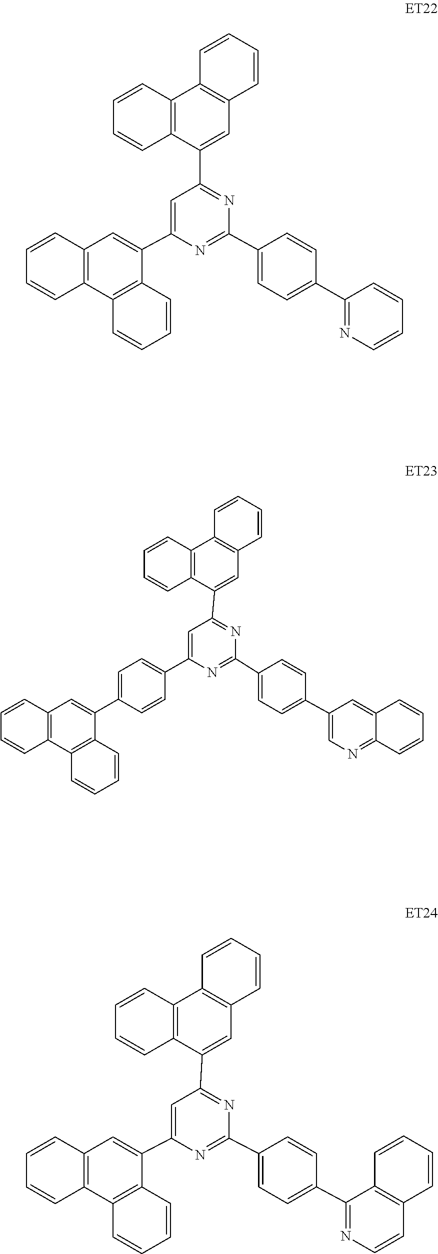 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

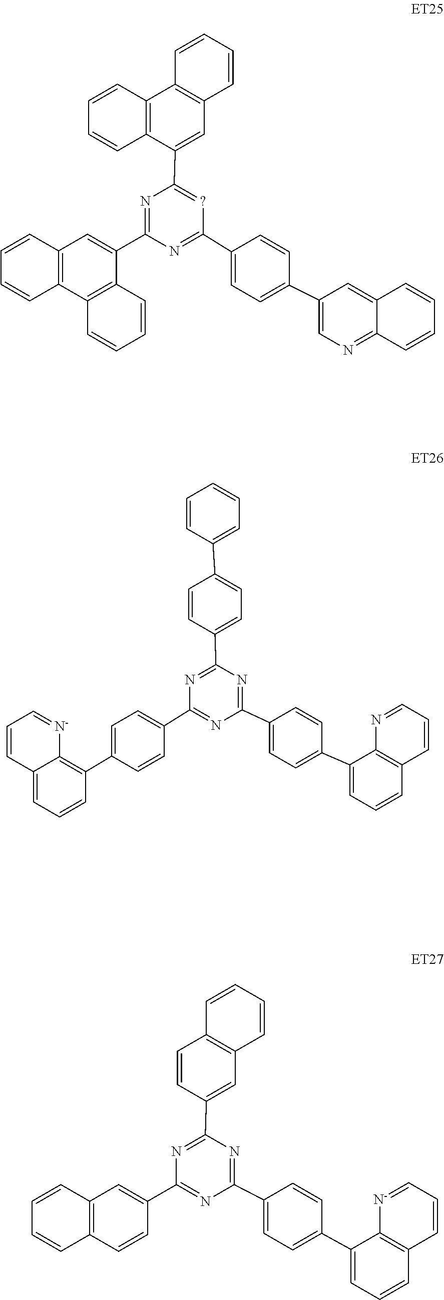

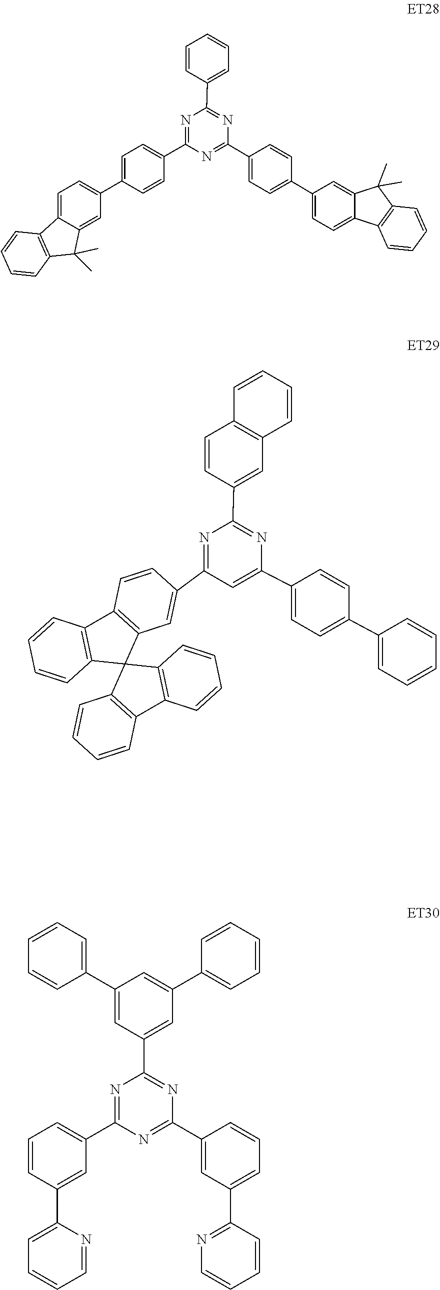 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

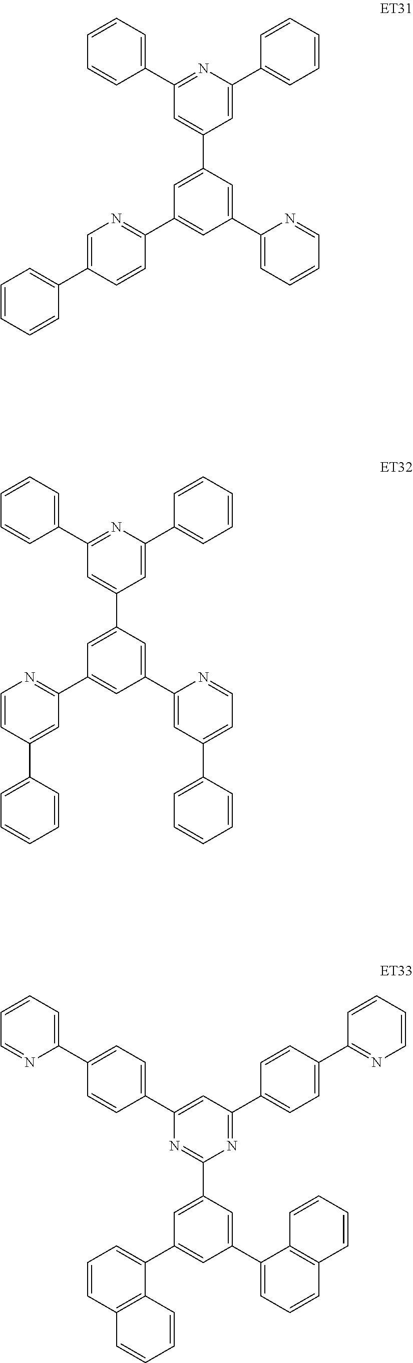 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

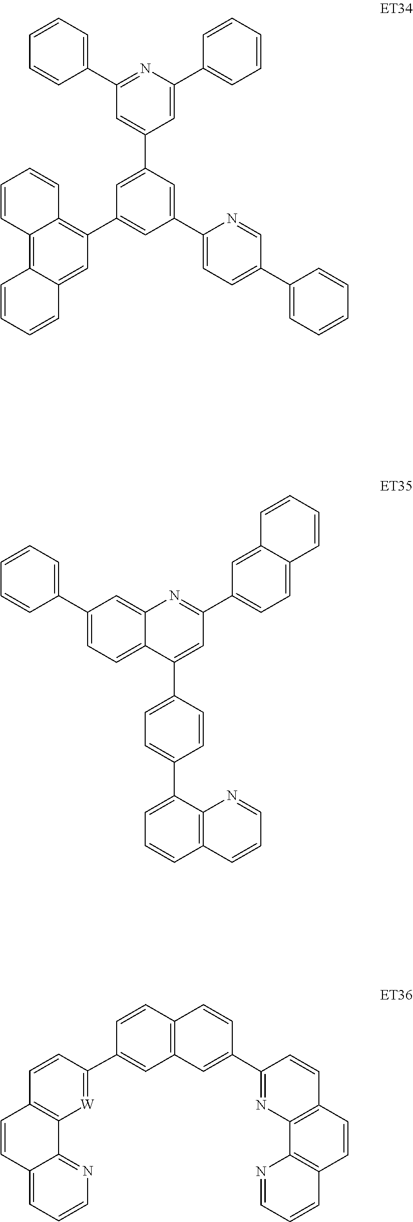

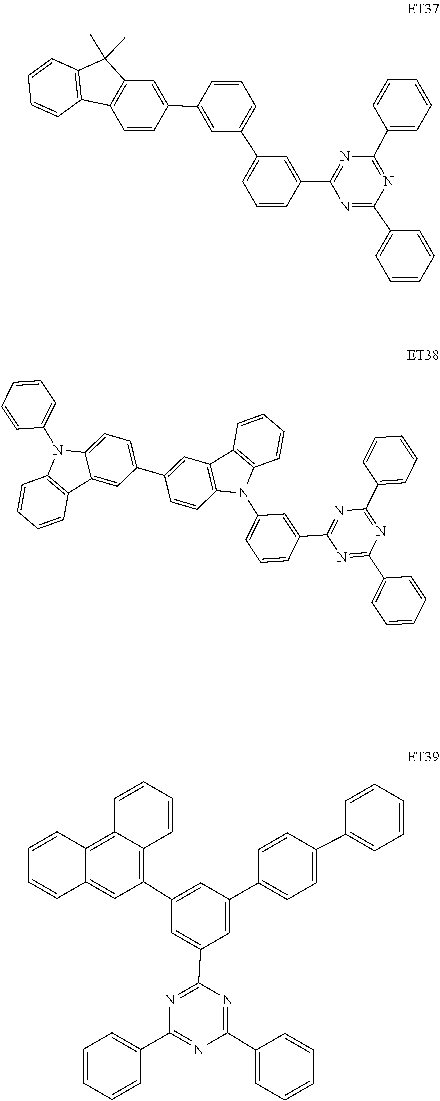 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

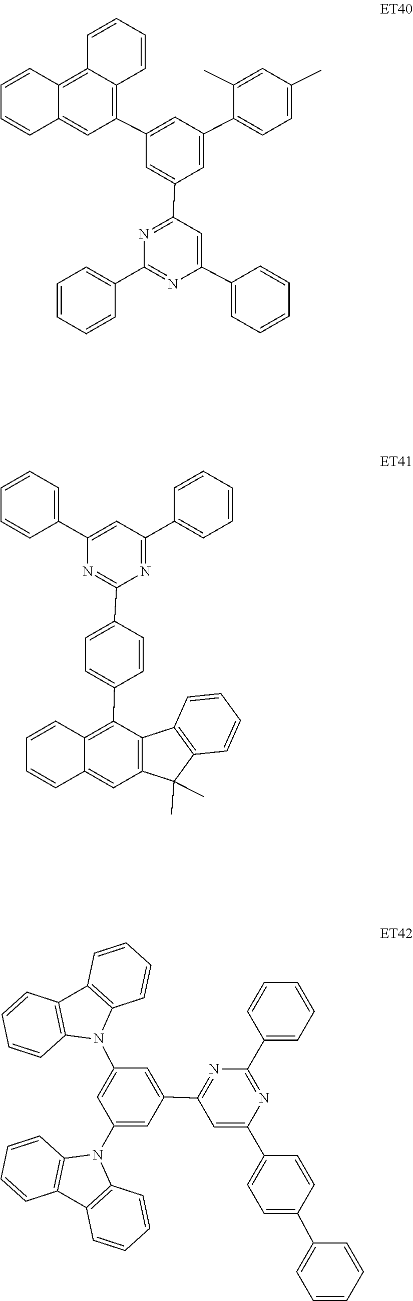 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

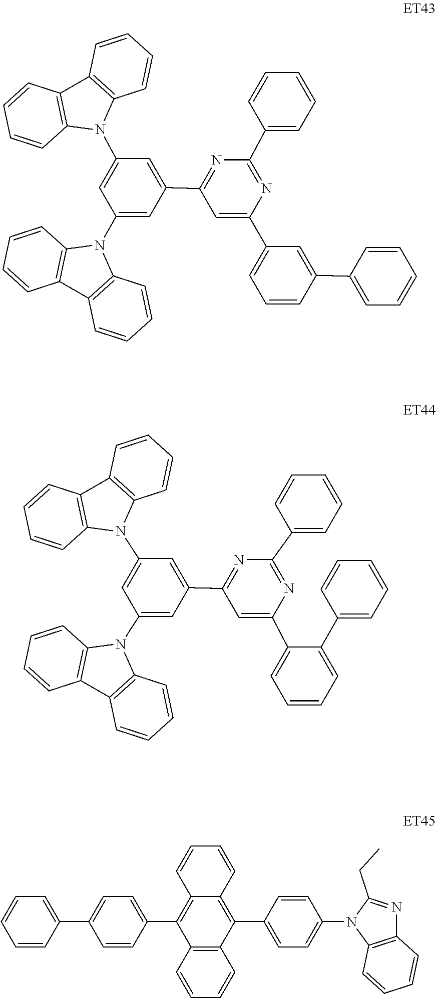 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

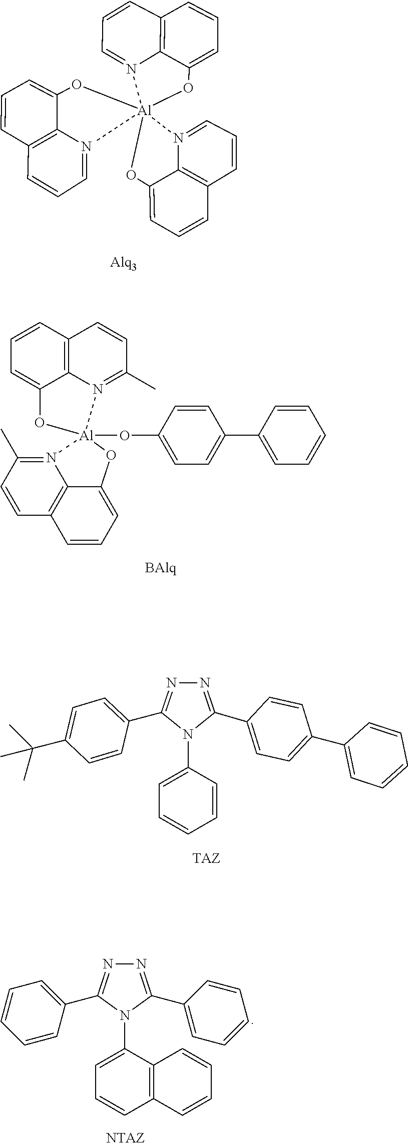

A thickness of the electron transport region 140 may be in a range of about 100 Å to about 5,000 Å, for example, about 160 Å to about 4,000 Å. When the electron transport region 140 includes a buffer layer, a hole-blocking layer, an electron control layer, an electron transport layer, or any combination thereof, a thickness of the buffer layer, the hole-blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole-blocking layer, the electron control layer, the electron transport layer, and/or the electron transport region 140 are within these ranges, satisfactory electron-transporting characteristics may be obtained without a substantial increase in driving voltage.

The electron transport region 140 (e.g., the electron transport layer in the electron transport region 140) may further include, in addition to the materials as described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) or ET-D2:

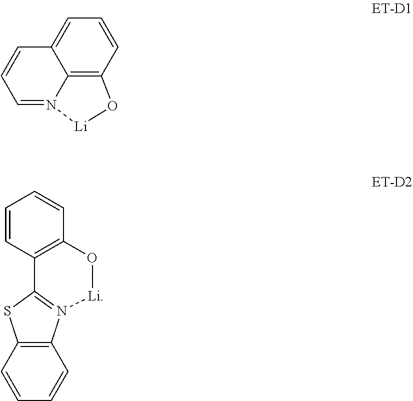

The electron transport region 140 may include an electron injection layer that facilitates injection of electrons from the second electrode 150. The electron injection layer may be in contact with the second electrode 150.

The electron injection layer may have i) a single-layered structure consisting of a single layer consisting of a single material, ii) a single-layered structure consisting of a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (e.g., fluorides, chlorides, bromides, iodides, etc.), or tellurides of the alkali metal, the alkaline earth metal, and the rare earth metal, or any combination thereof.

The alkali metal-containing compound may include an alkali metal oxide, such as Li₂O, Cs₂O, or K₂O, an alkali metal halide, such as LiF, NaF, CsF, KF, LiI, NaI, CsI, or KI, or any combination thereof. The alkaline earth metal-containing compound may include an alkaline earth metal compound, such as BaO, SrO, CaO, BaₓSr₁₋ₓO (wherein x is a real number satisfying the condition of 0<x<1), or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include i) one of ions of the alkali metal, the alkaline earth metal, and the rare earth metal and ii), as a ligand linked to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In an embodiment, the electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In an embodiment, the electron injection layer may consist of i) an alkali metal-containing compound (e.g., an alkali metal halide), or ii) a) an alkali metal-containing compound (e.g., an alkali metal halide); and b) an alkali metal, an alkaline earth metal, a rare earth metal, or any combination thereof. For example, the electron injection layer may be a KI:Yb co-deposited layer, a RbI:Yb co-deposited layer, a LiF:Yb co-deposited layer, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

[Second Electrode 150]

The second electrode 150 may be arranged on the electron transport region 140. The second electrode 150 may be a cathode, which is an electron injection electrode, and as a material for the second electrode 150, a metal, an alloy, an electrically conductive compound, or any combination thereof, each having a low work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

[Capping Layer]

A first capping layer may be arranged outside the first electrode 110, and/or a second capping layer may be arranged outside the second electrode 150. In detail, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order.

In an embodiment, light generated in the emission layer 130 of the light-emitting device 10 may be extracted toward the outside through the first electrode 110, which is a semi-transmissive electrode or a transmissive electrode, and the first capping layer. In one or more embodiments, light generated in the emission layer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In one or more embodiments, at least one of the first capping layer and the second capping layer may each independently include one of Compounds HT28 to HT33, one of Compounds CP1 to CP6, β-NPB, or any combination thereof:

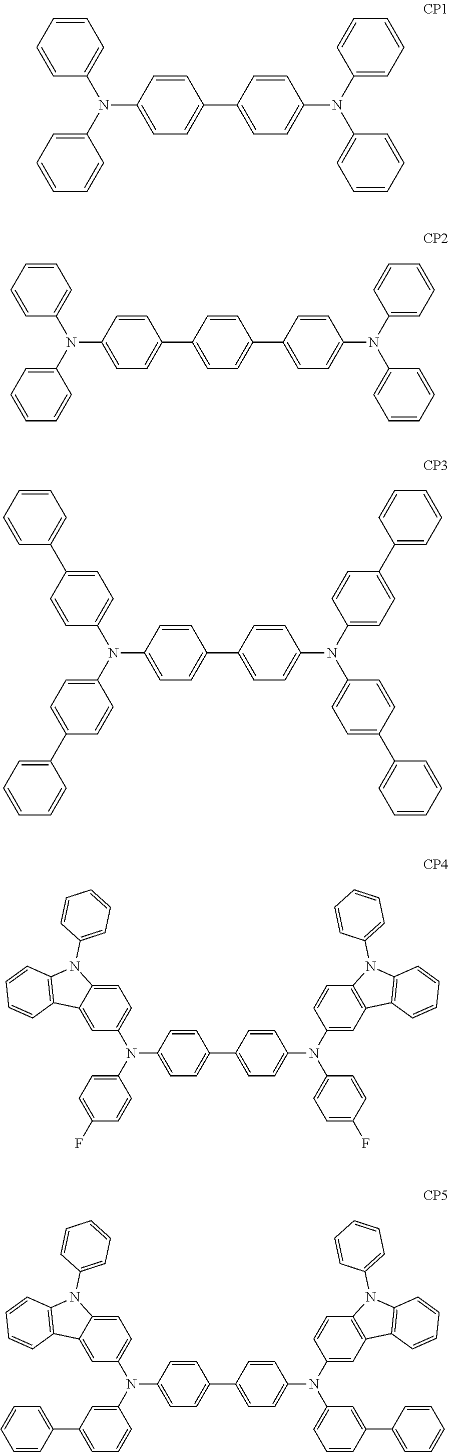 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

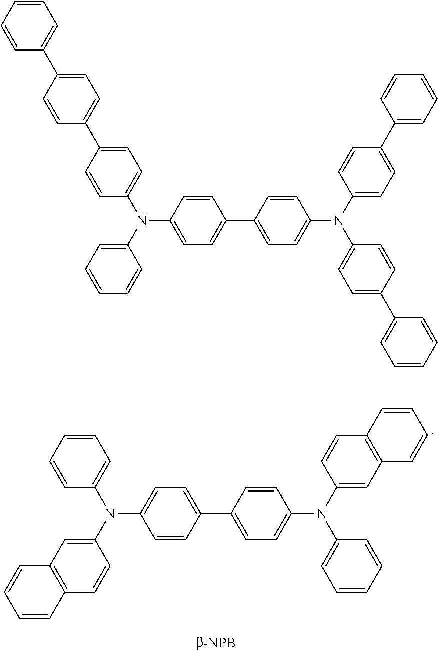 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

[Film]

According to another aspect of the disclosure, the electronic apparatus may include a film. The film may be, for example, an optical member (or a light control member) (e.g., a color filter, a color conversion member, a capping layer, a light-extraction efficiency improvement layer, a selective light-absorbing layer, a polarizing layer, a quantum dot-containing layer, etc.), a light-blocking member (e.g., a light-reflective layer, a light-absorbing layer, etc.), a protection member (e.g. an insulating layer, a dielectric layer, etc.), or the like.

[Electronic Apparatus]

The opto-electronic device 30 may be included in various electronic devices. For example, the electronic apparatus including the opto-electronic device 30 may be a light-emitting apparatus, an authentication apparatus, or the like.

The electronic apparatus (e.g., a light-emitting apparatus) may further include, in addition to the opto-electronic device 30, i) a color filter, ii) a color conversion layer, or iii) a color filter and a color conversion layer. The color filter and/or the color conversion layer may be arranged in at least one direction in which light emitted from the light-emitting device 10 travels. For example, the light emitted from the light-emitting device 10 may be blue light or white light. Details on the light-emitting device 10 may be as described above. In an embodiment, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The color filter areas (or the color conversion areas) may include a first area emitting first-color light, a second area emitting second-color light, and/or a third area emitting third-color light, wherein the first-color light, the second-color light, and/or the third-color light may have different maximum emission wavelengths. For example, the first-color light may be red light, the second-color light may be green light, and the third-color light may be blue light. For example, the color filter areas (or the color conversion areas) may include quantum dots. In detail, the first area may include a red quantum dot, the second area may include a green quantum dot, and the third area may not include a quantum dot. Details on the quantum dot may be as described herein. The first area, the second area, and/or the third area may each further include a scatterer.

For example, the light-emitting device 10 may emit first light, the first area may absorb the first light to emit first first-color light, the second area may absorb the first light to emit second first-color light, and the third area may absorb the first light to emit third first-color light. In this regard, the first first-color light, the second first-color light, and the third first-color light may have different maximum emission wavelengths. In detail, the first light may be blue light, the first first-color light may be red light, the second first-color light may be green light, and the third first-color light may be blue light.

The electronic apparatus may further include a thin-film transistor, in addition to the opto-electronic device 30 described above. The thin-film transistor may include a source electrode, a drain electrode, and an active layer, and one of the source electrode and the drain electrode may be electrically connected to one of the first electrode 110 and the second electrode 150 of the opto-electronic device 30.

The thin-film transistor may further include a gate electrode, a gate insulating film, and the like.

The active layer may include crystalline silicon, amorphous silicon, an organic semiconductor, an oxide semiconductor, and the like.

The electronic apparatus may further include a sealing portion for sealing the opto-electronic device 30. The sealing portion may be arranged between the color filter and/or the color conversion layer and the opto-electronic device 30. The sealing portion may allow light to be incident on the opto-electronic device 30, and may simultaneously prevent ambient air and moisture from penetrating into the opto-electronic device 30. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

Various functional layers may be additionally arranged on the sealing portion, in addition to the color filter and/or the color conversion layer, according to the use of the electronic apparatus. Examples of the functional layer may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (e.g., fingertips, pupils, etc.).

The authentication apparatus may further include, in addition to the light-emitting device 10 as described above, a biometric information collector.

The electronic apparatus may be applied to various displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (e.g., meters for a vehicle, an aircraft, and a vessel), projectors, and the like.

[Electronic Device]

The electronic apparatus including the opto-electronic device 30 may be included in various electronic devices.

For example, the electronic device including the electronic apparatus may be one of a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, and a signboard.

Since the opto-electronic device 30 has excellent photoelectric characteristics, the electronic device including the opto-electronic device 30 may have high-quality characteristics.

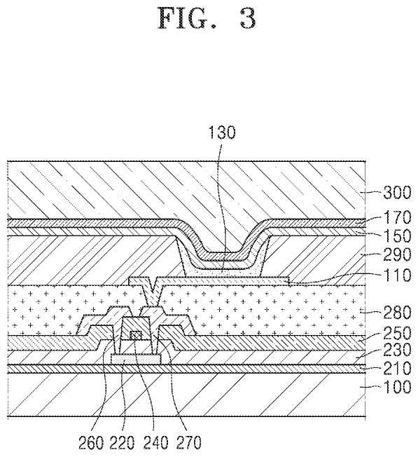

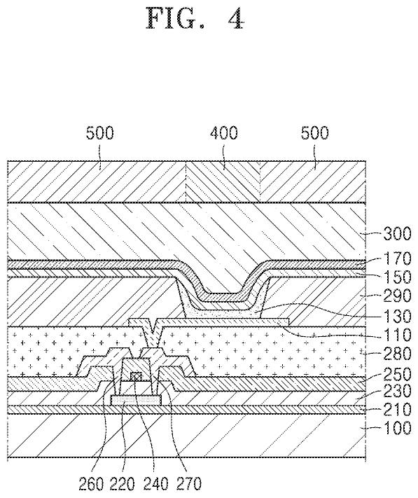

### Description of FIGS. 3 and 4

FIG. 3 is a cross-sectional view of an electronic apparatus according to an embodiment.

The electronic apparatus of FIG. 3 may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be arranged on the substrate 100. The buffer layer 210 may prevent penetration of impurities through the substrate 100, and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose the source region and the drain region of the active layer 220, and the source electrode 260 and the drain electrode 270 may be in contact with the exposed portions of the source region and the drain region of the active layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include a first electrode 110, an emission layer 130, and a second electrode 150.

The first electrode 110 may be arranged on the passivation layer 280. The passivation layer 280 may expose a portion of the drain electrode 270, not fully covering the drain electrode 270, and the first electrode 110 may be connected to the exposed portion of the drain electrode 270.

A pixel-defining film 290 including an insulating material may be arranged on the first electrode 110. The pixel-defining film 290 may expose a portion of the first electrode 110, and the emission layer 130 may be formed in the exposed portion of the first electrode 110. The pixel-defining film 290 may be a polyimide or polyacrylic organic film. Although not shown in FIG. 3, at least some layers of the emission layer 130 may extend beyond the upper portion of the pixel-defining film 290 to be arranged in the form of a common layer.

The second electrode 150 may be arranged on the emission layer 130, and a capping layer 170 may be additionally formed on the second electrode 150. The capping layer 170 may be formed to cover the second electrode 150.

The encapsulation portion 300 may be arranged on the capping layer 170. The encapsulation portion 300 may be arranged on a light-emitting device to protect the light-emitting device from moisture or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (e.g., polymethyl methacrylate, polyacrylic acid, etc.), an epoxy-based resin (e.g., aliphatic glycidyl ether (AGE), etc.), or any combination thereof; or a combination of the inorganic film and the organic film.

FIG. 4 is a cross-sectional view of an electronic apparatus according to another embodiment.

The electronic apparatus of FIG. 4 is the same as the electronic apparatus of FIG. 3, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In an embodiment, the light-emitting device included in the electronic apparatus of FIG. 3 may be a tandem light-emitting device.

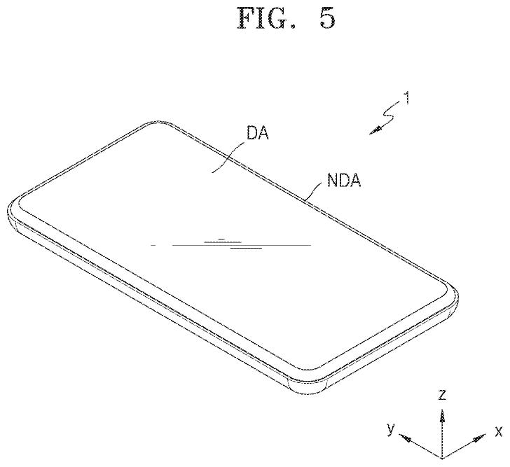

### Description of FIG. 5

FIG. 5 is a schematic perspective view of an electronic device 1 according to an embodiment. The electronic device 1 may be an apparatus that displays a moving image or a still image, and may include a portable electronic device, such as a mobile phone, a smart phone, a tablet personal computer (PC), a mobile communication terminal, an electronic notebook, an electronic book, a portable multimedia player (PMP), a navigation device, or an ultra mobile PC (UMPC), as well as various products, such as a television, a laptop, a monitor, a billboard, or an internet of things (IOT) device, or a part thereof. In addition, the electronic device 1 may be a wearable device, such as a smart watch, a watch phone, a glasses-type display, or a head mounted display (HMD), or a part thereof. However, embodiments are not limited thereto. For example, the electronic device 1 may be an instrument panel of a vehicle, a center information display (CID) arranged on a center fascia or a dashboard of a vehicle, a room mirror display that replaces a side mirror of a vehicle, an entertainment display for a rear seat of a vehicle or a display arranged on a rear surface of a front seat, a head up display (HUD) installed at a front of a vehicle or projected on a front window glass, or a computer generated hologram augmented reality head up display (CGH AR HUD). FIG. 5 shows a case in which the electronic device 1 is a smart phone, for convenience of description.

The electronic device 1 may include a display area DA and a non-display area NDA outside the display area DA. The electronic device 1 may implement an image through an array of a plurality of pixels that are two-dimensionally arranged in the display area DA.

The non-display area NDA is an area in which an image is not displayed, and may entirely surround the display area DA. A driver for providing electrical signals or power to display elements arranged in the display area DA may be arranged in the non-display area NDA. A pad, which is an area to which an electronic element or a printed circuit board may be electrically connected, may be arranged in the non-display area NDA.

The electronic device 1 may have different lengths in the x-axis direction and in the y-axis direction. For example, as shown in FIG. 5, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

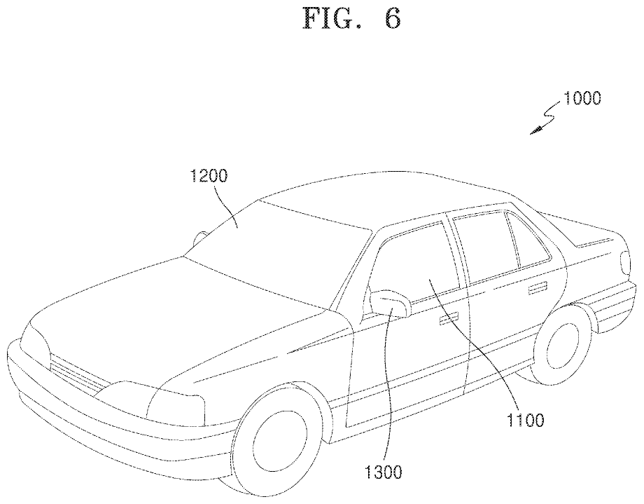

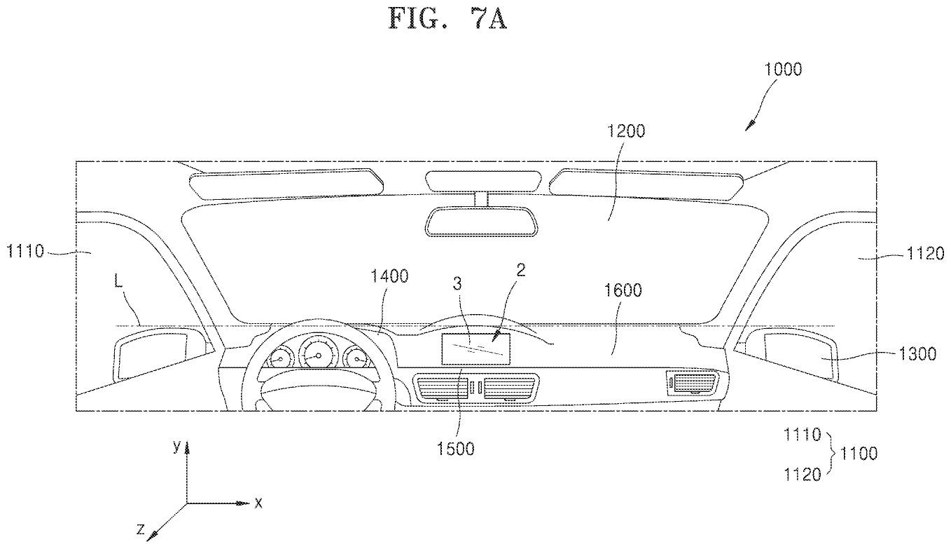

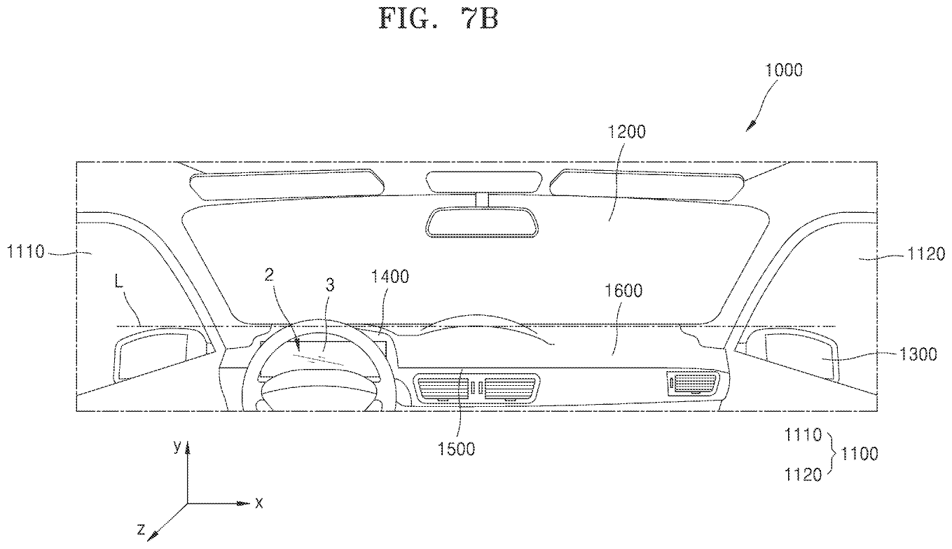

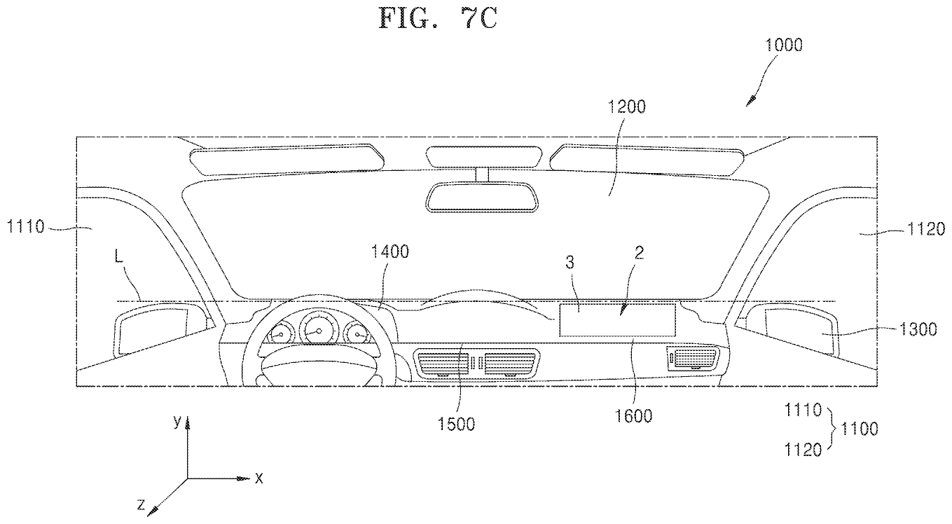

### Description of FIGS. 6 and 7A to 7C

FIG. 6 is a schematic view of the exterior of a vehicle 1000 as an electronic device according to another embodiment. FIGS. 7A to 7C are schematic views of the interior of the vehicle 1000 of FIG. 6.

Referring to FIGS. 6, 7A, 7B, and 7C, the vehicle 1000 may refer to various apparatuses for moving a subject to be transported, such as a human, an object, or an animal, from a departure point to a destination. The vehicle 1000 may include a vehicle traveling on land such as on a road or a track, a vessel moving over water such as the sea or a river, an airplane flying in the sky by using the action of air, and the like.

The vehicle 1000 may travel on a road or a track. The vehicle 1000 may move in a certain direction according to rotation of at least one wheel. For example, the vehicle 1000 may include a three-wheeled or four-wheeled vehicle, a construction machine, a two-wheeled vehicle, a prime mover apparatus, a bicycle, and a train running on a track.

The vehicle 1000 may include a body having an interior and an exterior, and a chassis in which mechanical apparatuses necessary for driving are installed as the remaining parts except for the body. The exterior of the body may include a front panel, a bonnet, a roof panel, a rear panel, a trunk, and a pillar provided at a boundary between doors. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In an embodiment, the side window glass 1100 may be installed on a door of the vehicle 1000. A plurality of side window glasses 1100 may be provided and may face each other. In an embodiment, the side window glass 1100 may include a first side window glass 1110 and a second side window glass 1120. In an embodiment, the first side window glass 1110 may be arranged adjacent to the cluster 1400. The second side window glass 1120 may be arranged adjacent to the passenger seat dashboard 1600.

In an embodiment, the side window glasses 1100 may be apart from each other in the x-direction or the −x-direction. For example, the first side window glass 1110 and the second side window glass 1120 may be apart from each other in the x direction or the −x direction. In other words, an imaginary straight line L connecting the side window glasses 1100 to each other may extend in the x direction or the −x direction. For example, the imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a view of the area around the vehicle 1000. The side mirror 1300 may be installed on the exterior of the body. In an embodiment, a plurality of side mirrors 1300 may be provided. One of the plurality of side mirrors 1300 may be arranged outside the first side window glass 1110. Another one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn indicator, a high beam indicator, a warning lamp, a seat belt warning lamp, an odometer, a hodometer, an automatic transmission selection lever indicator, a door open warning lamp, an engine oil warning lamp, and/or a low fuel warning lamp.

The center fascia 1500 may include a control panel on which a plurality of buttons for adjusting an audio apparatus, an air conditioning apparatus, and a heater of a seat are arranged. The center fascia 1500 may be arranged on one side of the cluster 1400.

The passenger seat dashboard 1600 may be apart from the cluster 1400 with the center fascia 1500 therebetween. In an embodiment, the cluster 1400 may be arranged to correspond to a driver seat (not shown), and the passenger seat dashboard 1600 may be arranged to correspond to a passenger seat (not shown). In an embodiment, the cluster 1400 may be adjacent to the first side window glass 1110, and the passenger seat dashboard 1600 may be adjacent to the second side window glass 1120.

In an embodiment, the display apparatus 2 may include a display panel 3, and the display panel 3 may display an image. The display apparatus 2 may be arranged inside the vehicle 1000. In an embodiment, the display apparatus 2 may be arranged between the side window glasses 1100 facing each other. The display apparatus 2 may be arranged on at least one of the cluster 1400, the center fascia 1500, and the passenger seat dashboard 1600.

The display apparatus 2 may include an organic light-emitting display apparatus, an inorganic light-emitting display apparatus, a quantum dot display apparatus, and the like. Hereinafter, as the display apparatus 2 according to an embodiment, an organic light-emitting display apparatus including the light-emitting device according to an embodiment will be described as an example, but various types of display apparatuses as described above may be used in embodiments.

Referring to FIG. 7A, the display apparatus 2 may be arranged on the center fascia 1500. In an embodiment, the display apparatus 2 may display navigation information. In an embodiment, the display apparatus 2 may display audio, video, or information regarding vehicle settings.

Referring to FIG. 7B, the display apparatus 2 may be arranged on the cluster 1400. In this case, the cluster 1400 may display driving information and the like through the display apparatus 2. That is, the cluster 1400 may be digitally implemented. The cluster 1400 that is digitally implemented may display vehicle information and driving information as images. For example, a needle and a gauge of a tachometer and various warning lamp icons may be displayed by digital signals.

Referring to FIG. 7C, the display apparatus 2 may be arranged on the passenger seat dashboard 1600. The display apparatus 2 may be embedded in the passenger seat dashboard 1600 or arranged on the passenger seat dashboard 1600. In an embodiment, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information that is different from information displayed on the cluster 1400 and/or information displayed on the center fascia 1500.

The locations of the display apparatus 2 in FIG. 7A, FIG. 7B, and FIG. 7C are not mutually exclusive. In some embodiments, there may be multiple display apparatuses in two or three of the locations depicted in FIG. 7A, FIG. 7B, and FIG. 7C.

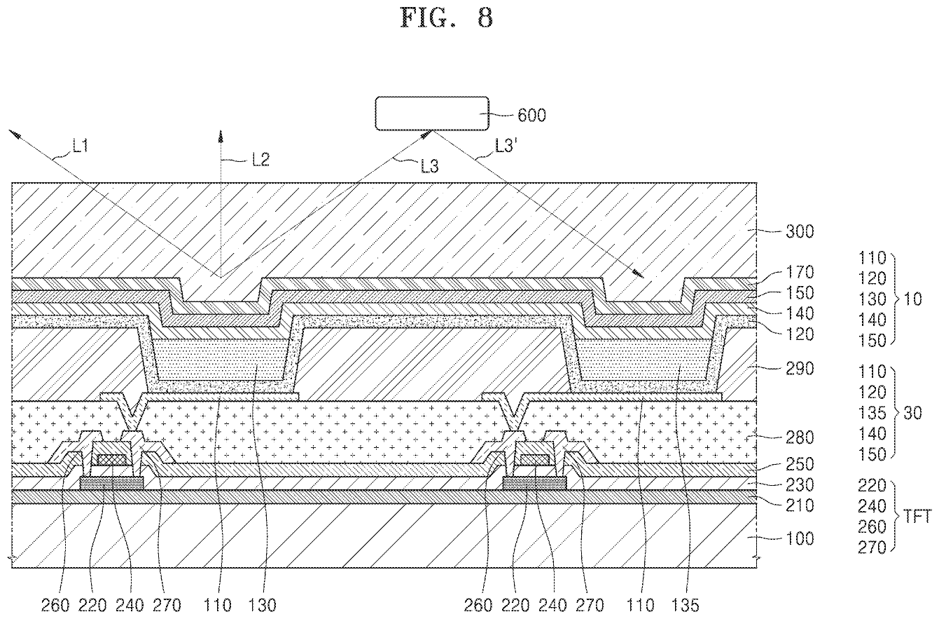

### Descriptions of FIG. 8

FIG. 8 is a cross-sectional view of an electronic apparatus according to an embodiment,

The electronic apparatus of FIG. 8 includes a substrate 100, a thin-film transistor (TFT), a light-emitting device 10, an optoelectronic device 30, and an encapsulation unit 300. Each component shown in FIG. 8 may correspond to each component shown in FIGS. 1 to 4, but is not limited thereto.

The light-emitting device 10 and the optoelectronic device 30 may be disposed on the TFT. The light-emitting device 10 may include the first electrode 110, the hole transport region 120, the emission layer 130, the electron transport region 140, and the second electrode 150. The optoelectronic device 30 may include the first electrode 110, the hole transport region 120, the photoactive layer 135, the electron transport region 140, and the second electrode 150.

The hole transport region 120 may be disposed on the pixel-defining layer 290. The hole transport region 120 included in the light-emitting device 10 may be integrated in one body with the hole transport region 120 included in the optoelectronic device 30. The hole transport region 120 included in the light-emitting device 10 and the hole transport region 120 included in the optoelectronic device 30 may be disposed on the pixel-defining layer 290, may be connected to each other and include substantially the same material, and may be formed substantially at the same time.

The emission layer 130 and the photoactive layer 135 may be disposed on the hole transport region 120. Each of the emission layer 130 and the photoactive layer 135 may overlap the first electrode 110 exposed by the pixel-defining layer 290.

The electron transport region 140 may be disposed on the emission layer 130 and the photoactive layer 135. The electron transport region 140 included in the light-emitting device 10 may be integrated in one body with the electron transport region 140 included in the optoelectronic device 30. The electron transport region 140 included in the light-emitting device 10 and the electron transport region 140 included in the optoelectronic device 30 may be disposed on the pixel-defining layer 290, may be connected to each other and include substantially the same material, and may be formed substantially at the same time.

The second electrode 150 may be disposed on the electron transport region 140. The second electrode 150 included in the light-emitting device 10 may be integrated in one body with the second electrode 150 included in the optoelectronic device 30. The second electrode 150 included in the light-emitting device 10 and the second electrode 150 included in the optoelectronic device 30 may be disposed on the pixel-defining layer 290, may be connected to each other and include substantially the same material, and may be formed substantially at the same time.

The light-emitting device 10 may emit light beams L1, L2, and L3. For example, light beams L1, L2, and L3 may be red light, green light, blue light, or near-infrared light.

A part L3 of the emitted light beams L1, L2, and L3 may be incident on an object 600 outside the electronic apparatus. For example, the object 600 may be a finger of a user of the electronic apparatus. Light L3′ reflected from the object 600 may be incident on the optoelectronic device 30.

The photoactive layer 135 may form excitons by absorbing the incident light L3′. The excitons are capable of generating holes and electrons. That is, the photoactive layer 135 may generate electrical signals by absorbing light. In one or more embodiments, the first compound included in the photoactive layer 135 may serve as a donor for supplying electrons, and the second compound included in the photoactive layer 135 may serve as an acceptor for receiving electrons. That is, the optoelectronic device 30 may detect energy of the light L3′ and convert the same into an electrical signal. Accordingly, the optoelectronic device 30 may recognize the object 600 in contact with (or approaching) the electronic apparatus. Thus, the optoelectronic device 30 including the photoactive layer 135 may serve as an optical sensor (e.g., a fingerprint recognition sensor).

[Manufacturing Method]

Respective layers included in the hole transport region 120, the emission layer 130, the photoactive layer 135, and respective layers included in the electron transport region 140 may be formed in a certain region by using one or more suitable methods selected from vacuum deposition, spin coating, casting, Langmuir-Blodgett (LB) deposition, ink-jet printing, laser-printing, laser-induced thermal imaging, and the like.

When the first layer 131 and the second layer 132 included in the photoactive layer 135, respective layers included in the hole transport region 120, the emission layer 130, and respective layers included in the electron transport region 140 are formed by vacuum deposition, the deposition may be performed at a deposition temperature in a range of about 100° C. to about 500° C., at a vacuum degree in a range of about 10⁻⁸ torr to about 10⁻³ torr, and at a deposition rate in a range of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group that has 3 to 60 carbon atoms and consists of only carbon atoms as ring-forming atoms. The term “C₁-C₆₀ heterocyclic group” as used herein refers to a cyclic group that has 1 to 60 carbon atoms and further includes, in addition to carbon atoms, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group may have 3 to 61 ring-forming atoms.

The term “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N=*′ as a ring-forming moiety.

The term “π electron-deficient nitrogen-containing C₁-C₆₀ cyclic group” as used herein refers to a heterocyclic group that has 1 to 60 carbon atoms and includes *—N=*′ as a ring-forming moiety.

For example,
the C₃-C₆₀ carbocyclic group may be i) a group T1 or ii) a condensed cyclic group in which two or more groups T1 are condensed with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and
the π electron-deficient nitrogen-containing C₁-C₆₀ cyclic group may be i) a group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with each other (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.).

The group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group.

The group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The group T3 may be a furan group, a thiophene group, a 1H-pyrrole group, a silole group, or a borole group.

The group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as used herein refers to a group condensed with any cyclic group, a monovalent group, or a polyvalent group (e.g., a divalent group, a trivalent group, a tetravalent group, etc.), depending on the structure of a formula in connection with which the terms are used.

For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆₀ carbocyclic group and the divalent C₁-C₆₀ heterocyclic group may include a C₃-C₁ cycloalkylene group, a C₁-C₁₀ heterocycloalkylene group, a C₃-C₁₀ cycloalkenylene group, a C₁-C₁ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like.

The term “C₁-C₆₀ alkylene group” as used herein refers to a divalent group having the same structure as the C₁-C₆ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof may include an ethenyl group, a propenyl group, a butenyl group, and the like.

The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof may include an ethynyl group, a propynyl group, and the like.

The term “C₂-C₆₀ alkynylene group” as used herein refers to a divalent group having the same structure as the C₂-C₆₀ alkynyl group.

The term “C₁-C₆ alkoxy group” as used herein refers to a monovalent group represented by —OA₁₀₁ (wherein A₁₀₁ is the C₁-C₆₀ alkyl group), and examples thereof may include a methoxy group, an ethoxy group, an isopropyloxy group, and the like.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and the like.

The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that has 1 to 10 carbon atoms and further includes, in addition to carbon atoms, at least one heteroatom as a ring-forming atom, and examples thereof may include a 1,2,3,4-oxatriazolidinyl group, a tetrahydrofuranyl group, a tetrahydrothiophenyl group, and the like.

The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has 3 to 10 carbon atoms and at least one carbon-carbon double bond in the ring thereof and no aromaticity, and examples thereof may include a cyclopentenyl group, a cyclohexenyl group, a cycloheptenyl group, and the like.

The term “C₃-C₁₀ cycloalkenylene group” as used herein refers to a divalent group having the same structure as the C₃-C₁ cycloalkenyl group.

The term “C₁-C₁₀ heterocycloalkenyl group” as used herein refers to a monovalent cyclic group that has 1 to 10 carbon atoms, at least one heteroatom as a ring-forming atom, in addition to carbon atoms, and at least one double bond in the ring thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like.

The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of 6 to 60 carbon atoms.

The term “C₆-C₆₀ arylene group” as used herein refers to a divalent group having a carbocyclic aromatic system of 6 to 60 carbon atoms.

Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like.

When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group that has a heterocyclic aromatic system of 1 to 60 carbon atoms and further includes, in addition to carbon atoms, at least one heteroatom as a ring-forming atom.

The term “C₁-C₆₀ heteroarylene group” as used herein refers to a divalent group that has a heterocyclic aromatic system of 1 to 60 carbon atoms and further includes, in addition to carbon atoms, at least one heteroatom as a ring-forming atom.

Examples of the C₁-C₆ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like.

When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group that has two or more rings condensed with each other, only carbon atoms (e.g., 8 to 60 carbon atoms) as ring-forming atoms, and no aromaticity in its entire molecular structure. Examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, and the like.

The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group that has two or more rings condensed with each other, at least one heteroatom other than carbon atoms (e.g., 1 to 60 carbon atoms), as a ring-forming atom, and no aromaticity in its entire molecular structure. Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, a benzothienodibenzothiophenyl group, and the like.

The term “divalent non-aromatic condensed heteropolycyclic group” as used herein refers to a divalent group having the same structure as the monovalent non-aromatic condensed heteropolycyclic group.

The term “C₆-C₆₀ aryloxy group” as used herein refers to —OA₁₀₂ (wherein A₁₀₂ is the C₆-C₆₀ aryl group).

The term “C₆-C₆₀ arylthio group” as used herein refers to —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as used herein refers to any atom other than a carbon atom. Examples of the heteroatom may include O, S, N, P, Si, B, Ge, Se, or any combination thereof.

The term “third-row transition metal” as used herein may include hafnium (Hf), tantalum (Ta), tungsten (W), rhenium (Re), osmium (Os), iridium (Ir), platinum (Pt), gold (Au), and the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

The x-axis, y-axis, and z-axis as used herein are not limited to three axes in an orthogonal coordinate system, and may be interpreted in a broad sense including these axes. For example, the x-axis, y-axis, and z-axis may be orthogonal to each other, but the x-axis, y-axis, and z-axis may also refer to different directions that are not orthogonal to each other.

Hereinafter, compounds according to embodiments and light-emitting devices according to embodiments will be described in detail with reference to Synthesis Examples and Examples. The wording “B was used instead of A” used in describing Synthesis Examples means that an identical molar equivalent of B was used in place of A.

### Synthesis Example 1-1 (Synthesis of Compound D1)

Compound D1 was synthesized by referring to Dyes and Pigments (vol 53, published in 2002, pages 57 to 65). The compound thus obtained (0.13 g, 67%) was identified as Compound D1 by ESI-LCMS. (ESI-LCMS: [M]⁺: C₂₄H₁₂BClN₆. 430.09.)

### Synthesis Example 1-2 (Synthesis of Compound D2)

Compound D2 was synthesized by referring to Advanced Functional Materials (Adv. Funct. Mater., published in 2012, vol 22, pages 4322 to 4333). The compound thus obtained (0.19 g, 63%) was identified as Compound D2 by ESI-LCMS. (ESI-LCMS: [M]⁺: C₃₂H₂₀N₄S₅. 620.03.)

### Synthesis Example 1-3 (Synthesis of Compound D3)

Compound D3 was synthesized by referring to KR2021-0091064A. The compound thus obtained (0.15 g, 60%) was identified as Compound D3 by ESI-LCMS. (ESI-LCMS: [M]⁺: C₂₆H₂₁N₃O₃Se. 503.07.)

### Synthesis Example 1-4 (Synthesis of Compound D4)

Compound D4 was synthesized by referring to KR2021-0091064A. The synthesis of Compound D4 was performed in the same manner as used in KR2021-0091064A, except that 2-iodothiophene was used instead of 2-iodoselenophene as the starting material, and 1,3-dimethylpyrimidine-2,4,6(1H,3H,5H)-trione was used instead of 1H-indene-1,3(2H)-dione in the final reaction step. The compound thus obtained (0.15 g, 68%) was identified as Compound D4 by ESI-LCMS. (ESI-LCMS: [M]⁺: C₂₆H₂₁N₃O₃S. 455.13.)

### Synthesis Example 2-1 (Synthesis of Compound A1)

Compound A1 was synthesized by referring to Advanced Materials (vol 23 (2): 268. (doi:10.1002/adma.201001402)). The compound thus obtained (0.15 g, 63%) was identified as Compound A1 by ESI-LCMS. (ESI-LCMS: [M]⁺: C₂₆H₁₂C₁₂N₂O₄. 486.02.)

### Synthesis Example 2-2 (Synthesis of Compound A2)

Compound A2 was synthesized by referring to Advanced Materials (vol 23 (2): 268. (doi:10.1002/adma.201001402)). The compound thus obtained (0.13 g, 68%) was identified as Compound A2 by ESI-LCMS. (ESI-LCMS: [M]⁺: C₂₈H₁₈N₂O₄. 446.13.)

### Synthesis Example 2-3 (Synthesis of Compound A3)

Compound A3 was synthesized by referring to Advanced Materials (vol 23 (2): 268. (doi:10.1002/adma.201001402)). The compound thus obtained (0.14 g, 71%) was identified as Compound A3 by ESI-LCMS. (ESI-LCMS: [M]⁺: C₂₆H₁₄N₂O₄. 418.10.)

### Synthesis Example 2-4 (Synthesis of Compound A4)

Compound A4 was synthesized by referring to Advanced Materials. 23 (2): 268. (doi:10.1002/adma.201001402). The compound thus obtained (0.10 g, 58%) was identified by ESI-LCMS as Compound A4. (ESI-LCMS: [M]⁺: C₁₆H₁₀N₂O₄. 294.06.)

### Synthesis Example 2-5 (Synthesis of Compound A5)

Compound A5 was synthesized by referring to Ullmann's Encyclopedia of Industrial Chemistry, Wiley-VCH, Weinheim, 2012. (doi:10.1002/14356007.a05_249). The compound thus obtained (0.10 g, 72%) was identified by ESI-LCMS as Compound A5. (ESI-LCMS: [M]⁺: C₁₄H₄O₆. 268.00.)

### Synthesis Example 2-6 (Synthesis of Compound A6)

Compound A6 was synthesized by referring to Advanced Materials (vol 23 (2): 268. (doi:10.1002/adma.201001402)). The compound thus obtained (0.16 g, 61%) was identified as Compound A6 by ESI-LCMS. (ESI-LCMS: [M]⁺: C₂₈H₁₂F₆N₂O₄. 552.08.)

### Synthesis Example 2-7 (Synthesis of Compound A7)

Compound A7 was synthesized by referring to Advanced Materials (vol 23 (2): 268. (doi:10.1002/adma.201001402)). The compound thus obtained (0.17 g, 64%) was identified as Compound A7 by ESI-LCMS. (ESI-LCMS: [M]⁺: C₂₈H₁₂F₆N₂O₄. 552.08.)

### Synthesis Example 2-8 (Synthesis of Compound A8)

Compound A8 was synthesized by referring to Advanced Materials (vol 23 (2): 268. (doi:10.1002/adma.201001402)). The compound thus obtained (0.14 g, 65%) was identified as Compound A8 by ESI-LCMS. (ESI-LCMS: [M]⁺: C₂₆H₁₂F₂N₂O₄. 454.08.)

### Synthesis Example 2-9 (Synthesis of Compound A9)

Compound A9 was synthesized by referring to Advanced Materials (vol 23 (2): 268. (doi:10.1002/adma.201001402)). The compound thus obtained (0.13 g, 58%) was identified as Compound A9 by ESI-LCMS. (ESI-LCMS: [M]⁺: C₂₈H₁₂N₄O₄. 468.09.)

### Comparative Example 1-1

As an anode, a glass substrate with 15 Ω/cm² (1,200 Å) ITO formed thereon (manufactured by Corning Inc.) was cut to a size of 50 mm×50 mm×0.7 mm, sonicated using isopropyl alcohol and pure water, each for 5 minutes, cleaned by irradiation of ultraviolet rays and exposure of ozone thereto for 30 minutes, and then mounted on a vacuum deposition apparatus.

2-TNATA was vacuum-deposited on the anode to form a hole injection layer having a thickness of 600 Å. 4,4′-bis[N-(1-naphthyl)-N-phenylamino]biphenyl (hereinafter, referred to as NPB) was vacuum-deposited on the hole injection layer to form a hole transport layer having a thickness of 300 Å.

Compound D1 (subphthalocyanine chloride, SubPC) was vacuum-deposited on the hole transport layer to form a first layer included in a photoactive layer and having a thickness of 200 Å.

Fullerene 60 was vacuum-deposited on the first layer to form a second layer included in the photoactive layer and having a thickness of 250 Å.

Alq₃ was vacuum-deposited on the second layer to form an electron transport layer having a thickness of 300 Å. LiF was vacuum-deposited on the electron transport layer to form an electron injection layer having a thickness of 10 Å. Al was vacuum-deposited on the electron injection layer to form a cathode having a thickness of 3,000 Å, thereby completing the manufacture of an opto-electronic device.

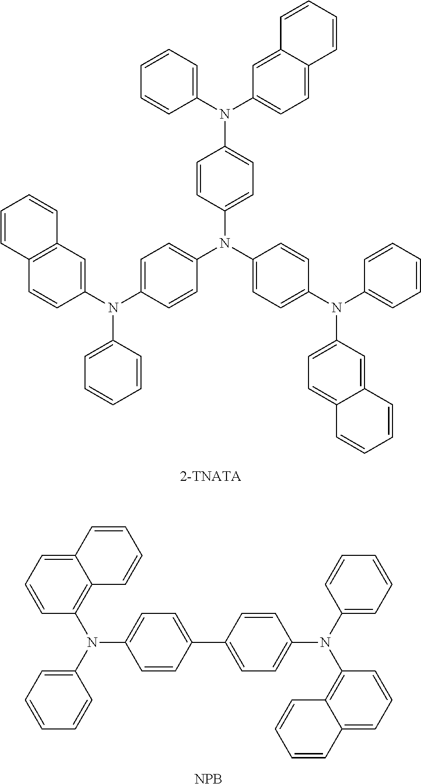 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

### Comparative Examples 1-2 to 1-6, 2-1 to 2-6, 3-1 to 3-6, and 4-1 to 4-6 and Examples 1-1 to 1-9, 2-1 to 2-9, 3-1 to 3-9, and 4-1 to 4-9

Opto-electronic devices were manufactured in the same manner as in Comparative Example 1-1, except that compounds shown in Tables 3 to 6 were respectively used in forming the first layer and the second layer included in the photoactive layer.

### Evaluation Example 1

To evaluate the characteristics of the first compound used in the first layer in each of Comparative Examples 1-1 to 1-6, 2-1 to 2-6, 3-1 to 3-6, and 4-1 to 4-6 and Examples 1-1 to 1-9, 2-1 to 2-9, 3-1 to 3-9, and 4-1 to 4-9, the HOMO energy level (E¹_{HOMO}), LUMO energy level (E¹_{LUMO}), and maximum absorption wavelength (λ_{abs}) were measured, and results thereof are shown in Table 1.

In evaluating the characteristics, quantum simulation was performed at a level of B3LYP/6-311 G** based on the time-dependent density functional theory (TD-DFT) method by using the Gaussian program to evaluate the energy levels and maximum absorption wavelength in a structurally optimized state and an excited state.

TABLE 1

| Compound used | E¹_{HOMO} | E¹_{LUMO} | λ_{abs} |
|---|---|---|---|
| in first layer | (eV) | (eV) | (nm) |
| D1 | −5.6 | −2.8 | 590 |
| D2 | −5.82 | −3.24 | 525 |
| D3 | −5.64 | −2.77 | 555 |
| D4 | −5.66 | −2.74 | 542 |

### Evaluation Example 2

To evaluate the characteristics of the second compound used in the second layer in each of Comparative Examples 1-1 to 1-6, 2-1 to 2-6, 3-1 to 3-6, and 4-1 to 4-6 and Examples 1-1 to 1-9, 2-1 to 2-9, 3-1 to 3-9, and 4-1 to 4-9, the HOMO energy level (E²_{HOMO}), LUMO energy level (E²_{LUMO}), and energy band gap (E_{g}) were measured and/or calculated, and results thereof are shown in Table 2.

The HOMO energy level (E²_{HOMO}) of the second compound was measured using the same measurement method as the HOMO energy level (E¹_{HOMO}) of the first compound.

The LUMO energy level (E²_{LUMO}) of the second compound was measured using the same measurement method as the LUMO energy level (E¹_{LUMO}) of the first compound.

The energy band gap (E_{g}) of the second compound was calculated as the difference between the LUMO energy level (E²_{LUMO}) and the HOMO energy level (E²_{HOMO}).

TABLE 2

| Compound used in | E²_{HOMO} | E²_{LUMO} | E_{g} |
|---|---|---|---|
| second layer | (eV) | (eV) | (eV) |
| Fullerene 60 | −6.40 | −3.66 | 2.74 |
| Fullerene 70 | −6.35 | −3.66 | 2.69 |
| C1 | −6.99 | −4.65 | 2.33 |
| C2 | −6.91 | −4.54 | 2.37 |
| C3 | −8.62 | −5.06 | 3.56 |
| C4 | −9.15 | −5.65 | 3.50 |
| A1 | −7.19 | −3.75 | 3.44 |
| A2 | −6.91 | −3.49 | 3.42 |
| A3 | −7.17 | −3.55 | 3.62 |
| A4 | −7.25 | −3.61 | 3.64 |
| A5 | −7.91 | −4.20 | 3.71 |
| A6 | −7.50 | −3.86 | 3.64 |
| A7 | −7.46 | −3.82 | 3.64 |
| A8 | −7.20 | −3.69 | 3.51 |
| A9 | −7.63 | −3.99 | 3.64 |

### Evaluation Example 3

To evaluate the characteristics of the opto-electronic device manufactured in each of Comparative Examples 1-1 to 1-6, 2-1 to 2-6, 3-1 to 3-6, and 4-1 to 4-6 and Examples 1-1 to 1-9, 2-1 to 2-9, 3-1 to 3-9, and 4-1 to 4-9, E¹_{LUMO}−E²_{LUMO}, E¹_{LUMO}−E¹_{HOMO}, and (E¹_{LUMO}−E²_{LUMO})/(E¹_{LUMO}−E¹_{HOMO}) were calculated, and the external quantum efficiency (EQE) and dark current density (J_{dark}) were measured. Measurement results obtained by using Compound D1 in the first layer are shown in Table 3. Measurement results obtained by using Compound D2 in the first layer are shown in Table 4. Measurement results obtained by using Compound D3 in the first layer are shown in Table 5. Measurement results obtained by using Compound D4 in the first layer are shown in Table 6.

In measuring the external quantum efficiency (EQE), a current value generated when light was irradiated to the manufactured opto-electronic device, by using an external quantum efficiency meter (K3100, McScience, Korea), was measured using an ammeter (Keithley, Tektronix, USA), and the measured current value was calculated as an external quantum efficiency (EQE) value.

In measuring the dark current density (J_{dark}), a voltage was applied to the anode by using an electro-optical characteristics evaluation facility (K3100, McScience, Korea), and the dark current density at a reverse bias of −3 V was measured using an ammeter (Keithley, Tektronix, USA).

TABLE 3

| | Compound | Compound | | | (E¹_{LUMO} − | | |
|---|---|---|---|---|---|---|---|
| | used in | used in | E¹_{LUMO} − | E¹_{LUMO} − | E²_{LUMO})/ | | |
| | first | second | E²_{LUMO} | E¹_{HOMO} | (E¹_{LUMO} − | EQE | J_{dark} |
| | layer | layer | (eV) | (eV) | E¹_{HOMO}) | (%) | (mA/cm²) |
| Comparative | D1 | Fullerene | 0.83 | 2.72 | 0.30 | 20 | 3.5 × 10⁻⁶ |
| Example 1-1 | | 60 | | | | | |
| Comparative | D1 | Fullerene | 0.83 | 2.72 | 0.30 | 25 | 5.0 × 10⁻⁶ |
| Example 1-2 | | 70 | | | | | |
| Comparative | D1 | C1 | 1.82 | 2.72 | 0.67 | 11 | 9.7 × 10⁻⁶ |
| Example 1-3 | | | | | | | |
| Comparative | D1 | C2 | 1.71 | 2.72 | 0.63 | 12 | 1.4 × 10⁻⁵ |
| Example 1-4 | | | | | | | |
| Comparative | D1 | C3 | 2.22 | 2.72 | 0.82 | 9 | 1.7 × 10⁻⁵ |
| Example 1-5 | | | | | | | |
| Comparative | D1 | C4 | 2.82 | 2.72 | 1.04 | 7 | 2.1 × 10⁻⁵ |
| Example 1-6 | | | | | | | |
| Example 1-1 | D1 | A1 | 0.92 | 2.72 | 0.34 | 38 | 5.5 × 10⁻⁶ |
| Example 1-2 | D1 | A2 | 0.66 | 2.72 | 0.24 | 33 | 2.7 × 10⁻⁶ |
| Example 1-3 | D1 | A3 | 0.72 | 2.72 | 0.27 | 25 | 4.8 × 10⁻⁶ |
| Example 1-4 | D1 | A4 | 0.78 | 2.72 | 0.29 | 28 | 4.8 × 10⁻⁶ |
| Example 1-5 | D1 | A5 | 1.37 | 2.72 | 0.50 | 24 | 7.2 × 10⁻⁶ |
| Example 1-6 | D1 | A6 | 1.03 | 2.72 | 0.38 | 29 | 5.6 × 10⁻⁶ |
| Example 1-7 | D1 | A7 | 0.99 | 2.72 | 0.36 | 30 | 4.0 × 10⁻⁶ |
| Example 1-8 | D1 | A8 | 0.86 | 2.72 | 0.32 | 29 | 5.4 × 10⁻⁶ |
| Example 1-9 | D1 | A9 | 1.16 | 2.72 | 0.43 | 27 | 7.5 × 10⁻⁶ |

TABLE 4

| | Compound | Compound | | | (E¹_{LUMO} − | | |
|---|---|---|---|---|---|---|---|
| | used in | used in | E¹_{LUMO} − | E¹_{LUMO} − | E²_{LUMO})/ | | |
| | first | second | E²_{LUMO} | E¹_{HOMO} | (E¹_{LUMO} − | EQE | J_{dark} |
| | layer | layer | (eV) | (eV) | E¹_{HOMO}) | (%) | (mA/cm²) |
| Comparative | D2 | Fullerene | 0.42 | 2.58 | 0.16 | 22 | 3.6 × 10⁻⁶ |
| Example 2-1 | | 60 | | | | | |
| Comparative | D2 | Fullerene | 0.42 | 2.58 | 0.16 | 28 | 5.1 × 10⁻⁶ |
| Example 2-2 | | 70 | | | | | |
| Comparative | D2 | C1 | 1.41 | 2.58 | 0.55 | 12 | 9.8 × 10⁻⁶ |
| Example 2-3 | | | | | | | |
| Comparative | D2 | C2 | 1.30 | 2.58 | 0.50 | 13 | 9.6 × 10⁻⁶ |
| Example 2-4 | | | | | | | |
| Comparative | D2 | C3 | 1.82 | 2.58 | 0.70 | 10 | 1.4 × 10⁻⁵ |
| Example 2-5 | | | | | | | |
| Comparative | D2 | C4 | 2.41 | 2.58 | 0.93 | 8 | 1.8 × 10⁻⁵ |
| Example 2-6 | | | | | | | |
| Example 2-1 | D2 | A1 | 0.51 | 2.58 | 0.20 | 42 | 5.7 × 10⁻⁶ |
| Example 2-2 | D2 | A2 | 0.25 | 2.58 | 0.10 | 36 | 2.7 × 10⁻⁶ |
| Example 2-3 | D2 | A3 | 0.31 | 2.58 | 0.12 | 28 | 4.9 × 10⁻⁶ |
| Example 2-4 | D2 | A4 | 0.37 | 2.58 | 0.14 | 31 | 4.9 × 10⁻⁶ |
| Example 2-5 | D2 | A5 | 0.96 | 2.58 | 0.37 | 26 | 7.4 × 10⁻⁶ |
| Example 2-6 | D2 | A6 | 0.62 | 2.58 | 0.24 | 32 | 5.7 × 10⁻⁶ |
| Example 2-7 | D2 | A7 | 0.58 | 2.58 | 0.22 | 33 | 4.1 × 10⁻⁶ |
| Example 2-8 | D2 | A8 | 0.45 | 2.58 | 0.17 | 32 | 5.5 × 10⁻⁶ |
| Example 2-9 | D2 | A9 | 0.75 | 2.58 | 0.29 | 30 | 7.7 × 10⁻⁶ |

TABLE 5

| | Compound | Compound | | | (E¹_{LUMO} − | | |
|---|---|---|---|---|---|---|---|
| | used in | used in | E¹_{LUMO} − | E¹_{LUMO} − | E²_{LUMO})/ | | |
| | first | second | E²_{LUMO} | E¹_{HOMO} | (E¹_{LUMO} − | EQE | J_{dark} |
| | layer | layer | (eV) | (eV) | E¹_{HOMO}) | (%) | (mA/cm²) |
| Comparative | D3 | Fullerene | 0.89 | 2.86 | 0.31 | 23 | 3.4 × 10⁻⁶ |
| Example 3-1 | | 60 | | | | | |
| Comparative | D3 | Fullerene | 0.89 | 2.86 | 0.31 | 29 | 4.8 × 10⁻⁶ |
| Example 3-2 | | 70 | | | | | |
| Comparative | D3 | C1 | 1.88 | 2.86 | 0.66 | 13 | 9.6 × 10⁻⁶ |
| Example 3-3 | | | | | | | |
| Comparative | D3 | C2 | 1.77 | 2.86 | 0.62 | 14 | 1.4 × 10⁻⁵ |
| Example 3-4 | | | | | | | |
| Comparative | D3 | C3 | 2.28 | 2.86 | 0.80 | 11 | 1.6 × 10⁻⁵ |
| Example 3-5 | | | | | | | |
| Comparative | D3 | C4 | 2.88 | 2.86 | 1.01 | 8 | 2.0 × 10⁻⁵ |
| Example 3-6 | | | | | | | |
| Example 3-1 | D3 | A1 | 0.98 | 2.86 | 0.34 | 45 | 5.4 × 10⁻⁶ |
| Example 3-2 | D3 | A2 | 0.72 | 2.86 | 0.25 | 39 | 2.7 × 10⁻⁶ |
| Example 3-3 | D3 | A3 | 0.78 | 2.86 | 0.27 | 29 | 4.7 × 10⁻⁶ |
| Example 3-4 | D3 | A4 | 0.84 | 2.86 | 0.29 | 33 | 4.8 × 10⁻⁶ |
| Example 3-5 | D3 | A5 | 1.43 | 2.86 | 0.50 | 28 | 7.1 × 10⁻⁶ |
| Example 3-6 | D3 | A6 | 1.08 | 2.86 | 0.38 | 34 | 5.6 × 10⁻⁶ |
| Example 3-7 | D3 | A7 | 1.05 | 2.86 | 0.37 | 35 | 4.0 × 10⁻⁶ |
| Example 3-8 | D3 | A8 | 0.92 | 2.86 | 0.32 | 34 | 5.4 × 10⁻⁶ |
| Example 3-9 | D3 | A9 | 1.22 | 2.86 | 0.42 | 32 | 7.5 × 10⁻⁶ |

TABLE 6

| | Compound | Compound | | | (E¹_{LUMO} − | | |
|---|---|---|---|---|---|---|---|
| | used in | used in | E¹_{LUMO} − | E¹_{LUMO} − | E²_{LUMO})/ | | |
| | first | second | E²_{LUMO} | E¹_{HOMO} | (E¹_{LUMO} − | EQE | J_{dark} |
| | layer | layer | (eV) | (eV) | E¹_{HOMO}) | (%) | (mA/cm²) |
| Comparative | D4 | Fullerene | 0.92 | 2.92 | 0.32 | 22 | 3.2 × 10⁻⁶ |
| Example 4-1 | | 60 | | | | | |
| Comparative | D4 | Fullerene | 0.92 | 2.92 | 0.32 | 28 | 4.7 × 10⁻⁶ |
| Example 4-2 | | 70 | | | | | |
| Comparative | D4 | C1 | 1.91 | 2.92 | 0.65 | 11 | 9.7 × 10⁻⁶ |
| Example 4-3 | | | | | | | |
| Comparative | D4 | C2 | 1.80 | 2.92 | 0.62 | 13 | 1.2 × 10⁻⁵ |
| Example 4-4 | | | | | | | |
| Comparative | D4 | C3 | 2.32 | 2.92 | 0.79 | 10 | 1.5 × 10⁻⁵ |
| Example 4-5 | | | | | | | |
| Comparative | D4 | C4 | 2.91 | 2.92 | 1.00 | 7 | 2.1 × 10⁻⁵ |
| Example 4-6 | | | | | | | |
| Example 4-1 | D4 | A1 | 1.01 | 2.92 | 0.35 | 43 | 5.5 × 10⁻⁶ |
| Example 4-2 | D4 | A2 | 0.75 | 2.92 | 0.26 | 38 | 2.7 × 10⁻⁶ |
| Example 4-3 | D4 | A3 | 0.81 | 2.92 | 0.28 | 29 | 4.6 × 10⁻⁶ |
| Example 4-4 | D4 | A4 | 0.87 | 2.92 | 0.30 | 32 | 4.7 × 10⁻⁶ |
| Example 4-5 | D4 | A5 | 1.46 | 2.92 | 0.50 | 27 | 7.2 × 10⁻⁶ |
| Example 4-6 | D4 | A6 | 1.12 | 2.92 | 0.38 | 33 | 5.7 × 10⁻⁶ |
| Example 4-7 | D4 | A7 | 1.08 | 2.92 | 0.37 | 34 | 4.2 × 10⁻⁶ |
| Example 4-8 | D4 | A8 | 0.95 | 2.92 | 0.33 | 32 | 5.1 × 10⁻⁶ |
| Example 4-9 | D4 | A9 | 1.25 | 2.92 | 0.43 | 30 | 7.8 × 10⁻⁶ |

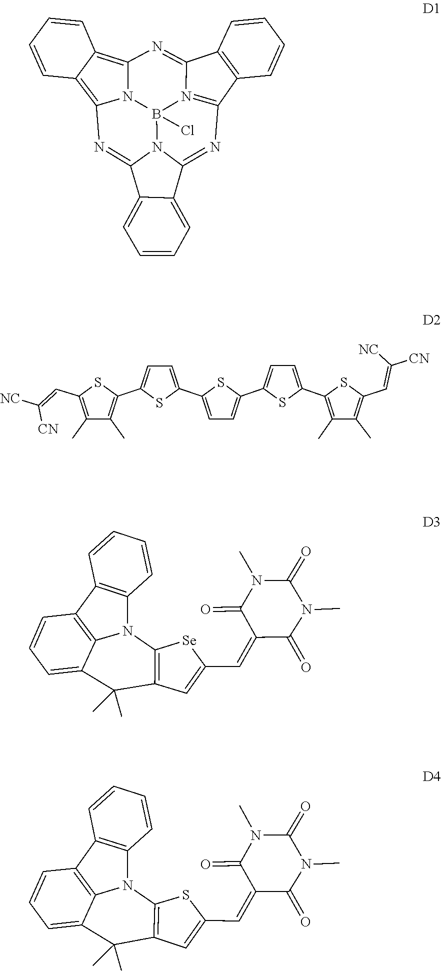

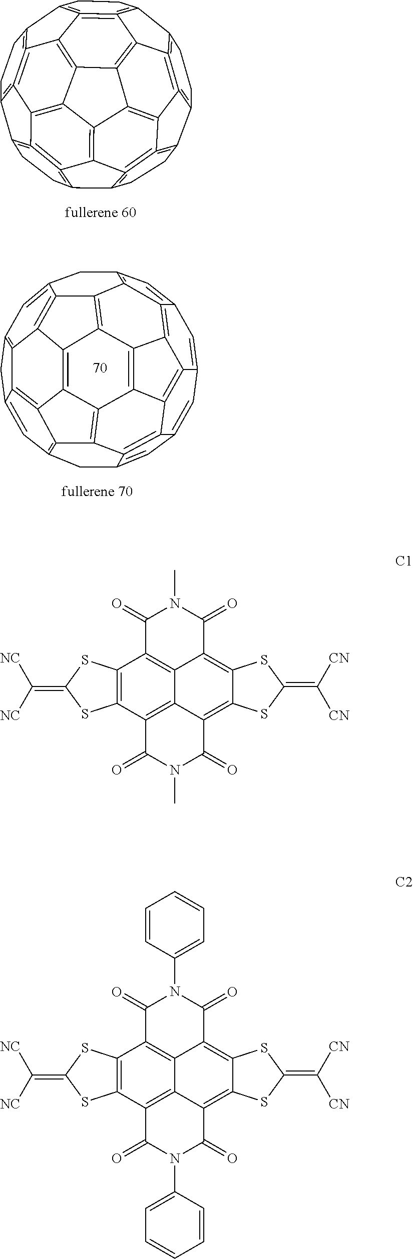

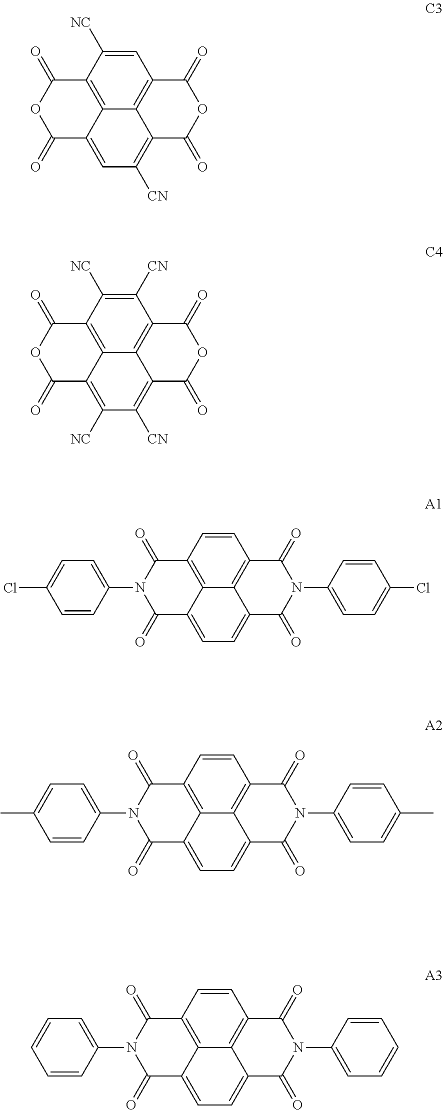

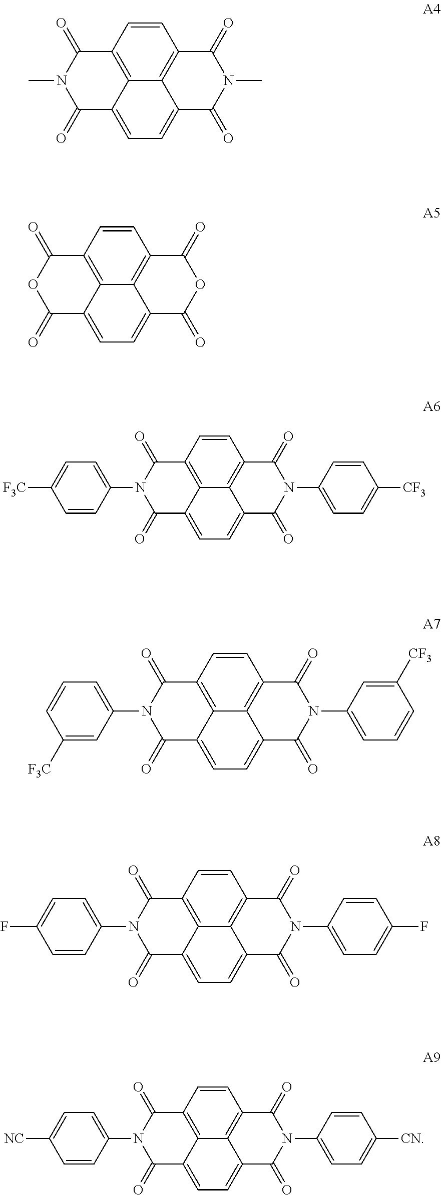

From Tables 3 to 6, it was confirmed that the opto-electronic devices according to Examples 1-1 to 1-9, 2-1 to 2-9, 3-1 to 3-9, and 4-1 to 4-9 had high external quantum efficiency (EQE) and/or low dark current density (J_{dark}), as compared with the opto-electronic devices according to Comparative Examples 1-1 to 1-6, 2-1 to 2-6, 3-1 to 3-6, and 4-1 to 4-6. Thus, it was confirmed that the opto-electronic device according to an embodiment had high photoelectric characteristics and/or low noise.

According to the one or more embodiments, when Equation 1 regarding the relationship between the first compound and the second compound is satisfied, the first compound and the second compound may have excellent exciton-forming characteristics and excellent charge-transfer characteristics. An opto-electronic device including the first compound and the second compound may have excellent photoelectric characteristics.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An opto-electronic device comprising:
a first electrode;
a second electrode facing the first electrode; and
a photoactive layer arranged between the first electrode and the second electrode,
wherein the photoactive layer comprises a first layer and a second layer, the first layer comprising a first compound, and the second layer comprising a second compound that does not comprise a 5-membered ring,
the first compound is an electron-donating compound,
the second compound is an electron-accepting compound, and
the opto-electronic device satisfies Equation 1:
0.1≤(E¹_{LUMO}−E²_{LUMO})/(E¹_{LUMO}−E¹_{HOMO})≤0.5 Equation 1
wherein, in Equation 1,
E¹_{LUMO} indicates a lowest unoccupied molecular orbital (LUMO) energy level of the first compound,
E¹_{HOMO} indicates a highest occupied molecular orbital (HOMO) energy level of the first compound, and
E²_{LUMO} indicates a LUMO energy level of the second compound.

2. The opto-electronic device of claim 1, wherein the opto-electronic device satisfies Equation 2:
0.25 eV≤E¹_{LUMO}−E²_{LUMO} Equation 2
wherein, in Equation 2,
E¹_{LUMO} and E²_{LUMO} are each as defined in claim 1.

3. The opto-electronic device of claim 1, wherein the opto-electronic device satisfies Equation 3:
E²_{HOMO}≤E¹_{HOMO} Equation 3
wherein, in Equation 3,
E¹_{HOMO} is as defined in claim 1, and
E²_{HOMO} indicates a HOMO energy level of the second compound.

4. The opto-electronic device of claim 1, wherein the opto-electronic device satisfies Equation 4:
3 eV≤E²_{LUMO}−E²_{HOMO}≤4 eV Equation 4
wherein, in Equation 4,
E²_{LUMO} is as defined in claim 1, and
E²_{HOMO} indicates a HOMO energy level of the second compound.

5. The opto-electronic device of claim 1, wherein a maximum absorption wavelength of the first compound is in a range of about 500 nm to about 600 nm.

6. The opto-electronic device of claim 1, wherein the first compound does not comprise fullerene.

7. The opto-electronic device of claim 1, wherein the first compound is selected from Compounds D1 to D4:

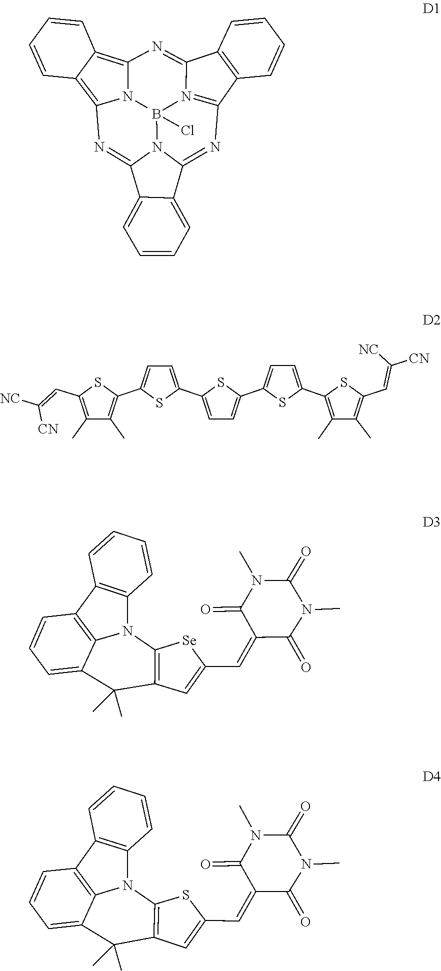

8. The opto-electronic device of claim 1, wherein the second compound is represented by Formula 1: 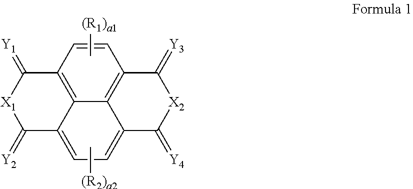
wherein, in Formula 1,
X₁ is O or N(Ar₁),
X₂ is O or N(Ar₂),
Ar₁ and Ar₂ are each independently hydrogen, deuterium, a C₁-C₆₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
Y₁ is O or C(R₁₁)(R₁₂),
Y₂ is O or C(R₂₁)(R₂₂),
Y₃ is O or C(R₃₁)(R₃₂),
Y₄ is O or C(R₄₁)(R₄₂),
a1 and a2 are each independently 0, 1, or 2,
R₁, R₂, R₁₁, R₁₂, R₂₁, R₂₂, R₃₁, R₃₂, R₄₁, and R₄₂ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

9. The opto-electronic device of claim 8, wherein, when X₁ and X₂ are each O, R₁ and R₂ are each not —F, —Cl, —Br, —I, or a cyano group.

10. The opto-electronic device of claim 8, wherein X₁ is N(Ar₁), and X₂ is N(Ar₂),
Ar₁ and Ar₂ are each independently a C₁-C₆₀ alkyl group unsubstituted or substituted with at least one R₁₀ₐ or a C₆-C₆₀ aryl group unsubstituted or substituted with at least one R₁₀ₐ, and
R₁₀ₐ is as defined in claim 8.

11. The opto-electronic device of claim 8, wherein Y₁ to Y₄ are each O.

12. The opto-electronic device of claim 1, wherein the second compound is one of Compounds A1 to A9:

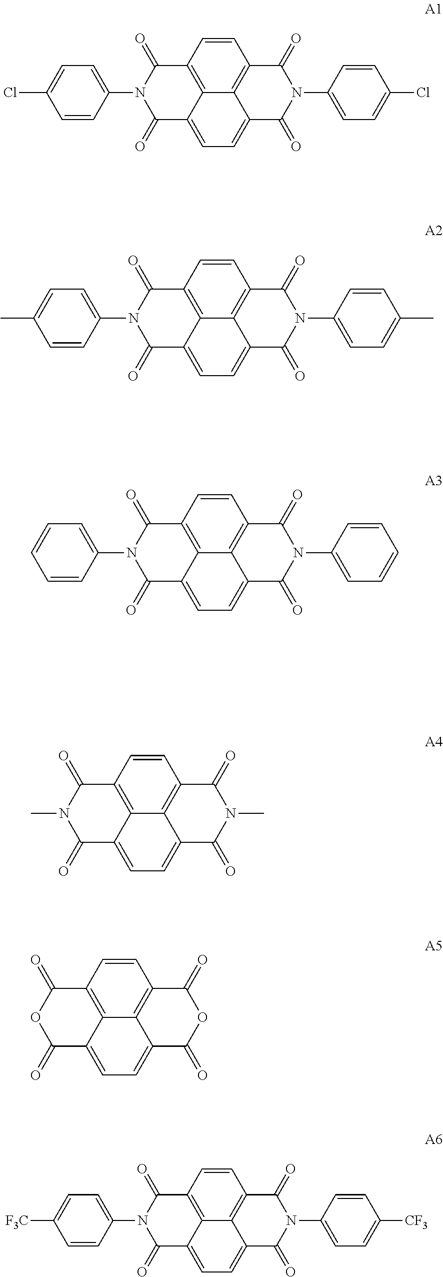

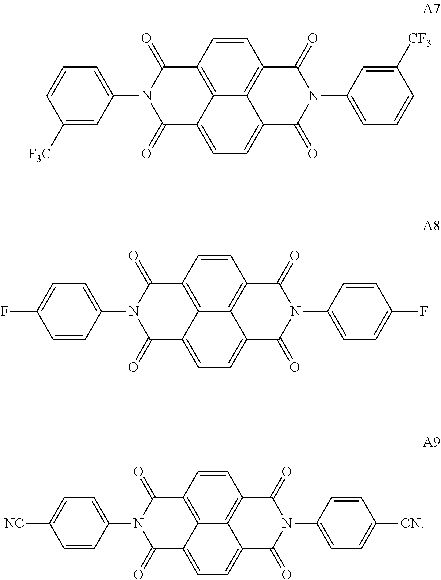 [SMILES: O=C1c2ccc3c4c(ccc(c24)C(=O)N1c1cccc(C(F)(F)F)c1)C(=O)N(c1cccc(C(F)(F)F)c1)C3=O ; O=C1c2ccc3c4c(ccc(c24)C(=O)N1c1ccc(F)cc1)C(=O)N(c1ccc(F)cc1)C3=O ; N#Cc1ccc(N2C(=O)c3ccc4c5c(ccc(c35)C2=O)C(=O)N(c2ccc(C#N)cc2)C4=O)cc1]

13. The opto-electronic device of claim 1, wherein the first layer is arranged between the first electrode and the second layer.

14. The opto-electronic device of claim 1, wherein the first layer is in contact with the second layer.

15. The opto-electronic device of claim 1, further comprising:
a hole transport region arranged between the first electrode and the photoactive layer; and
an electron transport region arranged between the photoactive layer and the second electrode,
wherein the hole transport region comprises a hole injection layer, a hole transport layer, an emission auxiliary layer, an electron-blocking layer, or any combination thereof, and
the electron transport region comprises a buffer layer, a hole-blocking layer, an electron control layer, an electron transport layer, an electron injection layer, or any combination thereof.

16. The opto-electronic device of claim 15, wherein the first layer is in contact with the hole transport region.

17. The opto-electronic device of claim 15, wherein the second layer is in contact with the electron transport region.

18. The opto-electronic device of claim 1, wherein the photoactive layer is formed by vacuum deposition.

19. An electronic apparatus comprising the opto-electronic device of claim 1.

20. The electronic apparatus of claim 19, further comprising:
a thin-film transistor electrically connected to the first electrode; and
a color filter, a color conversion layer, a touch screen layer, a polarizing layer, or any combination thereof.
